SEQUENCE LISTING (25 sequences)

SEQ ID NO: 1 - DNA, 1494 bp, Homo sapiens
atgagaacacaaattgaagtgatcccttgcaaaatctgtggggacaagtcgtctgggatccactacggggttatcacctgtgaggggtgcaagggcttcttccgccggagccagcgctgtaacgcggcctactcctgcacccgtcagcagaactgccccatcgaccgcaccagccgaaaccgatgccagcactgccgcctgcagaaatgcctggcgctgggcatgtcccgagatgctgtcaagttcggccgcatgtccaagaagcagagggacagcctgcatgcagaagtgcagaaacagctgcagcagcggcaacagcagcaacaggaaccagtggtcaagacccctccagcaggggcccaaggagcagataccctcacctacaccttggggctcccagacgggcagctgcccctgggctcctcgcctgacctgcctgaggcttctgcctgtccccctggcctcctgaaagcctcaggctctgggccctcatattccaacaacttggccaaggcagggctcaatggggcctcatgccaccttgaatacagccctgagcggggcaaggctgagggcagagagagcttctatagcacaggcagccagctgacccctgaccgatgtggacttcgttttgaggaacacaggcatcctgggcttggggaactgggacagggcccagacagctacggcagccccagtttccgcagcacaccggaggcaccctatgcctccctgacagagatagagcacctggtgcagagcgtctgcaagtcctacagggagacatgccagctgcggctggaggacctgctgcggcagcgctccaacatcttctcccgggaggaagtgactggctaccagaggaagtccatgtgggagatgtgggaacggtgtgcccaccacctcaccgaggccattcagtacgtggtggagttcgccaagaggctctcaggctttatggagctctgccagaatgaccagattgtgcttctcaaagcaggagcaatggaagtggtgctggttaggatgtgccgggcctacaatgctgacaaccgcacggtcttttttgaaggcaaatacggtggcatggagctgttccgagccttgggctgcagcgagctcatcagctccatctttgacttctcccactccctaagtgccttgcacttttccgaggatgagattgccctctacacagcccttgttctcatcaatgcccatcggccagggctccaagagaaaaggaaagtagaacagctgcagtacaatctggagctggcctttcatcatcatctctgcaagactcatcgccaaagcatcctggcaaagctgccacccaaggggaagcttcggagcctgtgtagccagcatgtggaaaggctgcagatcttccagcacctccaccccatcgtggtccaagccgctttccctccactctacaaggagctcttcagcactgaaaccgagtcacctgtggggctgtccaagtga

SEQ ID NO: 2 - protein, 497 aa, Homo sapiens
MRTQIEVIPCKICGDKSSGIHYGVITCEGCKGFFRRSQRCNAAYSCTRQQNCPIDRTSRNRCQHCRLQKCLALGMSRDAVKFGRMSKKQRDSLHAEVQKQLQQRQQQQQEPVVKTPPAGAQGADTLTYTLGLPDGQLPLGSSPDLPEASACPPGLLKASGSGPSYSNNLAKAGLNGASCHLEYSPERGKAEGRESFYSTGSQLTPDRCGLRFEEHRHPGLGELGQGPDSYGSPSFRSTPEAPYASLTEIEHLVQSVCKSYRETCQLRLEDLLRQRSNIFSREEVTGYQRKSMWEMWERCAHHLTEAIQYVVEFAKRLSGFMELCQNDQIVLLKAGAMEVVLVRMCRAYNADNRTVFFEGKYGGMELFRALGCSELISSIFDFSHSLSALHFSEDEIALYTALVLINAHRPGLQEKRKVEQLQYNLELAFHHHLCKTHRQSILAKLPPKGKLRSLCSQHVERLQIFQHLHPIVVQAAFPPLYKELFSTETESPVGLSK

SEQ ID NO: 3 - DNA, 1488 bp, Mus musculus
atgagaacacaaattgaagtgatcccttgcaagatctgtggggacaagtcatctgggatccactacggggttatcacctgtgaggggtgcaagggcttcttccgccgcagccagcagtgtaatgtggcctactcctgcacgcgtcagcagaactgccccattgaccgaaccagccgcaaccgatgccagcattgccgcctgcagaagtgcctggctctgggcatgtcccgagatgctgtcaagtttggccgaatgtccaagaagcagagggacagtctacatgcagaagtgcagaaacaactgcaacagcagcagcaacaggaacaagtggccaagactcctccagctgggagccgcggagcagacacacttacatacactttagggctctcagatgggcagctaccactgggcgcctcacctgacctacccgaggcctctgcttgtccccctggcctcctgagagcctcaggctctggcccaccatattccaataccttggccaaaacagaggtccagggggcctcctgccaccttgagtatagtccagaacgaggcaaagctgaaggcagagacagcatctatagcactgacggccaacttactcttggaagatgtggacttcgttttgaggaaaccaggcatcctgaacttggggaaccagaacagggtccagacagccactgcattcccagtttctgcagtgccccagaggtaccatatgcctctctgacagacatagagtacctggtacagaatgtctgcaagtccttccgagagacatgccagctgcgactggaggaccttctacggcagcgcaccaacctcttttcacgggaggaggtgaccagctaccagaggaagtcaatgtgggagatgtgggagcgctgtgcccaccacctcactgaggccattcagtatgtggtggagtttgccaagcggctttcaggcttcatggagctctgccagaatgaccagatcatactactgaaagcaggagcaatggaagtcgtcctagtcagaatgtgcagggcctacaatgccaacaaccacacagtcttttttgaaggcaaatacggtggtgtggagctgtttcgagccttgggctgcagcgagctcatcagctccatatttgacttttcccacttcctcagcgccctgtgtttttctgaggatgagattgccctctacacggccctggttctcatcaatgccaaccgtcctgggctccaagagaagaggagagtggaacatctgcaatacaatttggaactggctttccatcatcatctctgcaagactcatcgacaaggcctcctagccaagctgccacccaaaggaaaactccggagcctgtgcagccaacatgtggaaaagctgcagatcttccagcacctccaccccatcgtggtccaagccgccttccctccactctataaggaactcttcagcactgatgttgaatcccctgaggggctgtcaaagtga

SEQ ID NO: 4 - protein, 495 aa, Mus musculus
MRTQIEVIPCKICGDKSSGIHYGVITCEGCKGFFRRSQQCNVAYSCTRQQNCPIDRTSRNRCQHCRLQKCLALGMSRDAVKFGRMSKKQRDSLHAEVQKQLQQQQQQEQVAKTPPAGSRGADTLTYTLGLSDGQLPLGASPDLPEASACPPGLLRASGSGPPYSNTLAKTEVQGASCHLEYSPERGKAEGRDSIYSTDGQLTLGRCGLRFEETRHPELGEPEQGPDSHCIPSFCSAPEVPYASLTDIEYLVQNVCKSFRETCQLRLEDLLRQRTNLFSREEVTSYQRKSMWEMWERCAHHLTEAIQYVVEFAKRLSGFMELCQNDQIILLKAGAMEVVLVRMCRAYNANNHTVFFEGKYGGVELFRALGCSELISSIFDFSHFLSALCFSEDEIALYTALVLINANRPGLQEKRRVEHLQYNLELAFHHHLCKTHRQGLLAKLPPKGKLRSLCSQHVEKLQIFQHLHPIVVQAAFPPLYKELFSTDVESPEGLSK

SEQ ID NO: 5 - DNA, 1557 bp, Homo sapiens
atggacagggccccacagagacagcaccgagcctcacgggagctgctggctgcaaagaagacccacacctcacaaattgaagtgatcccttgcaaaatctgtggggacaagtcgtctgggatccactacggggttatcacctgtgaggggtgcaagggcttcttccgccggagccagcgctgtaacgcggcctactcctgcacccgtcagcagaactgccccatcgaccgcaccagccgaaaccgatgccagcactgccgcctgcagaaatgcctggcgctgggcatgtcccgagatgctgtcaagttcggccgcatgtccaagaagcagagggacagcctgcatgcagaagtgcagaaacagctgcagcagcggcaacagcagcaacaggaaccagtggtcaagacccctccagcaggggcccaaggagcagataccctcacctacaccttggggctcccagacgggcagctgcccctgggctcctcgcctgacctgcctgaggcttctgcctgtccccctggcctcctgaaagcctcaggctctgggccctcatattccaacaacttggccaaggcagggctcaatggggcctcatgccaccttgaatacagccctgagcggggcaaggctgagggcagagagagcttctatagcacaggcagccagctgacccctgaccgatgtggacttcgttttgaggaacacaggcatcctgggcttggggaactgggacagggcccagacagctacggcagccccagtttccgcagcacaccggaggcaccctatgcctccctgacagagatagagcacctggtgcagagcgtctgcaagtcctacagggagacatgccagctgcggctggaggacctgctgcggcagcgctccaacatcttctcccgggaggaagtgactggctaccagaggaagtccatgtgggagatgtgggaacggtgtgcccaccacctcaccgaggccattcagtacgtggtggagttcgccaagaggctctcaggctttatggagctctgccagaatgaccagattgtgcttctcaaagcaggagcaatggaagtggtgctggttaggatgtgccgggcctacaatgctgacaaccgcacggtcttttttgaaggcaaatacggtggcatggagctgttccgagccttgggctgcagcgagctcatcagctccatctttgacttctcccactccctaagtgccttgcacttttccgaggatgagattgccctctacacagcccttgttctcatcaatgcccatcggccagggctccaagagaaaaggaaagtagaacagctgcagtacaatctggagctggcctttcatcatcatctctgcaagactcatcgccaaagcatcctggcaaagctgccacccaaggggaagcttcggagcctgtgtagccagcatgtggaaaggctgcagatcttccagcacctccaccccatcgtggtccaagccgctttccctccactctacaaggagctcttcagcactgaaaccgagtcacctgtggggctgtccaagtga

SEQ ID NO: 6 - protein, 518 aa, Homo sapiens
MDRAPQRQHRASRELLAAKKTHTSQIEVIPCKICGDKSSGIHYGVITCEGCKGFFRRSQRCNAAYSCTRQQNCPIDRTSRNRCQHCRLQKCLALGMSRDAVKFGRMSKKQRDSLHAEVQKQLQQRQQQQQEPVVKTPPAGAQGADTLTYTLGLPDGQLPLGSSPDLPEASACPPGLLKASGSGPSYSNNLAKAGLNGASCHLEYSPERGKAEGRESFYSTGSQLTPDRCGLRFEEHRHPGLGELGQGPDSYGSPSFRSTPEAPYASLTEIEHLVQSVCKSYRETCQLRLEDLLRQRSNIFSREEVTGYQRKSMWEMWERCAHHLTEAIQYVVEFAKRLSGFMELCQNDQIVLLKAGAMEVVLVRMCRAYNADNRTVFFEGKYGGMELFRALGCSELISSIFDFSHSLSALHFSEDEIALYTALVLINAHRPGLQEKRKVEQLQYNLELAFHHHLCKTHRQSILAKLPPKGKLRSLCSQHVERLQIFQHLHPIVVQAAFPPLYKELFSTETESPVGLSK

SEQ ID NO: 7 - DNA, 1551 bp, Mus musculus
atggacagggccccacagagacaccaccggacatctcgggagctgctggctgcaaagaagacccacacctcacaaattgaagtgatcccttgcaagatctgtggggacaagtcatctgggatccactacggggttatcacctgtgaggggtgcaagggcttcttccgccgcagccagcagtgtaatgtggcctactcctgcacgcgtcagcagaactgccccattgaccgaaccagccgcaaccgatgccagcattgccgcctgcagaagtgcctggctctgggcatgtcccgagatgctgtcaagtttggccgaatgtccaagaagcagagggacagtctacatgcagaagtgcagaaacaactgcaacagcagcagcaacaggaacaagtggccaagactcctccagctgggagccgcggagcagacacacttacatacactttagggctctcagatgggcagctaccactgggcgcctcacctgacctacccgaggcctctgcttgtccccctggcctcctgagagcctcaggctctggcccaccatattccaataccttggccaaaacagaggtccagggggcctcctgccaccttgagtatagtccagaacgaggcaaagctgaaggcagagacagcatctatagcactgacggccaacttactcttggaagatgtggacttcgttttgaggaaaccaggcatcctgaacttggggaaccagaacagggtccagacagccactgcattcccagtttctgcagtgccccagaggtaccatatgcctctctgacagacatagagtacctggtacagaatgtctgcaagtccttccgagagacatgccagctgcgactggaggaccttctacggcagcgcaccaacctcttttcacgggaggaggtgaccagctaccagaggaagtcaatgtgggagatgtgggagcgctgtgcccaccacctcactgaggccattcagtatgtggtggagtttgccaagcggctttcaggcttcatggagctctgccagaatgaccagatcatactactgacagcaggagcaatggaagtcgtcctagtcagaatgtgcagggcctacaatgccaacaaccacacagtcttttttgaaggcaaatacggtggtgtggagctgtttcgagccttgggctgcagcgagctcatcagctccatatttgacttttcccacttcctcagcgccctgtgtttttctgaggatgagattgccctctacacggccctggttctcatcaatgccaaccgtcctgggctccaagagaagaggagagtggaacatctgcaatacaatttggaactggctttccatcatcatctctgcaagactcatcgacaaggcctcctagccaagctgccacccaaaggaaaactccggagcctgtgcagccaacatgtggaaaagctgcagatcttccagcacctccaccccatcgtggtccaagccgccttcccgccactctataaggaactcttcagcactgatgttgaatcccctgaggggctgtcaaagtga

SEQ ID NO: 8 - protein, 516 aa, Mus musculus
MDRAPQRHHRTSRELLAAKKTHTSQIEVIPCKICGDKSSGIHYGVITCEGCKGFFRRSQQCNVAYSCTRQQNCPIDRTSRNRCQHCRLQKCLALGMSRDAVKFGRMSKKQRDSLHAEVQKQLQQQQQQEQVAKTPPAGSRGADTLTYTLGLSDGQLPLGASPDLPEASACPPGLLRASGSGPPYSNTLAKTEVQGASCHLEYSPERGKAEGRDSIYSTDGQLTLGRCGLRFEETRHPELGEPEQGPDSHCIPSFCSAPEVPYASLTDIEYLVQNVCKSFRETCQLRLEDLLRQRTNLFSREEVTSYQRKSMWEMWERCAHHLTEAIQYVVEFAKRLSGFMELCQNDQIILLTAGAMEVVLVRMCRAYNANNHTVFFEGKYGGVELFRALGCSELISSIFDFSHFLSALCFSEDEIALYTALVLINANRPGLQEKRRVEHLQYNLELAFHHHLCKTHRQGLLAKLPPKGKLRSLCSQHVEKLQIFQHLHPIVVQAAFPPLYKELFSTDVESPEGLSK

SEQ ID NO: 9 - DNA, 21 bp, Artificial Sequence
  synthetic
ggagcagacacacttacatac

SEQ ID NO: 10 - DNA, 21 bp, Artificial Sequence
  synthetic
ggaactggctttccatcatca

SEQ ID NO: 11 - DNA, 1819 bp, Homo sapiens
cccctgggccctgctccctgccctcctgggcagccagggcagccaggacggcaccaagggagctgccccatggacagggccccacagagacagcaccgagcctcacgggagctgctggctgcaaagaagacccacacctcacaaattgaagtgatcccttgcaaaatctgtggggacaagtcgtctgggatccactacggggttatcacctgtgaggggtgcaagggcttcttccgccggagccagcgctgtaacgcggcctactcctgcacccgtcagcagaactgccccatcgaccgcaccagccgaaaccgatgccagcactgccgcctgcagaaatgcctggcgctggggatgtcccgagatgctgtcaagttcggccgcatgtccaagaagcagagggacagcctgcatgcagaagtgcagaaacagctgcagcagcggcaacagcagcaacaggaaccagtggtcaagacccctccagcaggggcccaaggagcagataccctcacctacaccttggggctcccagacgggcagctgcccctgggctcctcgcctgacctgcctgaggcttctgcctgtccccctggcctcctgaaagcctcaggctctgggccctcatattccaacaacttggccaaggcagggctcaatggggcctcatgccaccttgaatacagccctgagcggggcaaggctgagggcagagagagcttctatagcacaggcagccagctgacccctgaccgatgtggacttcgttttgaggaacacaggcatcctgggcttggggaactgggacagggcccagacagctacggcagccccagtttccgcagcacaccggaggcaccctatgcctccctgacagagatagagcacctggtgcagagcgtctgcaagtcctacagggagacatgccagctgcggctggaggacctgctgcggcagcgctccaacatcttctcccgggaggaagtgactggctaccagaggaagtccatgtgggagatgtgggaacggtgtgcccaccacctcaccgaggccattcagtacgtggtggagttcgccaagaggctctcaggctttatggagctctgccagaatgaccagattgtgcttctcaaagcaggagcaatggaagtggtgctggttaggatgtgccgggcctacaatgctgacaaccgcacggtcttttttgaaggcaaatacggtggcatggagctgttccgagccttgggctgcagcgagctcatcagctccatctttgacttctcccactccctaagtgccttgcacttttccgaggatgagattgccctctacacagcccttgttctcatcaatgcccatcggccagggctccaagagaaaaggaaagtagaacagctgcagtacaatctggagctggcctttcatcatcatctctgcaagactcatcgccaaagcatcctggcaaagctgccacccaaggggaagcttcggagcctgtgtagccagcatgtggaaaggctgcagatcttccagcacctccaccccatcgtggtccaagccgctttccctccactctacaaggagctcttcagcactgaaaccgagtcacctgtgggctgtccaagtgacctggaagagggactccttgcctctccctatggcctgctggccacctccctggaccccgttccaccctcacccttttcctttcccatgaaccctggagggtggtccccaccagctctttggaagtgagcagatgctgcggctggctttctgtcagcaggccggcctggcagtgggacaatcgccagagggtggg

SEQ ID NO: 12 - protein, 560 aa, Homo sapiens
MDRAPQRQHRASRELLAAKKTHTSQIEVIPCKICGDKSSGIHYGVITCEGCKGFFRRSQRCNAAYSCTRQQNCPIDRTSRNRCQHCRLQKCLALGMSRDAVKFGRMSKKQRDSLHAEVQKQLQQRQQQQQEPVVKTPPAGAQGADTLTYTLGLPDGQLPLGSSPDLPEASACPPGLLKASGSGPSYSNNLAKAGLNGASCHLEYSPERGKAEGRESFYSTGSQLTPDRCGLRFEEHRHPGLGELGQGPDSYGSPSFRSTPEAPYASLTEIEHLVQSVCKSYRETCQLRLEDLLRQRSNIFSREEVTGYQRKSMWEMWERCAHHLTEAIQYVVEFAKRLSGFMELCQNDQIVLLKAGAMEVVLVRMCRAYNADNRTVFFEGKYGGMELFRALGCSELISSIFDFSHSLSALHFSEDEIALYTALVLINAHRPGLQEKRKVEQLQYNLELAFHHHLCKTHRQSILAKLPPKGKLRSLCSQHVERLQIFQHLHPIVVQAAFPPLYKELFSTETESPVGCPSDLEEGLLASPYGLLATSLDPVPPSPFSFPMNPGGWSPPALWK

SEQ ID NO: 13 - DNA, 3192 bp, Mus musculus
aagaggcgtgttggagctgactgaagaagtggggggggcaccatacacctgcgtctgcagcccaggctcatggtagtgaaatccagaaaaaaacattatgggctagcttctctttctctcttccagcacaaattgaagtgatcccttgcaagatctgtggggacaagtcatctgggatccactacggggttatcacctgtgaggggtgcaaggtgagttgtacatatttgtctgcatacatgcacttggctgtttcagcggtctccccagggtcaggaacaggagggaggaggaggacctaatctcgatgtaggaatgtgatcacagggtccatcacaattatacagtggaggttcggggactttggtggatgtagaaattcttgagaccagtgcacatgaattggaggtccctgggaccacctcaaactccgagagggtgggataagcagtttctgtttcccagggcttcttccgccgcagccagcagtgtaatgtggcctactcctgcacgcgtcagcagaactgccccattgaccgaaccagccgcaaccgatgccagcattgccgcctgcagaagtgcctggctctgggcatgtcccgagatggtgagaatgtcggggcagcccccaagagtccctgtactctccagagacatcctggctcttgccagagttggcttgcaaaaacagcctgtcaggggcctctttctgttcttctacccacagccctttgtcctgtctttttgagggtcctcaaggctcttcagcctctactccaggctctgctccagaaaacctttaccagcatcatcttagtactcttgtcccttcttcctctaatgagttctcttcattcttctttctcctggcctagttcctatccagttcccacactgttacctcctgccttgtttagctccatctccctcctgagtaactatccttaggcataggattgtggcaaggaacaccgtctgggttggcttgagaatgacccagaaggtgggggatcgagtgtggtgttcctcgagccgtatccacctcttcacccacctcctcacccaccgtcttcaccacaggaggagccctgggtggagtggggggcatgaggtgaggaagacccagaaggagcctgtcagcacttttcagtgctcaaaataacaaagccaaagcgaaaggaaacatgcaggggtgcaaaggggcaggcggggcgaagggctgtgcccccacacctgggaggggttgggggagtgaaaaggcaggaaagagagagcagaagaggatgttcagaagcaagccaccagagcctgggttgggctgtggtgagtatctaggtcaccagggagcctgcaggcctgaccacagggaaacctgtgttcgcagcccttctcttcctccagccctcccagacaggcaagctgaccccaatacagcctgaggccccttactcaacccccacctcagccctagtctcaggagactcgactccccggcccaagcccattgcttgagttctgcactattagcatctagtctagacctgaaagcattttgctggggagatggaaatgctggtggaaccgatcctaaagagagcagagccaggcatgaaaggagcctggagcctaagctccccaaaagaaagctcatagacagagctggggaaatggagcatggagtcagattcatgtgcctgttgccctgtcctgtcttggtcttctcttacctcctgtactgattcctgaaccttcttcagctgtcaagtttggccgaatgtccaagaagcagagggacagtctacatgcagaagtgcagaaacaactgcaacagcagcagcaacaggaacaagtggccaagactcctccagctgggagccgcggagcagacacacttacatacactttagggctctcagatgggcagctaccactgggcgcctcacctgacctacccgaggcctctgcttgtccccctggcctcctgagagcctcaggctctggcccaccatattccaataccttggccaaaacagaggtccagggggcctcctgccaccttgagtatagtccagaacgaggcaaagctgaaggcagagacagcatctatagcactgacggccaacttactcttggaagatgtggacttcgttttgaggaaaccaggcatcctgaacttggggaaccagaacagggtccagacagccactgcattcccagtttctgcagtgccccagaggtaccatatgcctctctgacagacataggtgagcatctgggaagggtgggggcagtgaaaatgagacaagaacttcctccagcacggtgcccatgtaatcaagcattagtcctaaggaattagggatcctggacaaaaagccaagggaggaggcagagcaagggaggccaggcagagggcctctttatgaactttggggtgggaactggctgatatctagctgtgacttcatcttctggccccagagtacctggtacagaatgtctgcaagtccttccgagagacatgccagctgcgactggaggaccttctacggcagcgcaccaacctcttttcacgggaggaggtgaccagctaccagaggaaggtaagggcaagagacatgagggaagggagggcaccaccacacgcgggtgcggtgcgggcgcgcgcgcgcgggacacacacacacacactggtagagcccagatatggcgtctttcacaggaatgggggtagcaatagggtatcacaggctggctaacaggtgtgactgtctgtatccaggcagcaagatgactatttctgtagctcctgcttattgagatttcacctagttagcaacctacatcctcttccacccagaccttgccacactgcctttcatcctccatcagtaactgatgctaatcagtatctgagggtcatttactggacaccctttcctgtcaggcattgtaatagaagctttgaattgtgttcaacctgctgtttgtgaactacaagtgtctaaagatagtgataaatgtggcccaatgcaaaatcatgagaatgtttttggttggttgattggttggttggttggttggttggttggttgttggttggatgttgttggttatttggttgtggttggttgttggttgttggttggttggttggttggttggttggttggttggttgattggtgttggt

SEQ ID NO: 14 - protein, 516 aa, Mus musculus; identical to SEQ ID NO: 8

SEQ ID NO: 15 - DNA, 22 bp, Artificial Sequence
  primer
ctccagaaggccctcagactac

SEQ ID NO: 16 - DNA, 24 bp, Artificial Sequence
  primer
agctttccctccgcattgacacag

SEQ ID NO: 17 - DNA, 27 bp, Artificial Sequence
  primer
nctgggaagctcagtgccgccaccagn

SEQ ID NO: 18 - DNA, 24 bp, Artificial Sequence
  primer
gaggataacactgtgagagttgac

SEQ ID NO: 19 - DNA, 21 bp, Artificial Sequence
  primer
gagttcatggtgctgtcttcc

SEQ ID NO: 20 - DNA, 28 bp, Artificial Sequence
  primer
ngttccccatgggattacaacatcactn

SEQ ID NO: 21 - DNA, 18 bp, Artificial Sequence
  primer
ccgctgagagggcttcac

SEQ ID NO: 22 - DNA, 22 bp, Artificial Sequence
  primer
tgcaggagtaggccacattaca

SEQ ID NO: 23 - DNA, 27 bp, Artificial Sequence
  primer
nagggcttcttccgccgcagccagcan

SEQ ID NO: 24 - DNA, 20 bp, Artificial Sequence
  primer
tggtgaaggtcggtgtgaac

SEQ ID NO: 25 - DNA, 24 bp, Artificial Sequence
  primer
ccatgtagttgaggtcaatgaagg

(19) United States
(12) Patent Application Publication
(10) Pub. No.: US 2017/0159057 A1
(43) Pub. Date: Jun. 8, 2017

(54) COMPOSITIONS AND METHODS FOR MODULATION OF RORGAMMAT FUNCTIONS

(71) Applicants: Dan Littman, New York, NY (US); Gerard Eberl, Paris (FR); Liang Zhou, Chicago, IL (US); Ivaylo Ivanov, New York, NY (US)

(72) Inventors: Dan Littman, New York, NY (US); Gerard Eberl, Paris (FR); Liang Zhou, Chicago, IL (US); Ivaylo Ivanov, New York, NY (US)

(21) Appl. No.: 15/358,668

(22) Filed: Nov. 22, 2016

Related U.S. Application Data
(63) Continuation of application No. 14/589,336, filed on Jan. 5, 2015, now abandoned.
Application No. 14/589,336 is a continuation of application No. 11/524,501, filed on Sep. 20, 2006, now abandoned.
Application No. 11/524,501 is a continuation-in-part of application No. PCT/US2005/022649, filed on Jun. 24, 2005.
(60) Provisional application No. 60/584,824, filed on Jul. 1, 2004.

(51) Int. Cl. C12N 15/113

(52) U.S. Cl. CPC C12N 15/1138; C12N 2310/531; C12N 2310/14

(57) ABSTRACT

The present invention relates to expression of RORγt in cells and tissues and the effect of expression of this gene on proliferation of specific immune cells and in promotion of immune cell aggregates and in induction of IL17 producing cells. Furthermore, the invention relates to methods and agents that may decrease function of the gene product (the protein) or expression of this gene in individuals experiencing an inflammatory condition, an autoimmune disease or a food allergy, or any other condition whereby it is desirable to inhibit an immune response. In addition, methods and agents useful for enhancing the function of RORγt with agonists or expression of this gene are also considered for use whereby it is desirable to increase immunity to a pathogen or tumor cell, for example, for use in conjunction with a vaccine. Screening methods for identifying novel modulators (antagonists and agonists) of RORγt are also disclosed.

### CROSS REFERENCE TO RELATED APPLICATIONS

This application is a Continuation-in-Part of International Application PCT/US2005/022649, filed Jun. 24, 2005, which in turn, claims benefit of priority under 35 U.S.C. 119(e) to Provisional Application Ser. No. 60/584,824, filed Jul. 1, 2004. Applicants claim the benefit of 35 U.S.C. §120 as to said International Application and the benefit of 35 U.S.C. §119(e) as to said Provisional Application and all of said applications are incorporated by reference herein in their entireties.

### FIELD OF THE INVENTION

This invention relates to novel methods and compositions for modulation of immunity In particular, the invention provides for a means of either enhancing immunity to a preselected antigen for which immunity is desired, or for diminishing the inflammation associated with an inflammatory disease or condition.

### BACKGROUND OF THE INVENTION

The gut-associated lymphoid tissue (GALT) includes mesenteric lymph nodes (mLNs), Peyer's patches (PPs), the appendix and isolated lymphoid follicles (ILFs) (H. Hamada et al., J Immunol 168, 57 (2002).) It also includes lymphocytes residing in the intestinal lamina propria (LPLs) and within the single layer of intestinal epithelial cells (intraepithelial lymphocytes, IELs) (D. Guy-Grand, P. Vassalli, Curr Opin Immunol 14, 255 (2002); A. Hayday, E. Theodoridis, E. Ramsburg, J. Shires, Nat Immunol 2, 997 (2001)). T cells present in the mLNs and PPs share the characteristics of mainstream peripheral αβ T cells (bearing the αβ T cell antigen receptor, TCR), whereas LPLs and IELs are enriched in γδ Tcells, and most IELs uniquely express CD8αα homodimers. In the absence of a thymus, CD8αα⁺, αβ and γδ IELs develop, and can be derived from bone marrow and fetal liver or intestine grafts into lymphopenic mice (B. Rocha, P. Vassalli, D. Guy-Grand, J Exp Med 180, 681 (1994); L. Lefrancois, S. Olson, J Immunol 159, 538 (1997); H. Saito et al., Science 280, 275 (1998).). These observations support the existence of an extrathymic pathway for the generation of IELs, at least in athymic or lymphopenic mice (D. Guy-Grand et al., J Exp Med 197, 333 (2003)). Following this argument, CD3⁻ IELs expressing the pre-Tα chain and germline T cell receptor (TCR) transcripts have been proposed to represent precursors of CD8αα⁺ αβ and γδ IELs (T. Lin et al., Eur J Immunol 24, 1080 (1994); S. T. Page et al., Proc Natl Acad Sci USA 95, 9459 (1998)). However, athymic mice have a 2-5 fold decrease in γδ IELs and an even greater reduction in CD8αα⁺ αβ IELs, suggesting that most IELs are derived from thymocytes (D. Guy-Grand, P. Vassalli, Curr Opin Immunol 14, 255 (2002); T. Lin, G. Matsuzaki, H. Kenai, K. Nomoto, Eur J Immunol 24, 1785 (1994)). In addition, a number of TCR transgenic models show that intestinal αβ and γδ IELs are generated in a context of negative thymic selection, i.e. in the presence of self-Ag in the thymus, while mainstream T cells are deleted (B. Rocha, H. von Boehmer, D. Guy-Grand, Proc Natl Acad Sci USA 89, 5336 (1992); D. Cruz et al., J Exp Med 188, 255 (1998); D. Guy-Grand et al., Eur J Immunol 31, 2593 (2001); A. J. Leishman et al., Immunity 16, 355 (2002); T. Lin et al., J Clin Invest 104, 1297 (1999); C. N. Levelt et al., Proc Natl Acad Sci USA 96, 5628 (1999). However, transgenic TCRs are expressed abnormally early during thymocyte differentiation, and it thus remains unclear if IELs normally skirt thymocyte negative selection.

Recently, small clusters of hematopoietic cells have been detected between crypts in the small intestine and have been named cryptopatches (CPs) (Y. Kanamori et al., J Exp Med 184, 1449 (1996)). CPs are absent in newborns, and gradually become more abundant after weaning to reach maximal numbers (1500-1700) in the adult intestine. A majority of cells present in CPs are hematopoietic CD3⁻c-kit⁺IL-7Rα⁺ cells (CP cells) that express low levels of CD3ε and germline TCR transcripts, but no pre-Tα chain (K. Suzuki et al., Immunity 13, 691 (2000)) or RAG-2 (D. Guy-Grand et al., J Exp Med 197, 333 (2003).). CP cells have been reported to give rise to 4 and γδ IELs upon their transfer into lymphopenic mice, and it has been suggested that they are progenitors for T cells that develop extrathymically in the gut (H. Saito et al., Science 280, 275 (1998). H. Saito et al., Science 280, 275 (1998); K. Suzuki et al., Immunity 13, 691 (2000)), although this interpretation has remained somewhat controversial (D. Guy-Grand et al. J Exp Med. 197:333 (2003)).

The “retinoic acid-related orphan receptor” or “RORγt” (also referred to as “RORgt”) is a member of the large family of hormone nuclear receptors that include receptors for steroids, retinoids, thyroid hormones, and vitamin D3 (Mangelsdorf D J, et al.; (1995) Cell; 83:835-839.). Nuclear receptors are potent regulators of development, cell differentiation, and organ physiology, and members of the ROR subfamily, in particular, are required for an array of developmental and physiological processes. The murine Rorg gene encodes two isoforms, RORγ and RORγt, produced probably by initiation from two distinct promoters, although differential splicing from non-coding upstream exons cannot currently be excluded. The 24 N-terminal residues of RORγ, encoded by the first two exons, are replaced by three alternative residues encoded by a first exon specific to RORγt (He Y W, Deftos M L, Ojala E W, Bevan M J. (1998); Immunity 9:797-806; Villey I, de Chasseval R, de Villartay J P. (1999); Eur J Immunol 29:4072-4080; Medvedev A, Yan Z H, Hirose T, Giguere V, Jetten A M (1996); Gene 181:199-206; Hirose T, Smith R J, Jetten A M. (1994); Biochem Biophys Res Commun 205:1976-1983; Medvedev A, Chistokhina A, Hirose T, Jetten A M. (1997); Genomics 46:93-102.). Whereas RORγ mRNA is detected in many tissues including liver, lung, muscle, heart, and brain, RORγt mRNA has been detected only in immature double-positive (DP) CD4+CD8+ thymocytes and in a fetal population of CD3⁻CD4+CD45+ cells (He Y W, Deftos M L, Ojala E W, Bevan M J. (1998); Immunity 9:797-806; Villey I, de Chasseval R, de Villartay J P. (1999); Eur J Immunol 29:4072-4080; Medvedev A, Yan Z H, Hirose T, Giguere V, Jetten A M (1996); Gene 181:199-206; Hirose T, Smith R J, Jetten A M. (1994); Biochem Biophys Res Commun 205:1976-1983; Medvedev A, Chistokhina A, Hirose T, Jetten A M. (1997); Genomics 46:93-102; Eberl, G. et al.; (2004), Nature Immunol 5 (1): 1-8) , shown to be involved in the development of lymph nodes (LNs) and Peyer's patches (PPs) (Mebius R E, Rennert P, Weissman I L; (1997); Immunity 7:493-504; Adachi S, Yoshida H, Kataoka H, Nishikawa S. (1997); Int Immunol 9:507-514; Mebius R E, Streeter P R, Michie S, Butcher E C, Weissman I L. (1996); Proc Natl Acad Sci USA 93:11019-11024; Cupedo T, Kraal G, Mebius R E. (2002); Immunol Rev 189:41-50).

During fetal life, RORγt is exclusively expressed in lymphoid tissue inducer (LTi) cells and is required for the generation of these cells (G. Eberl et al., Nat Immunol 5, 64 (2004)). In the adult, RORγt is expressed in and regulates the survival of double positive (DP) CD4⁺CD8⁺ immature thymocytes (Z. Sun et al., Science 288,2369 (2000)).

Effective adaptive immune responses to pathogenic and commensal microorganisms require that lymphocytes be endowed with effector properties appropriate for each challenge. In response to microbe-induced cues from the innate immune system, CD4⁺ helper T lymphocytes thus differentiate in peripheral tissues to adopt a variety of fates (Seder, R. A., and Paul, W. E. (1994), Acquisition of lymphokine-producing phenotype by CD4+ T cells. Annu Rev Immunol 12, 635-673). Much focus has been placed on two T helper cell subsets, the Th1 cells, which produce interferon-y (IFNγ), and the Th2 cells, which produce IL-4, IL-5, and IL-13 (Abbas, A. K., Murphy, K. M., and Sher, A. (1996). Functional diversity of helper T lymphocytes. Nature 383, 787-793; Glimcher, L. H., and Murphy, K. M. (2000). Lineage commitment in the immune system: the T helper lymphocyte grows up. Genes Dev 14, 1693-1711). Th1 cells control infections with intracellular pathogens, including viruses and bacteria, but they have also been implicated as the effectors in a variety of autoimmune diseases (Christen, U., and von Herrath, M. G. (2004). Manipulating the type 1 vs type 2 balance in type 1 diabetes Immunol Res 30, 309-325; Gately, M. K., Renzetti, L. M., Magram, J., Stern, A. S., Adorini, L., Gubler, U., and Presky, D. H. (1998). The interleukin-12/interleukin-12-receptor system: role in normal and pathologic immune responses. Annu Rev Immunol 16, 495-521; Trembleau, S., Germann, T., Gately, M. K., and Adorini, L. (1995). The role of IL-12 in the induction of organ-specific autoimmune diseases. Immunol Today 16, 383-386). Th1 differentiation requires the cytokine IL-12 and the transcription factors, STAT4, STAT1 and T-bet (Szabo, S. J., Kim, S. T., Costa, G. L., Zhang, X., Fathman, C. G., and Glimcher, L. H. (2000). A novel transcription factor, T-bet, directs Th1 lineage commitment. Cell 100, 655-669). Th2 cells control infections with helminths and other extracellular microbes, and are, in part, the effectors that mediate the immunopathology of allergic responses and asthma. Th2 cell differentiation requires the cytokine IL-4 and the transcription factors STAT6 and GATA3 (Zheng, W., and Flavell, R. A. (1997). The transcription factor GATA-3 is necessary and sufficient for Th2 cytokine gene expression in CD4 T cells. Cell 89, 587-596).

It has recently been recognized that in many of the mouse autoimmune disease models that have been attributed to Th1 cells, disease severity is actually increased in the absence of these cells. For example, experimental autoimmune encephalomyelitis (EAE) and collagen induced arthritis (CIA) are exacerbated in the absence of IFNγ, IFNγ-R, STAT1, or IL-12 p35 (Iwakura, Y., and Ishigame, H. (2006). The IL-23/IL-17 axis in inflammation. J Clin Invest 116, 1218-1222; McKenzie, B. S., Kastelein, R. A., and Cua, D. J. (2006). Understanding the IL-23-IL-17 immune pathway. Trends Immunol 27, 17-23). However, mice lacking the IL-12 p40 subunit, which is shared with IL-23, are resistant to these autoimmune diseases (Cua, D. J., Sherlock, J., Chen, Y., Murphy, C. A., Joyce, B., Seymour, B., Lucian, L., To, W., Kwan, S., Churakova, T., et al. (2003). Interleukin-23 rather than interleukin-12 is the critical cytokine for autoimmune inflammation of the brain. Nature 421, 744-748; Murphy, C. A., Langrish, C. L., Chen, Y., Blumenschein, W., McClanahan, T., Kastelein, R. A., Sedgwick, J. D., and Cua, D. J. (2003). Divergent pro- and antiinflammatory roles for IL-23 and IL-12 in joint autoimmune inflammation. J Exp Med 198, 1951-1957). This paradox has now been resolved by the characterization of a third subset of T helper cells that secrete IL-17 (also known as IL-17A) and IL-17F and expand in response to IL-23 independently of T-bet or STAT1 (Aggarwal, S., Ghilardi, N., Xie, M. H., de Sauvage, F. J., and Gurney, A. L. (2003). Interleukin-23 promotes a distinct CD4 T cell activation state characterized by the production of interleukin-17. J Biol Chem 278, 1910-1914; Harrington, L. E., Hatton, R. D., Mangan, P. R., Turner, H., Murphy, T. L., Murphy, K. M., and Weaver, C. T. (2005). Interleukin 17-producing CD4+ effector T cells develop via a lineage distinct from the T helper type 1 and 2 lineages. Nat Immunol 6, 1123-1132; Iwakura, Y., and Ishigame, H. (2006). The IL-23/IL-17 axis in inflammation. J Clin Invest 116, 1218-1222; McKenzie, B. S., Kastelein, R. A., and Cua, D. J. (2006). Understanding the IL-23-IL-17 immune pathway. Trends Immunol 27, 17-23; Park, H., Li, Z., Yang, X. O., Chang, S. H., Nurieva, R., Wang, Y. H., Wang, Y., Hood, L., Zhu, Z., Tian, Q., and Dong, C. (2005). A distinct lineage of CD4 T cells regulates tissue inflammation by producing interleukin 17. Nat Immunol 6, 1133-1141). Mice lacking IL-17 are resistant to CIA and EAE (Nakae, S., Nambu, A., Sudo, K., and Iwakura, Y. (2003). Suppression of immune induction of collagen-induced arthritis in IL-17-deficient mice J Immunol 171, 6173-6177), transfer of cells that produce IL-17 results in severe disease (Langrish, C. L., Chen, Y., Blumenschein, W. M., Mattson, J., Basham, B., Sedgwick, J. D., McClanahan, T., Kastelein, R. A., and Cua, D. J. (2005). IL-23 drives a pathogenic T cell population that induces autoimmune inflammation. J Exp Med 201, 233-240) and treatment of mice with a neutralizing anti-IL-17 mAb suppresses CNS autoimmune inflammation (Langrish, C. L., Chen, Y., Blumenschein, W. M., Mattson, J., Basham, B., Sedgwick, J. D., McClanahan, T., Kastelein, R. A., and Cua, D. J. (2005). IL-23 drives a pathogenic T cell population that induces autoimmune inflammation. J Exp Med 201, 233-240). The differentiation of the IL-17- and IL-17F-producing cells, now termed Th17 cells, was initially shown to be dependent on the presence, during antigen stimulation, of IL-23 produced by the antigen presenting cells (Aggarwal, S., Ghilardi, N., Xie, M. H., de Sauvage, F. J., and Gurney, A. L. (2003). Interleukin-23 promotes a distinct CD4 T cell activation state characterized by the production of interleukin-17. J Biol Chem 278, 1910-1914; Harrington, L. E., Hatton, R. D., Mangan, P. R., Turner, H., Murphy, T. L., Murphy, K. M., and Weaver, C. T. (2005). Interleukin 17-producing CD4+ effector T cells develop via a lineage distinct from the T helper type 1 and 2 lineages. Nat Immunol 6, 1123-1132; Langrish, C. L., Chen, Y., Blumenschein, W. M., Mattson, J., Basham, B., Sedgwick, J. D., McClanahan, T., Kastelein, R. A., and Cua, D. J. (2005). IL-23 drives a pathogenic T cell population that induces autoimmune inflammation. J Exp Med 201, 233-240; Park, H., Li, Z., Yang, X. O., Chang, S. H., Nurieva, R., Wang, Y. H., Wang, Y., Hood, L., Zhu, Z., Tian, Q., and Dong, C. (2005). A distinct lineage of CD4 T cells regulates tissue inflammation by producing interleukin 17. Nat Immunol 6, 1133-1141). Consistent with this observation, mice deficient for the p19 subunit of IL-23 failed to produce Th17 cells and were resistant to EAE, CIA, and inflammatory bowel disease (IBD) (Cua, D. J., Sherlock, J., Chen, Y., Murphy, C. A., Joyce, B., Seymour, B., Lucian, L., To, W., Kwan, S., Churakova, T., et al. (2003). Interleukin-23 rather than interleukin-12 is the critical cytokine for autoimmune inflammation of the brain. Nature 421, 744-748; Langrish, C. L., Chen, Y., Blumenschein, W. M., Mattson, J., Basham, B., Sedgwick, J. D., McClanahan, T., Kastelein, R. A., and Cua, D. J. (2005). IL-23 drives a pathogenic T cell population that induces autoimmune inflammation. J Exp Med 201, 233-240; Yen, D., Cheung, J., Scheerens, H., Poulet, F., McClanahan, T., McKenzie, B., Kleinschek, M. A., Owyang, A., Mattson, J., Blumenschein, W., et al. (2006). IL-23 is essential for T cell-mediated colitis and promotes inflammation via IL-17 and IL-6. J Clin Invest 116, 1310-1316).

Although IL-23 has a key role in Th17-mediated inflammation in vivo, recent studies have demonstrated that in vitro polarization of naïve CD4⁺ T cells towards the Th17 lineage requires a combination of T cell antigen receptor (TCR) stimulation and the cytokines TGF-β and IL-6, but is independent of IL-23 (Bettelli, E., Carrier, Y., Gao, W., Korn, T., Strom, T. B., Oukka, M., Weiner, H. L., and Kuchroo, V. K. (2006). Reciprocal developmental pathways for the generation of pathogenic effector TH17 and regulatory T cells. Nature 441, 235-238; Veldhoen, M., Hocking, R. J., Atkins, C. J., Locksley, R. M., and Stockinger, B. (2006). TGFbeta in the context of an inflammatory cytokine milieu supports de novo differentiation of IL-17-producing T cells. Immunity 24, 179-189). Rather, IL-23 may be required for maintaining the Th17 phenotype or for promoting survival and/or proliferation of these cells. Overexpression of TGF-β in vivo also results in a marked increase of encephalitogenic Th17 cells following immunization with MOG antigen and complete Freund's adjuvant (Bettelli, E., Carrier, Y., Gao, W., Korn, T., Strom, T. B., Oukka, M., Weiner, H. L., and Kuchroo, V. K. (2006). Reciprocal developmental pathways for the generation of pathogenic effector TH17 and regulatory T cells. Nature 441, 235-238). TGF-β had been thought of primarily as an anti-inflammatory cytokine, in part because it induces in vitro differentiation of Foxp3-expressing regulatory T cells (Tregs) (Chen, W., Jin, W., Hardegen, N., Lei, K. J., Li, L., Marinos, N., McGrady, G., and Wahl, S. M. (2003). Conversion of peripheral CD4+CD25− naive T cells to CD4+CD25+ regulatory T cells by TGF-beta induction of transcription factor Foxp3. J Exp Med 198, 1875-1886; Fantini, M. C., Becker, C., Monteleone, G., Pallone, F., Galle, P. R., and Neurath, M. F. (2004). Cutting edge: TGF-beta induces a regulatory phenotype in CD4+CD25− T cells through Foxp3 induction and down-regulation of Smad7. J Immunol 172, 5149-5153). Thus TGF-β may induce either anti-inflammatory Tregs or pro-inflammatory Th17 cells, depending on the presence of IL-6.

The intestinal tract harbors a large fraction of the body's lymphatic tissue. Most T lymphocytes within the intestinal epithelium and lamina propria have an effector phenotype and are thought to be involved in maintaining a homeostatic balance between the luminal commensal microflora and the tissues of the intestine (Mowat, A. M. (2003). Anatomical basis of tolerance and immunity to intestinal antigens. Nat Rev Immunol 3, 331-341). Innate immune signaling pathways involving Myd88 are required for repair of intestinal epithelium, suggesting that increased exposure of cells within the lamina propria to commensal microorganisms results in signals that activate regenerative processes (Rakoff-Nahoum, S., Paglino, J., Eslami-Varzaneh, F., Edberg, S., and Medzhitov, R. (2004). Recognition of commensal microflora by toll-like receptors is required for intestinal homeostasis. Cell 118, 229-241). The cells involved in such processes and the mediators of homeostatic signals have yet to be characterized. Organized lymphoid structures in the lamina propria, including cryptopatches and isolated lymphoid follicles (ILFs), may receive signals from dendritic cells that surround them and can sample luminal content (Hamada, H., Hiroi, T., Nishiyama, Y., Takahashi, H., Masunaga, Y., Hachimura, S., Kaminogawa, S., Takahashi-Iwanaga, H., Iwanaga, T., Kiyono, H., et al. (2002). Identification of multiple isolated lymphoid follicles on the antimesenteric wall of the mouse small intestine. J Immunol 168, 57-64; Kanamori, Y., Ishimaru, K., Nanno, M., Maki, K., Ikuta, K., Nariuchi, H., and Ishikawa, H. (1996). Identification of novel lymphoid tissues in murine intestinal mucosa where clusters of c-kit+ IL-7R+ Thyl+ lympho-hemopoietic progenitors develop. J Exp Med 184, 1449-1459; Newberry, R. D., and Lorenz, R. G. (2005). Organizing a mucosal defense. Immunol Rev 206, 6-21; Niess, J. H., Brand, S., Gu, X., Landsman, L., Jung, S., McCormick, B. A., Vyas, J. M., Boes, M., Ploegh, H. L., Fox, J. G., et al. (2005). CX3CR1-mediated dendritic cell access to the intestinal lumen and bacterial clearance. Science 307, 254-258; Rescigno, M., Urbano, M., Valzasina, B., Francolini, M., Rotta, G., Bonasio, R., Granucci, F., Kraehenbuhl, J. P., and Ricciardi-Castagnoli, P. (2001). Dendritic cells express tight junction proteins and penetrate gut epithelial monolayers to sample bacteria. Nat Immunol 2, 361-367). Differentiation of these lymphoid tissues, as well as lymph nodes and Peyer's patches, is dependent on the orphan nuclear receptor RORγt. This transcription factor is required for the development of lymphoid tissue inducer (LTi) cells and LTi-like cells that guide development of the lymphoid tissues within the fetus and in the adult intestine, respectively (Eberl, G., and Littman, D. R. (2004). Thymic origin of intestinal alphabeta T cells revealed by fate mapping of RORgammat+ cells. Science 305, 248-251; Sun, Z., Unutmaz, D., Zou, Y. R., Sunshine, M. J., Pierani, A., Brenner-Morton, S., Mebius, R. E., and Littman, D. R. (2000). Requirement for RORgamma in thymocyte survival and lymphoid organ development. Science 288, 2369-2373).

It is toward novel methods and compositions for the modulation of intestinal immunity that the present invention is directed. In particular, through use of heterozygous mice in which a green fluorescent protein (GFP) reporter is under control of the Rorγt gene (Rorc(γt)^{+/gfp} mice), the inventors of the present application contemplate that the discovery of RORγt agonists and antagonists may be beneficial in the treatment of inflammatory bowel diseases, autoimmune diseases and disorders or alternatively as a means of enhancing mucosal immunity against pathogens and tumors in subjects in need of such treatment.

All publications, patent applications, patents and other reference material mentioned are incorporated by reference in their entirety. In addition, the materials, methods and examples are only illustrative and are not intended to be limiting. The citation of references herein shall not be construed as an admission that such is prior art to the present invention.

### SUMMARY OF THE INVENTION

The invention relates to the role of RORγt⁺ as a key regulator of immune homeostasis. The invention further relates to the role of RORγt⁺ in the organization of lymphoid tissue, and more particularly to the role of RORγt as a key regulator of immune homeostasis in mucosal tissue, such as, but not limited to, the intestines. RORγt may play a role in immune homeostasis in other mucosal tissues, such as the oral or nasal cavities, as well as others. The invention further relates to the role of RORγt in regulating immune homeostasis in tissue other than mucosal tissue, such as in nervous system tissue (central nervous system tissue, for example, the brain or the spinal cord, or any peripheral nervous system tissue), and respiratory tissue, such as lung tissue. The tissue in which RORγt⁺ may play a role in immune homeostasis may be any tissue in which an inflammatory process may occur, or at any body site containing foci of inflammation, or inflammatory cells. The invention also provides for the role of RORγt as a key transcription factor that orchestrates the differentiation of IL-17 producing T helper lymphocytes, also referred to as Th17 cells. Since these cells are the major contributors to inflammatory conditions and autoimmune disease, the present invention provides for the potential of developing therapeutic agents that target RORγt for treating such inflammatory conditions and autoimmune diseases. Accordingly, one aspect of the present invention provides methods for identifying antagonists of RORγt for the treatment of inflammatory conditions or autoimmune diseases. Another aspect of the invention provides methods for identifying agonists of RORγt for enhancement of an immune response to a pre-selected antigen against which immunity is desired. More particularly, such agonists of RORγt may be used to enhance immunity to tumor cells, or to microbial pathogens, including bacteria, viruses, fungi, or parasites. It is also envisioned that the agonists may be beneficial when used in conjunction with a vaccine candidate, to aid in development of specific immunity to the vaccine candidate.

The present invention demonstrates that in mice rendered deficient for RORγt through breeding the Rorc(γt)^{GFP} allele to homozygosity, intestinal lin⁻c-kit⁺IL-7Rα⁺ cells and CPs are absent, and no intestinal GFP⁺ cells are observed. In these animals, isolated lymphoid follicles (ILFs) also fail to develop, as shown by the absence of B cell clusters characteristic of these structures (Kanamori Y, Ishimaru K, Nanno M, Maki K, Ikuta K, Nariuchi H, Ishikawa H; (1996); J. Exp. Med. 184:1449-1459; Suzuki K, Oida T, Hamada H, Hitotsumatsu O, Watanabe M, Hibi T, Yamamoto H, Kubota E, Kaminogawa S, Ishikawa H; (2000); Immunity 13:691-702). Although intestinal γδ T cells and CD11c⁺ cells are present in normal numbers in the mutant mice, there are substantial and specific reduction in all subsets of intestinal αβ T cells, including CD4⁻8⁻ (DN), CD4⁺, CD8αβ⁺, and CD8αα⁺ cells, as well as a reduction in B cells and IgA in the lamina propria and in the feces.

In addition, evidence has been provided for the presence of a subpopulation of RORγt⁺ T cells in the lamina propria of Rorc(γt)^{+/gfp} mice. In particular, evidence is provided showing that most of these RORγt⁺ T cells in the small intestine of Rorc(γt)^{+/gfp} mice express IL-17, and that this population of IL-17 producing T cells is absent in mice lacking RORγt. T helper (Th) cells produce IL-17 in response to the cytokine IL-23. More particularly, IL-17 is made in response to TGF-β plus IL-6, by way of RORγt, and IL-23 can then enhance this process, since its receptor is turned on by IL-6 acting through RORγt to induce its expression. (Langrish, C.L. et al. (2004), Immunol Rev. 202:96-105; Langrish, C. L. et al. (2005), J. Exp. Med. 201:233-240; van Epps, H. (2005), J. Exp. Med. 201: 163; Honey, K. (2005), Nature, 5:94; Bettelli, E. et al. (2005), J. Exp. Med. 201:169-171). This Th cell subset, termed Th17, has been proposed to have pro-inflammatory functions. The results presented herein show that RORγt is required for the development of the potentially pro-inflammatory Th17 cells.

The present invention further provides for the finding that the RORγt gene is expressed exclusively in fetal lymphoid tissue inducer (LTi) cells, in immature thymocytes, in intestinal lin⁻ c-kit⁺IL-7Rα⁺ cells and also in Th17 cells in the intestine and in inflammatory foci at other sites in the body. The present invention also shows that RORγt is necessary for the development of all secondary lymphoid tissue, plus intestinal cryptopatches (CPs) and isolated lymphoid follicles (ILFs), as well as for the efficient generation of αβ T cells. In addition, the results suggest that intestinal RORγt⁺ cells are equivalent in the adult to fetal LTi cells, and are thus likely to induce the formation of mucosal lymphoid tissue, such as ILFs, in response to intestinal flora or to various inflammatory stimuli.

Accordingly, in its broadest aspect, this invention provides for methods of enhancing or depressing immune cell activity or function by administering a modulator of RORγt activity, that is, an agonist or an antagonist of RORγt, respectively. In the instance where it is desirous of inhibiting inflammatory cell activity and/or function, such as in an inflammatory or autoimmune disease or condition, it would be beneficial to administer a RORγt antagonist. In an instance where it is desirous to enhance immune cell activity and/or function, such as in an individual suffering from a hyperproliferative or cancerous disease or condition, or in a person being vaccinated against a specific pathogen, it would be desirous to administer an agonist of RORγt. The modulator of RORγt (the agonist or antagonist) may be selected from the group consisting of a small organic molecule (synthetic or naturally isolated or derived), a protein or peptide, a nucleic acid (RNA or DNA), a carbohydrate or an antibody. The nucleic acid may be single stranded or double stranded. The nucleic acid may be an antisense molecule or a small interfering nucleic acid molecule, such as a siRNA or a shRNA. The antibody may be a monoclonal antibody or a polyclonal antibody. The antibody may be a single chain antibody. The antibody may be a chimeric antibody. The antibody may be a human antibody or a humanized antibody.

Accordingly, a first aspect of the invention provides a method for inhibiting the formation of immune cell aggregates in the gut of a mammal, comprising administering an inhibitor or antagonist of RORγt. In a particular embodiment, the aggregates comprise isolated lymphoid follicles, including colonic patches in the gut of a mammal. The invention thus provides for the use of an antagonist or inhibitor of RORγt for inhibition of formation of immune cell aggregates in an animal, preferably but not limited to the gut of the animal.

In one particular embodiment, the cells that are inhibited are DP thymocytes, cryptopatch (CP) cells and Th17 cells. In another particular embodiment, the cells that are inhibited are IL-17 producing RORγt⁺ T cells. In another embodiment, the CP cells are required for the development of isolated lymphoid follicles (ILFs). In yet another embodiment, the method for inhibiting the formation of immune cell aggregates in the gut results in a lack of formation of lymphocyte aggregates in the lamina propria and in development of intraepithelial lymphocytes. In yet another embodiment, the method further results in a reduction in the number of αβT cells, or in IL-17 producing RORγt⁺ T cells. In yet another particular embodiment, the αβ T cells may be selected from the group consisting of CD4⁻8⁻ T cells, CD4+ T cells, CD8αβ+ T cells, CD8αα+ T cells and Th-IL17 cells. In another embodiment, the reduction in αβT cells or in IL-17 producing RORγt⁺ T cells occurs in the intestine, and also in tissues containing lymphoid cells, such as, but not limited to lung, liver, spleen or any other lymphoid tissue or organ that may be involved in an inflammatory disease or condition.

A second aspect of the invention provides a method of treating an inflammatory disease or an autoimmune disease, comprising administering a modulator of RORγt. In one preferred embodiment, the modulator is an inhibitor or antagonist of RORγt. In another particular embodiment, the modulator is a stimulator or agonist of RORγt. The invention also provides for the use of a modulator of RORγt, preferably an antagonist or inhibitor of RORγt for treating inflammatory and/or autoimmune diseases or conditions in a mammal, and/or any other infectious disease such as a viral disease, known to induce immunopathological damage to the host, preferably a human, although the modulator may be used to treat other domestic or non-domestic animals, including but not limited to dogs, cats, horses, cows, pigs and rodents

In one particular embodiment, the inflammatory or autoimmune disease is selected from the group consisting of arthritis, diabetes, multiple sclerosis, uveitis, rheumatoid arthritis, psoriasis, osteoporosis, asthma, bronchitis, allergic rhinitis, chronic obstructive pulmonary disease, atherosclerosis, H. pylori infections and ulcers resulting from such infection, and inflammatory bowel diseases. In another particular embodiment, the inflammatory bowel disease is selected from the group consisting of Crohn's disease, ulcerative colitis, sprue and food allergies. In another particular embodiment, the inflammatory disease or condition involves any organ or tissue containing cells in which the presence and/or expression of RORγt has been demonstrated. In another particular embodiment, other diseases known to produce immunopathological damage in the host, which may benefit from treatment with a modulator of RORγt, may be selected from the group consisting of Hepatitis C virus, Influenza, SARS, and respiratory syncytial virus.

A third aspect of the invention provides a method of treating an infection in a mammal comprising administering a modulator of RORγt. In one particular embodiment, the modulator is a stimulator or agonist of RORγt. In another particular embodiment, the modulator is an inhibitor or antagonist of RORγt. The invention also provides for the use of a modulator of RORγt for treating an infectious disease or condition in a mammal, preferably a human, although the modulator may be used to treat other domestic or non-domestic animals, including but not limited to dogs, cats, horses, cows, pigs and rodents. The modulator may be an antagonist or an agonist of RORγt.

In a particular embodiment, the administering results in promotion of T cell development from T cell progenitors and promotion of the formation of tertiary lymphoid organs. In another particular embodiment, the administering results in an increase in the number of αβT cells. In another particular embodiment, the administering results in an increase in the number of RORγt⁺ T cells that produce IL-17. In yet another embodiment, the αβT cells are selected from the group consisting of CD4⁻8⁻ T cells, CD4+ T cells, CD8αβ+ T cells and CD8αα+ T cells.

A fourth aspect of the invention provides a method of inducing anti-tumor immunity in a mammal comprising administering an agonist or stimulator of RORγt. In a particular embodiment, a method for the development of specific immunity against tumors of the gastrointestinal tract, such as, but not limited to, tumors of the stomach, bowel and intestine is envisioned. In another particular embodiment, a method for development of specific immunity against a tumor other than those that arise in the gastrointestinal tract is envisioned. For example, treatment of a tumor of the lung, liver, pancreas, breast, bone and any other solid tumor or blood borne tumor is contemplated. The treatment with an agonist or stimulator of RORγt may result in inhibition of tumor cell growth or proliferation, or may result in preventing the further spread (metastasis) of the tumor cells to other tissues or organs. The agonist or stimulator of RORγt may be administered alone or in conjunction with a tumor cell vaccine or in conjunction with other anti-tumor therapies known to those skilled in the art. The agonist may be administered at the same time, prior to, or after the other therapies.

The invention also provides for the use of a modulator of RORγt for treating a cancerous disease or condition, or for increasing anti-tumor immunity in an animal having a cancerous condition. In one embodiment, the animal is preferably a human, although the modulator may be used to treat other domestic or non-domestic animals, including but not limited to dogs, cats, horses, cows, pigs and rodents. The modulator may be an antagonist or an agonist of RORγt

In another particular embodiment, the development of agonists that can function as adjuvants to elicit local anti-tumor immunity is envisioned. In yet another particular embodiment, the present invention provides for a means to reduce inflammation in tumors, as well as to reduce the angiogenesis and growth of the tumor that may accompany the inflammation, since inflammation is now thought to be accompanied by angiogenesis and growth of tumors. In another particular embodiment, the administering results in promotion of T cell development from T cell progenitors and promotion of the formation of tertiary lymphoid organs. In another particular embodiment, the administering results in an increase in numbers of αβT cells. In another particular embodiment, the administering results in an increase in numbers of RORγt⁺ T cells that produce IL-17. In yet another embodiment, the αβT cells are selected from the group consisting of CD4⁻8⁻ T cells, CD4+ T cells, CD8αβ+ T cells and CD8αα+ T cells.

A fifth aspect of the invention provides a method of increasing the number of T cells reactive to a specific antigen, comprising administering an agonist of RORγt in conjunction with, prior to, or subsequent to the administration of the antigen. The agonist may be mixed with the vaccine prior to delivery and administered as a combination, or the agonist may be administered to a different site or by a different route from the site of injection of the vaccine or the route of administration of the vaccine.

A sixth aspect of the invention provides a method of increasing the immunogenicity of a vaccine candidate, wherein an increase in T cell proliferation and responsiveness by said vaccine candidate is desirable, comprising administering to a subject in conjunction with, prior to, or subsequent to said vaccine candidate, an immunogenicity promoting amount of an agonist to RORγt.

In a particular embodiment, the vaccine candidate is an attenuated live vaccine or a non-replicating and/or subunit vaccine, and the method results in induction of cytolytic or memory T cells specific for the vaccine candidate. In yet another embodiment, the vaccine is selected from the group consisting of a tumor vaccine, a viral vaccine, a bacterial vaccine, a parasitic vaccine and vaccines for other pathogenic organisms for which a long lasting immune response is necessary to provide long term protection from infection or disease. In yet another embodiment, the viral vaccine is selected from the group consisting of a DNA viral vaccine, an RNA viral vaccine and a retroviral viral vaccine. In another aspect, the vaccine is a “naked DNA vaccine” whereby genetic material (e.g., nucleic acid sequences) is used as the immunizing agent. Thus, the present invention relates to the introduction of exogenous or foreign DNA molecules into an individual's tissues or cells, wherein these molecules encode an exogenous protein capable of eliciting an immune response to the protein. The exogenous nucleic acid sequences may be introduced alone or in the context of an expression vector wherein the sequences are operably linked to promoters and/or enhancers capable of regulating the expression of the encoded proteins.

A seventh aspect of the invention provides a method of increasing mucosal immunity to a preselected antigen, comprising administering to a subject in conjunction with or subsequent to said antigen, a mucosal immunity promoting amount of an agonist to RORγt.

In a particular embodiment, the antigen is selected from the group consisting of a bacteria, a virus, a tumor cell and any other pathogen for which increased mucosal immunity is desired.

An eighth aspect of the invention provides a method of treating cancers of T cell origin, comprising administering an antagonist of RORγt.

In a particular embodiment, the cancers may be selected from the group consisting of acute T lymphatic leukemia (T-ALL), chronic T lymphatic leukemia (T-CLL), adult T cell leukemia (ATL), non-ATL peripheral T lymphoma (PNTL), Hodgkin's, non-Hodgkin's lymphoma and other leukemias and lymphomas exhibiting a double-positive, CD4+, CD8+ phenotype.

A ninth aspect of the invention provides for a method of measuring or detecting the level of RORγt in a tissue sample from a subject, whereby the presence of RORγt in a tissue sample is indicative of the presence of, or the potential for developing, an inflammatory or autoimmune disease or other diseases or conditions characterized by an increase in inflammatory cell numbers or activity. Such conditions may include an inflammatory bowel disease, rheumatoid arthritis, type I diabetes or a food allergy. Alternatively, the absence of RORγt may be indicative of an inability to mount a proper immune response to a pathogenic organism or tumor in a subject showing the absence of RORγt. Accordingly, the ability to measure the presence or absence of RORγt in an individual may aid in the ability to determine the appropriate treatment strategy for such condition. The method of measuring the level of RORγt in a subject comprises contacting a biological sample with a ligand and detecting said ligand bound to RORγt in the sample, wherein the detection of ligand bound to RORγt is indicative of an inflammatory condition or an autoimmune disease. In a particular embodiment, the ligand is an antibody, or a derivative or fragment thereof, which specifically binds to RORγt in the sample.

In another embodiment, the ability to measure RORγt in a sample may be accomplished using a nucleotide probe specific for RORγt. Techniques well known in the art, e.g., quantitative or semi-quantitative RT PCR, real-time PCR, or Northern blot, or gene chip analysis (microarrays) can be used to measure expression levels of RORγt. In another particular embodiment, the tissue sample is a biopsy sample. Any of these procedures may be utilized to assess the level of RORγt in a sample as a means of estimating the amount of inflammation present in the tissue of a patient, or to provide a means of assessing whether a therapeutic strategy has been effective in diminishing the inflammation in a patient suffering from an inflammatory disease or disorder or from an injury.

In a yet further embodiment, the method for determining in a biological sample the concentration of RORγt, comprises:
a. contacting said sample with a ligand under conditions wherein said ligand can form a complex with RORγt contained in the sample; and
b. determining the amount of RORγt and of RORγt bound by said ligand by detecting the amount of complex formed, wherein said detecting is accomplished by use of a radiolabel, an enzyme, a chromophore or a fluorescent probe.

In yet another particular embodiment, the method provides for screening, diagnosis or prognosis of a disease in a subject, wherein the disease is characterized by high levels of RORγt, wherein the disease is selected from the group consisting of arthritis, diabetes, multiple sclerosis, uveitis, rheumatoid arthritis, psoriasis, asthma, bronchitis, allergic rhinitis, chronic obstructive pulmonary disease, atherosclerosis, H pylori infections and ulcers resulting from such infection, an inflammatory bowel disease, an autoimmune disease, and a food allergy. In yet another particular embodiment, the disease may be selected from an infectious disease, such as a viral disease, which results in immunopathology. These may be selected from the group consisting of hepatitis C, SARS, influenza and respiratory syncitial virus. The method comprises:
(I) measuring an amount of a RORγt gene or gene product in a tissue sample derived from the subject, wherein said RORγt gene or gene product is:
   (a) a DNA corresponding to SEQ ID NO: 1, or a nucleic acid derived therefrom;
   (b) a protein comprising SEQ ID NO: 2;
   (c) a nucleic acid comprising a sequence hybridizable to SEQ ID NO: 1, or its complement under conditions of high stringency, or a protein comprising a sequence encoded by said hybridizable sequence;
   (d) a nucleic acid at least 90% homologous to SEQ ID NO: 1, or its complement as determined using the NBLAST algorithm; or a protein encoded thereby; and
(II) comparing the amount of said RORγt gene product in said subject with the amount of RORγt gene product present in a normal tissue sample obtained from a subject who does not have a disease characterized by high levels of RORγt or in a predetermined standard, wherein an increase in the amount of said RORγt gene product in said subject compared to the amount in the normal tissue sample or pre-determined standard indicates the presence of an inflammatory or autoimmune disease in said subject.

In yet another embodiment, the method provides a diagnostic method for determining the predisposition, the onset or the presence of an inflammatory or autoimmune disease or a food allergy in a subject. The method comprises detecting in the subject the existence of a change in the level of RORγt gene or gene product, as set forth in SEQ ID NO: 1 and SEQ ID NO: 2, or detecting a polymorphism in the RORγt gene that affects the function of the protein. The method further comprises:
a) obtaining a tissue biopsy from said subject;
b) permeabilizing the cells in said tissue biopsy;
c) incubating said tissue biopsy or cells isolated from said tissue biopsy with one of the following:
   i) an antibody specific for the RORγt gene product, or an antibody specific for the gene product of an RORγt gene having a polymorphism that affects the function of the protein; or
   ii) a nucleic acid probe specific for the RORγt gene or a nucleic acid probe that hybridizes with an RORγt gene having a polymorphism that affects the function of the protein;
d) detecting and quantitating the amount of antibody or nucleic acid probe bound;
e) comparing the amount of antibody or nucleic acid probe bound in the biopsy sample in said subject to the amount of antibody or nucleic acid probe bound in a normal tissue or cellular sample; and
wherein the amount of labeled antibody or nucleic acid probe bound correlates directly with the predisposition, the onset or the presence of an inflammatory or autoimmune disease or a food allergy in said subject.

A tenth aspect of the invention provides a method of regulating or inducing Th 17 cell differentiation and/or transcription of IL-17 and IL-17F, comprising administering an effective amount of a RORγt agonist to a T cell. In one embodiment, the agonist is selected from the group consisting of a small organic molecule, a protein or peptide, a nucleic acid, and a carbohydrate.

In one embodiment, the method further comprises treating a T cell with a differentiation effective amount of IL-6, TGF-β and/or an agent effective for ligating an antigen receptor on the T cell, or an analog, derivative, mimic or active fragment thereof, or a combination of any of the foregoing, in combination with a RORγt agonist.

In another particular embodiment, the agent effective for ligating an antigen receptor on the T cell is selected from the group consisting of an anti-CD3 antibody or an anti-CD 28 antibody.

In another particular embodiment, the T cell is a CD4⁺ Tcell, or a CD8+ T cell, or a CD4⁺CD25⁻CD62⁺CD44⁻T cell.

An eleventh aspect of the invention provides a method of regulating or inducing Th 17 cell differentiation in a mammal, comprising administering an effective amount of an agonist of RORγt to the mammal. In one embodiment, the agonist is selected from the group consisting of a small organic molecule, a protein or peptide, a nucleic acid, and a carbohydrate.

In one particular embodiment, the method further comprises administering IL-6, and/or TGF-β and/or an agent effective for ligating an antigen receptor on a T cell, or an analog, derivative, mimic or active fragment thereof, or a combination of any of the foregoing.

In another particular embodiment, the administering results in the differentiation or induction of a Th 17 cell and/or the transcription of IL-17 and IL-17F in a T cell found in the intestines of the mammal.

In another particular embodiment, the Th 17 cell is found in the lamina propria.

A twelfth aspect of the invention provides a method of inhibiting the induction, expression and/or release of a pro-inflammatory cytokine, or a proinflammatory cytokine receptor, and/or a pro-inflammatory chemokine, or a proinflammatory chemokine receptor, comprising administering an inhibitor or antagonist of RORγt.

In one particular embodiment, the antagonist may be a small organic molecule, a protein or peptide, a nucleic acid, for example, either a DNA or an RNA molecule, including an antisense molecule or a small interfering nucleic acid molecule (eg. a siRNA molecule), a lipid, a carbohydrate, an antibody or a fragment thereof. The antibody may be a monoclonal antibody or a polyclonal antibody, a chimeric antibody, a human or humanized antibody or a single chain antibody.

In one particular embodiment, the pro-inflammatory cytokine is selected from the group consisting of IL-17, IL-17F, IL-6, IL-21, IL-22, TNFsf8 and TNF-alpha.

In another particular embodiment, the proinflammatory cytokine receptor is selected from the group consisting of IL-23R, IL-1RI, IL-1RII, Cys1tr1, Ltb4r1 and IL-7Re.

In another particular embodiment, the pro-inflammatory chemokine is selected from the group consisting of CCL6, CCL9, CCL11, CCL20, CCL22, CCL24, and GM1960.

In another particular embodiment, the proinflammatory chemokine receptor is selected from the group consisting of CCR1, CCR2, CCR6, CCR9, CXCR7 and GPR43.

In yet another particular embodiment, the administering may be in vitro or in vivo.

In another particular embodiment, the in vivo administering results in a reduction in the severity of an inflammatory or autoimmune disease or condition, or an amelioration of one or more symptoms or sequelae associated with an inflammatory or autoimmune disease or condition.

In another particular embodiment, the inflammatory or autoimmune disease or condition is selected from the group consisting of arthritis, diabetes, multiple sclerosis, uveitis, rheumatoid arthritis, psoriasis, asthma, bronchitis, allergic rhinitis, chronic obstructive pulmonary disease, arteriosclerosis, H. pylori infections and ulcers resulting from such infection and an inflammatory bowel disease. In another particular embodiment, the inflammation may be in the brain or spinal cord as a result of a demyelinating disease or an injury.

In another particular embodiment, the inflammatory bowel disease is selected from the group consisting of Crohn's disease, ulcerative colitis, sprue and a food allergy.

A thirteenth aspect of the invention provides a method of enhancing the induction, expression and/or release of a pro-inflammatory cytokine, or a proinflammatory cytokine receptor, and/or a proinflammatory chemokine, or a proinflammatory chemokine receptor, comprising administering an agonist of RORγt.

In one particular embodiment, the pro-inflammatory cytokine is selected from the group consisting of IL-17, IL-17F, IL-6, IL-21, IL-22, TNFsf8 and TNF-alpha.

In one particular embodiment, the pro-inflammatory cytokine receptor is selected from the group consisting of IL-23R, IL-1RI, IL-1RII, Cys1tr1, Ltb4r1 and IL-7Re.

In another particular embodiment, the pro-inflammatory chemokine is selected from the group consisting of CCR1, CCR2, CCR6, CCR9, CXCR7 and GPR43.

In another particular embodiment, the pro-inflammatory chemokine receptor is selected from the group consisting of CCR6 and CCR9.

In another particular embodiment, the administering is in vitro or in vivo.

In another particular embodiment, the in vivo administering results in an enhancement of an immune response to a pre-selected antigen when the pre-selected antigen is administered in conjunction with, prior to, or shortly after the administering of the RORγt agonist.

In another particular embodiment, the antigen is isolated from a tumor cell or pathogen selected from the group consisting of a bacterium, a virus, a fungus and a parasite.

A fourteenth aspect of the invention provides a pharmaceutical composition comprising a RORγt receptor modulator, and a pharmaceutically acceptable carrier.

In one embodiment, the composition comprises a RORγt antagonist for inhibiting the induction, expression or release of one or more pro-inflammatory cytokines or chemokines from a cell, such that administering such composition to a subject suffering from an inflammatory condition or autoimmune disease may benefit from such treatment by having one or more symptoms or sequelae of the disease or condition ameliorated.

In another embodiment, the composition comprises a RORγt agonist for enhancing an immune response to a pre-selected antigen, and a pharmaceutically acceptable carrier. The administering of such a composition to a subject results in the induction, expression or release of one or more pro-inflammatory cytokines or chemokines from a cell. The enhanced induction, expression or release of the pro-inflammatory cytokines or chemokines then results in an increase in an immune response to the pre-selected antigen.

In another particular embodiment, the pharmaceutical composition comprises the RORγt antagonist alone or in combination with one or more compounds or agents effective for treating an inflammatory condition or an autoimmune disease. Alternatively, the pharmaceutical composition comprises the RORγt agonist alone or in combination with one or more compounds or agents effective for enhancing an immune response to a pre-selected antigen. The RORγt antagonist or agonist and the one or more compounds or agents may be formulated and administered alone or together. The pharmaceutical composition(s) comprising the RORγt antagonist or agonist and the one or more compounds or agents may be administered concurrently or sequentially. The pharmaceutical compositions may be delivered orally or parenterally. They may be delivered via the intravenous route, the intramuscular route, or the subcutaneous route. They may be delivered as an immediate release formulation or as a slow or sustained release formulation.

A fifteenth aspect of the invention provides a method of screening for a candidate compound that blocks or inhibits RORγt expression or activity/function, wherein the blocking or inhibiting results in a reduction in the expression or activity of one or more molecules associated with inflammation, wherein the molecules are selected from the group consisting of proinflammatory cytokines, proinflammatory cytokine receptors, proinflammatory chemokines, and proinflammatory chemokine receptors. In one embodiment, the method comprises:
(a) contacting the RORγt molecule, or fragments thereof, or cells containing the RORγt molecule, with a candidate compound in the presence or absence of a known inhibitor, wherein said RORγt molecule has a nucleic acid sequence of any one of SEQ ID NOs: 1 or 3 and/or the amino acid sequence of any one of SEQ ID NOs: 2 or 4; and
(b) determining the level of RORγt expression or activity/function in the presence or absence of the candidate compound;

wherein the candidate compound is considered to be effective if the level of RORγt expression or activity/function is lower in the presence of the candidate compound as compared to in the absence of the candidate compound.

In another particular embodiment, the method may comprise a following additional step of:
c) determining the level of expression or activity/function of one or more molecules associated with inflammation, wherein the molecules are selected from the group consisting of proinflammatory cytokines, proinflammatory cytokine receptors, proinflammatory chemokines, and proinflammatory chemokine receptors, and wherein a candidate compound is identified as a positive candidate compound if a decrease in the level of expression or activity/function of one or more proinflammatory cytokines, proinflammatory cytokine receptors, proinflammatory chemokines, or proinflammatory chemokine receptors is observed in the presence of the candidate compound, but not in the absence of the candidate compound.

In yet another particular embodiment, the pro-inflammatory cytokines are selected from the group consisting of IL-17, IL-17F, IL-6, IL-21, IL-22, TNFsf8 and TNF-alpha. In yet another particular embodiment, the pro-inflammatory cytokine receptors are selected from the group consisting of IL-23R, IL-1RI, IL-1RII, Cys1tr1, Ltb4r1 and IL-7Re. In yet another particular embodiment, the pro-inflammatory chemokines are selected from the group consisting of CCL6, CCL9, CCL11, CCL22, CCL24 and GM1960. In yet another particular embodiment, the pro-inflammatory chemokine receptors are selected from the group consisting CCR1, CCR2, CCR6, CCR9, CXCR7 and GPR43.

In yet another particular embodiment, the determining of the expression or activity/function is achieved by a method selected from the group consisting of reverse transcription-polymerase chain reaction (RT-PCR), real time PCR, northern blot analysis, in situ hybridization, cDNA microarray, electrophoretic gel analysis, an enzyme immunoassay (ELISA assays), a Western blot, a dotblot analysis, a protein microarray, a flow cytometric technique and proteomics analysis. Any one or more of these procedures may be used to determine or measure the expression or activity/function of RORγt, or of a proinflammatory cytokine or cytokine receptor, or of a proinflammatory chemokine or chemokine receptor.

A sixteenth aspect of the invention provides a method of screening for a candidate compound capable of modulating the expression or activity/function of RORγt. The modulating may refer to either enhancing or increasing the expression and/or activity of RORγt (an agonist) or decreasing the expressing and/or activity of RORγt (an antagonist). In one particular embodiment, the method comprises:
(a) contacting the RORγt molecule, or a cell containing RORγt, with a candidate compound, wherein the RORγt molecule is:
   (i) a DNA corresponding to either one of SEQ ID NOs: 1 or 3;
   (ii) a protein comprising either one of SEQ ID NOs: 2 or 4;
   (iii) a nucleic acid comprising a sequence hybridizable to either one of SEQ ID NOs: 1 or 3, or a complement thereof under conditions of high stringency, or a protein comprising a sequence encoded by said hybridizable sequence; or
   (iv) a nucleic acid at least 90% homologous to either one of SEQ ID NOs: 1 or 3, or a complement thereof as determined using an NBLAST algorithm or a protein encoded thereby;
(b) determining whether or not the candidate compound modulates the expression or activity/function of the RORγt molecule;
wherein a candidate compound that increases the expression or activity/function of the RORγt molecule is considered to be an agonist of RORγt, and wherein a candidate compound that decreases the expression or activity/function of the RORγt molecule is considered to be an antagonist of RORγt.

In another particular embodiment, the method may comprise the following step of:
c) determining the level of expression or activity/function of one or more molecules associated with inflammation, wherein said molecules are selected from the group consisting of proinflammatory cytokines, proinflammatory cytokine receptors, proinflammatory chemokines, or proinflammatory chemokine receptors,

wherein a candidate compound is identified as an agonist of RORγt if the candidate compound increases the expression or activity/function of one or more proinflammatory cytokines, proinflammatory cytokine receptors, proinflammatory chemokines, or proinflammatory chemokine receptors; and

wherein a candidate compound is identified as an antagonist of RORγt if the candidate compound decreases the expression or activity/function of one or more proinflammatory cytokines, proinflammatory cytokine receptors, proinflammatory chemokines, or proinflammatory chemokine receptors.

In another particular embodiment, the pro-inflammatory cytokines are selected from the group consisting of IL-17, IL-17F, IL-6, IL-21, IL-22, TNFsf8 and TNF-alpha. In yet another particular embodiment, the pro-inflammatory cytokine receptors are selected from the group consisting of IL-23R, IL-1RI, IL-1RII, Cys1tr1, Ltb4r1 and IL-7Re. In yet another particular embodiment, the pro-inflammatory chemokines are selected from the group consisting of CCL6, CCL9, CCL11, CCL22, CCL24 and GM1960. In yet another particular embodiment, the pro-inflammatory chemokine receptors are selected from the group consisting CCR1, CCR2, CCR6, CCR9, CXCR7 and GPR43.

In yet another particular embodiment, the determining of expression or activity/function is achieved by a method selected from the group consisting of reverse transcription-polymerase chain reaction (RT-PCR), real time PCR, northern blot analysis, in situ hybridization, cDNA microarray, electrophoretic gel analysis, an enzyme immunoassay (ELISA assays), a Western blot, a dotblot analysis, a protein microarray, a flow cytometric technique and proteomics analysis.

A seventeenth aspect of the invention provides a method of modulating interferon gamma (IFNγ) expression or production comprising treating a cell or an animal with a modulator of RORγt expression or activity/function. In one embodiment, treating a cell or an animal with a RORγt agonist would result in a downregulation or decrease in the expression or production of IFNγ. In another embodiment, treating a cell or an animal with a RORγt antagonist would result in an upregulation or increase in the expression or production of IFNγ.

### BRIEF DESCRIPTION OF THE DRAWINGS

FIG. 1. RORγt expression in the adult mouse. (A) RORγt⁺ cells in intestinal lymphoid tissues. Longitudinal sections of small intestine and colon of adult Rorc(γt)^{+/GFP} mice were stained as indicated, as well as for GFP (green). Cryptopatches (CP), small follicles (ILFs) and Peyer's patches (PP) are from the small intestine, and large follicles (ILFs) are from the colon. The relative size of these different structures is compared in the first row. Magnifications are 400×, except for the first row and the last panel of the last row (40×). Sections shown are representative of at least 10 individual sections and 5 independent experiments. (B) RORγt expression in DP thymocytes, spleen αβ T cells and intestinal lymphoid cells. Cells from Rorc(γt)^{+/GFP} adult mice (blue histograms) and control Rorc(γt)^{+/+} mice (red lines) were analyzed by flow cytometry for expression of GFP. Cells were gated as indicated. Lin⁻c-kit⁺IL-7Rα⁺ cells represented approximately 0.5% of total intestinal mononuclear cells and 0.1 to 0.2% of total PP cells. The data shown are representative of at least 10 individual mice. (C) Expression of c-kit and IL-7Rα by intestinal lin⁻ RORγt⁺ cells. Cells from Rorc(γt)^{+/GFP} adult mice were analyzed by flow cytometry and gated on lin⁻ cells. Numbers indicate the percent cells present in each quadrant. The data shown are representative of at least 10 individual mice.

FIG. 2. RORγt is required for the generation of lin⁻c-kit⁺IL-7Rα⁺ cells, CPs, and isolated lymphoid follicles (ILFs). (A) T cells and lin⁻ cells from the small intestine of RORγt-expressing (Rorc(γt)^{/GFP} or Rorc(γt)^{+/+}), designated as wt, and RORγt-deficient (Rorc(γt)^{GFP/GFP}) mice, designated as RORγt⁰ mice, were analyzed by flow cytometry. Numbers indicate the percent cells present in each quadrant. The data shown are representative of at least 10 individual mice. (B) Absolute numbers of B cells, T cell subsets, and lin⁻c-kit⁺IL-7Rα⁺ cells in the small intestine of RORγt-expressing (white bars), RORγt-deficient (black bars), and RORγt-deficient , Bcl-xL transgenic (grey bars) mice. DN/4, 8αβ and 8αα indicate the CD4⁻CD8⁻ and CD4⁺, the CD8αβ⁺ and the CD8αα⁺ subsets of αβ T cells, respectively. Fifteen Rorc(βt)^{+GFP} or Rorc(γt)^{+/+}mice, 10 Rorc(γt)^{GFP/GFP}, and 5 Rorc(γt)^{GFP/GFP}/Rorc(γt)-Bcl-xl^{α/GFP} mice were analyzed by flow cytometry. In statistical analyses using Student's t test, all groups are compared to the corresponding wild-type control (white bars). *p<10⁻², **p<10⁻³, ***p<10⁻⁵. In control groups (white bars), the number of αβ T cells may be over-estimated due to possible contamination from remaining PP cells. (C) Longitudinal sections of the small intestine of Rorc(γt) deficient mice were stained as indicated, as well as for GFP (green). Even though small clusters of hematopoietic (CD45⁺) cells were present, the absence of CD11c⁺ dendritic cell and B cell clusters suggests the absence of CPs and ILFs, respectively. Magnifications are 100× (first two panels) and 200× (last two panels). Sections shown are representative of at least 10 individual sections and 3 independent experiments.

FIG. 3. Cell-fate mapping of RORγe cells. (A) Strategy for genetic cell fate mapping. Rorc(γt)-Cre^{TG} mice express Cre under control of the Rorc(γt) locus on a BAC transgene. The Cre gene was inserted into the first exon of Rorc(γt). Cd4-Cre^{TG} mice express Cre under control of a short synthetic promoter consisting (from 5′ to 3′) of the murine CD4 proximal enhancer, promoter, exon 1, intron 1 containing the CD4 silencer, and part of exon 2. R26R mice express GFP under control of the Rosa26 locus only after Cre-mediated excision of a LoxP-flanked Stop sequence. The Rosa26 gene is expressed ubiquitously. (B) Cells from thymus, spleen and small intestine of adult Rorc(γt)-Cre^{TG}/R26R mice (blue histograms), from the small intestine of Cd4-Cre^{TG} I R26R mice (blue histograms) and from control R26R mice (red lines) were analyzed by flow cytometry for the expression of GFP. Cells were gated as indicated. The data shown are representative of 8 (Rorc(γt)-Cre^{TG}), 5 (CD4-Cre^{TG}) and 10 (R26R) individual mice.

FIG. 4. Normal cell cycle progression and in vitro survival of thymocytes from RORγt-deficient, Bcl-xL BAC-transgenic mice. Cell cycle analysis was performed by propidium iodide (PI) staining of fresh thymocytes isolated from Rorc(γt)-Bcl-xl^{TG} mice (Bcl^{TG}), Ror(γt)^{GFP/GFP} (RORγt°) and from RORγt°Bcl^{TG} mice. Numbers indicate the percent cells found in S+G2/M phase of the cell cycle. In vitro survival was evaluated by cultures of thymocytes for different periods of time and subsequent Annexin V staining of live cells. Similar results were obtained with Bcl^{TG} and wild-type mice. The data shown are representative of 3 individual experiments.

FIG. 5. Cell fate mapping of RORγt⁺ or CD4+ cells (A) Cells from thymus, spleen and intestine of adult Rorc(γt)-Cre^{TG}/R26R (blue histograms) or control R26R mouse (red lines), were analyzed by flow cytometry for the expression of GFP. Cells were gated as indicated. The data shown are representative of 3 individual experiments. (B) Expression of CD4 by intestinal lin⁻ RORγt⁺ cells. Numbers indicate the percent cells present in each quadrant. The data shown are representative of 3 individual experiments. (C) To demonstrate that the Rosa26 promoter is also active in B cells and γδ T cells, R26R mice were crossed to the ubiquitous deleter Tk-Cre^{TG} mouse line. Similar results were obtained with splenocytes. The data shown are representative of two independent experiments. (D) Splenocytes from Rag-2-deficient Rorc(γt)-Cre^{TG}/R26R mice (blue histograms) or Rag-2-deficient R26R mouse (red lines) were analyzed for the expression of GFP. Cells were gated as indicated. The data shown are representative of 3 individual mice.

FIG. 6. Absence of mature CPs and ILFs in LTα-deficient mice. Longitudinal sections of the small intestine of adult Lta^{-/-} Rorc(γt)^{+/GFP} mice were stained as indicated, as well as for GFP (green). In these mice, CP rudiments were found that consisted of small clusters of RORγt⁺ cells, but that contained very few CD11c+ dendritic cells. No ILFs were present. RORγt⁺ cells expressed low amounts of CD45, only apparent in these panels when the green fluorescence was removed. Magnifications are 100× (first two panels) and 200× (last two panels). Sections shown are representative of at least 10 individual sections and 3 individual mice.

FIG. 7. RORγt⁺ cells in the postnatal intestinal lamina propria. Longitudinal sections of the small intestine of Rorc(γt)^{+/GFP} mice at different times after birth were stained as indicated, as well as for EGFP (green). Magnification is 40×. Sections shown are representative of at least 5 individual sections and 2 independent experiments.

FIG. 8. RORγt⁺ T cells in the postnatal intestinal lamina propria: Surface Staining. The mouse used is heterozygous RORgt-GFP-KI. Lamina propria lymphocytes (LPLs) were isolated from small intestine and colon. Briefly, intestinal tubes were dissected out and after removal of Peyer's Patches the tubes were opened longitudinally and cut into 1.5 cm pieces. Epithelial cells and intraepithelial lymphocytes (IELs) were removed by treating with 5 mM EDTA. The pieces were then digested with 0.5 mg/ml of each of Collagenase D (Roche) and DNAse I (Sigma) as well as 0.5 U/ml Dispase (Fisher). LPLs were recovered by applying the digested intestine to a Percoll gradient (80:40). For the flow cytometry the following antibodies were used: anti-mouse CD3-PerCP (145-2C11) (BD Pharmingen), anti-TCRgd-PE (GL3) (BD Pharmingen), anti-TCRb-APC (H57-597) (BD Pharmingen). GFP fluorescence was detected directly.

FIG. 9. Identification of IL-17 Producing T cells from the small intestine of Rorc(γt)^{+/-} compared to Rorc(γt)^{-/-} and wild type mice: No Stimulation with PMA The mouse used is heterozygous RORγt-GFP-KI. The lamina propria lymphocytes (LPLs) are isolated from the small intestine by the method described in the legend from FIG. 8. The isolated LPLs were cultured in 96 well plates for 5 h (1×10⁶ cells per well) without any stimulation. The cells were surface stained with anti-mouse TCRb-APC (BD Pharmingen) and then fixed and permeabilized for intracellular cytokine staining with rat anti-mouse IL-17-PE (BD Pharmingen). The top panel shows the flow cytometry results in B6 WT controls, the second panel are the results from the RORγt^{+/-} mice, and panel three are the results from the RORγt^{-/-} mice.

FIG. 10. Identification of IL-17 Producing T cells from the small intestine of Rorc(γt)^{+/GFP} mice: Stimulation with PMA

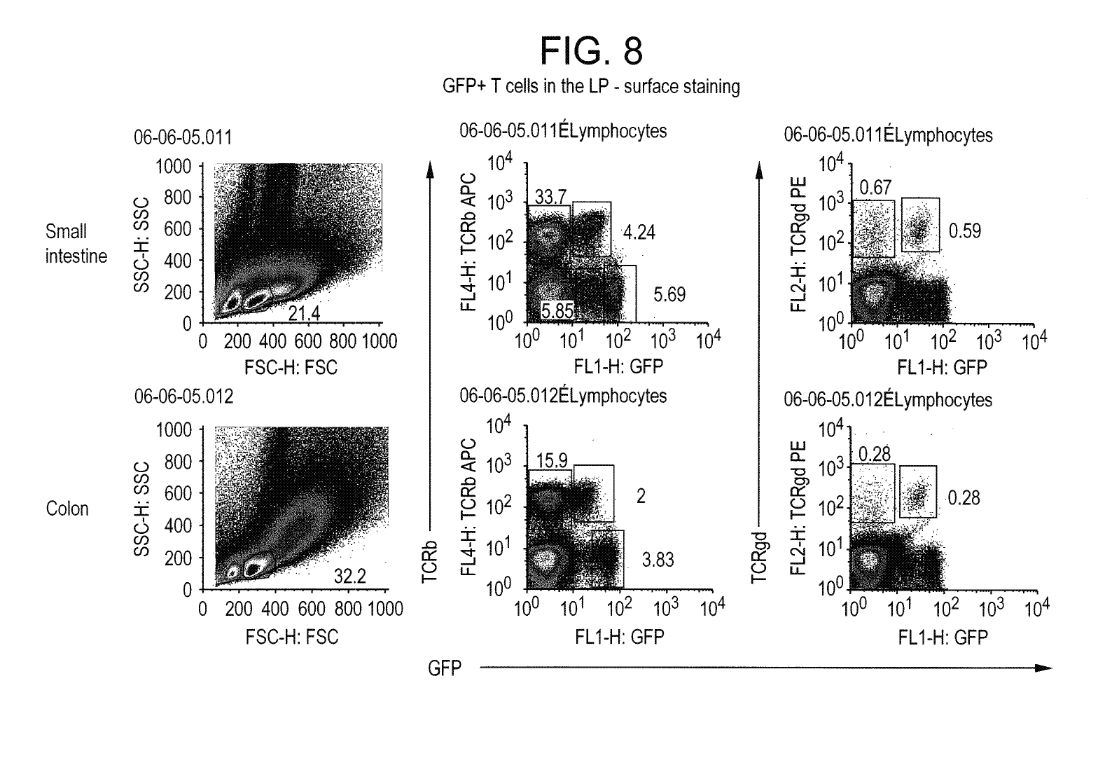

The mouse is heterozygous RORγt-GFP-KI. The lamina propria lymphocytes (LPLs) are isolated from the small intestine by the method described in the legend from FIG. 8. The isolated LPLs were cultured in 96 well plates for 5 h (1×10⁶ cells per well) without any stimulation or with PMA/Ionomycin (50 ng/ml PMA+200 ng/ml Ionomycin) or the wells were precoated with 5 ug/ml purified anti-CD3+anti-CD28 Abs in PBS for the CD3/CD28 stimulation. After the stimulation the cells were first surface stained with anti-mouse CD3-PerCP (BD Pharmingen) and anti-mouse TCRb-APC (BD Pharmingen) and then fixed and permeabilized for intracellular cytokine staining with rat anti-mouse IL-17-PE (BD Pharmingen). For the isotype controls one of the CD3/CD28 stimulated samples was stained with rat anti-mouse IgG1-PE (BD Pharmingen).

FIGS. 11A and 11B. RORγt Is Expressed in a Subpopulation of Lamina Propria T Cells. (A) Lamina propria lymphocytes (LPL) were isolated from the small intestine and colon of heterozygous RORγt-reporter mice (Rorγt^{gfp/+}) and stained for TCRβ and TCRγδ. Representative data from multiple experiments are shown. (B) Expression of RORγt (GFP) in the small intestinal lamina propria of heterozygous (Rorγt^{gfp/+}) and homozygous (Rorγt^{gfp/gfp}) RORγt-reporter mice.

FIGS. 12A and 12B. RORγt Is Required for the Generation of Lamina Propria IL-17⁺ T Cells. (A) RORγt⁺ (GFP⁺) T cells from Rorγt^{gfp/+} mice express IL-17. LPLs were stimulated in vitro with plate bound anti-CD3/anti-CD28 for 5 hours and in the presence of Brefeldin A for the final 2 hours, after which they were fixed and stained for intracellular IL-17 and GFP. (B) Comparison of lamina propria IL-17⁺ and IFNγ⁺ T cells in Rorγt^{gfp/+} and Rorγt^{gfp/gfp} mice. Freshly isolated LPLs from heterozygous (Rorγt^{gfp/+}) and homozygous null (Rorγt^{gfp/gfp}) mice were stimulated in vitro with PMA/Ionomycin for 5 hours or incubated with Brefeldin A only (no PMA/Iono) and stained for CD4, TCRβ, and intracellular cytokines. The gating used is shown in FIG. S2. Representative data from multiple experiments are shown.

FIGS. 13A, 13B and 13C. In Vitro Differentiation of Th17 Cells Requires RORγt. (A) Cytokine production by naive CD4⁺CD25⁻CD62⁺CD44⁻ T cells from WT and Rory^{-/-} mice after stimulation with anti-CD3/CD28 for 3 days with or without TGF-β and IL-6. (B) Time course of RORγt, IL-17, and IL-17F mRNA expression following stimulation as in (A). (C) RORγt, IL-17, and IL-17F mRNA expression following stimulation as in (A) at the 48 hour time point. Relative expression levels were measured by quantitative real-time RT-PCR and were normalized to actin expression level using the Standard Curve Method.

FIGS. 14A, 14B and 14C. Induction of IL-17 in CD4⁺ T Cells by Ectopic Expression of RORγt. (A) IL-17 and IFNγ production in MACS-sorted CD4⁺ T cells isolated from wild-type C57BL/6 and Balb/c mice and transduced with retroviral vectors encoding IRES-GFP (MIG), T-bet-IRES-GFP (T-bet), and RORγt -IRES-GFP (RORγt). Cells were analyzed after 5 days in culture. (B) Relative expression levels of IL-17 and IL-17F mRNAs in retrovirally transduced MACS-sorted CD4⁺ T cells. Expression levels were monitored by quantitative real-time RT-PCR, and data were normalized to GAPDH expression level using the Standard Curve Method. (C) IL-17 production in sorted naïve CD4⁺ T cells (CD4⁺CD25⁻CD62⁺CD44⁻) transduced with retroviral constructs encoding IRES-GFP (MIG) and RORγt-IRES-GFP (RORγt).

FIGS. 15A and 15B. IL-6 Controls the Differentiation of RORγt⁺ Th17 Cells in the Lamina Propria. (A) Comparison of IL-17⁺CD4⁺ T cells in the lamina propria of WT (B6) and IL-6-deficient mice. LPLs were isolated from two 17-week-old mice from each genotype and stimulated for 4 h with PMA/Ionomycin in the presence of Brefeldin A. (B) Levels of RORγt, IL-23R, IL-17, and IL-17F mRNA expression in sorted TCR ⁺CD4⁺ lamina propria T cells from WT and IL-6-deficient mice.

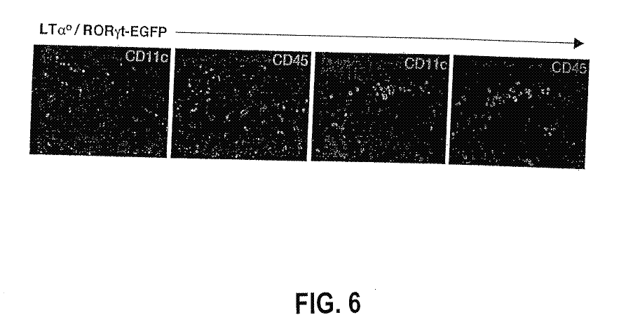

FIGS. 16A, 16B, 16C, 16D and 16E. Reduced Severity of EAE and Absence of Infiltrating Th17 Cells in Mice with RORγt-Deficient T Cells. (A) EAE disease course in wild-type and Rorγ^{-/-} mice (data are from 6 C57BL/6 and 3 syngeneic Rorγ^{-/-} mice). (B) EAE disease course in RAG2-deficient mice reconstituted with MACS purified CD4⁺ splenocytes from wild-type (WT) and Rorγ^{-/-} mice. Data are representative of two independent experiments—n=5 (WT), n=3 (Rorγ^{-/-}). (C) EAE disease course in RAG2-deficient mice reconstituted with total bone marrow cells from wild-type or Rorγ^{-/-} animals (5 animals in each group). All recipient animals were irradiated with a sublethal dose (400 rads/animal) before reconstitution, and EAE was induced 11 weeks later. To assess similar reconstitution efficiency, blood before disease induction as well as spleen populations on day 21 after induction were compared between the two groups (FIG. S6 and data not shown). Data are representative of 3 independent experiments. (D,E) Cytokine production by lymphocytes isolated on day 21 after disease induction from the spinal cords of RAG-deficient mice reconstituted with WT and Rorγ^{-/-} bone marrow (experiment in FIG. 6C). The cells were stimulated for 4 hours with PMA/Ionomycin and stained for surface markers and intracellulat cytokines. Representative FACS plots (gated on TCR ⁺CD4⁺ cells) from mice from each group are shown in panel D. Clinical scores are shown in parentheses. In the Rorγ^{-/-} group, 3 out of 5 mice did not develop any clinical signs of disease (score 0), but all had considerable spinal cord infiltrate. One is shown in panel D. Similar results were achieved in 3 independent experiments. Tabulated results from all mice are presented in panel E as percentage of TCRβ⁺CD4⁺ cells in the spinal cord infiltrate. Total: all IL-17⁺ cells; IFNγ⁻: IL-17⁺IFNγ⁺ cells; IFNγ⁻: IL-17⁺IFNγ⁻ cells; CD4⁺IFNγ⁺: IL-17⁻IFNγ⁺ cells. ** p=0.002, * p=0.006, unpaired t test.

FIG. 17. Model of Th17 Development in the Intestinal Lamina Propria. Th17 development in the gut requires RORγt expression in CD4⁺ T cells. RORγt expression results from the action of IL-6 and TGF-β (but not IL-23) produced by activated dendritic cells (DCs) and other cells in the lamina propria. DCs can be activated by signals derived from the luminal flora or infectious agents and TLR ligands that gain access to the lamina propria. It is currently unknown if IL-6, TGF-β and IL-23 are produced by different types of DCs or by the same DC. TGF-β may also be derived from regulatory T cells (Tregs), which normally suppress Th1 and Th2 cell development. IL-6 may also inhibit TGF-β -induced differentiation of Tregs, thus further promoting Th17 development. RORγt⁺ T cells upregulate IL-23R and thus become responsive to IL-23. IL-23 reinforces the Th17 phenotype by possibly helping in maintenance, expansion or further differentiation of the cells.

FIG. 18. Intraepithelial Lymphocytes Do Not Express RORγ t. LPLs and IELs were isolated from the small intestines of heterozygous (Rorγt^{gfp+}) and homozygous (Rorγt^{gfp/gfp}) RORyt-reporter mice. In the lamina propria of homozygous (knock-out) mice the complete disappearance of CD4hiGFP+ and CD4⁻GFP+ cryptopatch or lymphoid tissue inducer-like cells is evident.

FIG. 19. Comparison of GFP Expression in LP cells of Rorγt^{gfp+} and Rorγt^{gfp/gfp} Mice. Surface staining and GFP expression are displayed for the cells shown in FIG. 12B. GFP expression was detected in both CD4+ and CD4− T cells.

FIG. 20. Local IL-17 Expression in the Lamina Propria. LPLs were isolated from small intestine, cecum, colon, and rectum of 12-week-old WT C57BL16 mice. The cells were stimulated for 4 hours with PMA/Ionomycin in the presence of Brefeldin A and stained for surface markers and intracellular IL-17.

FIG. 21. Presence of IL-17+ Cells in the Lamina Propria is MyD88-Independent. LPLs were isolated from the small intestines of heterozygous (Myd88^{+/-}) and homozygous (Myd88^{-/-}) MyD88-deficient mice, stimulated for 4 hours with PMA/Ionomycin in the presence of Brefeldin A, and stained for surface markers and intracellular IL-17. Lower panels are gated on TCRβ+ cells.

FIG. 22. RORγ-Deficient Cells Can Undergo Normal Th1 Polarization in Vitro. 1.5×10⁶ MACS purified CD4+ splenocytes from WT and Rorγ^{-/-} mice were stimulated for 3 days with plate bound anti-CD3 and anti-CD28 in the presence or absence of 20 ng/ml IL-12 and then rested for 3 days in the presence of 40 units/ml IL-2 and in the presence or absence of IL-12. Surface and intracellular cytokine staining was performed on day 6. Plots are gated on CD4+ cells.

FIG. 23. Reconstitution Efficiency and Spinal Cord Infiltrate in Bone Marrow Chimeras. Surface staining of spleens and spinal cord infiltrates of mice reconstituted with WT or RORγ-deficient bone marrow. Representative data from two mice from day 21 post disease induction. Clinical scores are in parenthesis.

FIG. 24. Lack of IL-17+ MOG-specific T cells in Draining Lymph Nodes. 4×10⁶ total draining lymph node cells were isolated from mice reconstituted with WT or RORγt-deficient bone marrow at day 21 post EAE induction. The total number of CD4+ T cells was similar between the two groups (data not shown, text and FIG. S6). The cells were stimulated in vitro in the presence of 40 pg/ml MOG 35-55 peptide and 20 ng/ml IL-23 for 4 days and the frequency of IFNγ+ and IL-17+ MOG-specific T cells assessed by flow cytometry. Although similar numbers of CD4+ T cells were recovered in both groups, IL-17+ cells were reduced to background levels in the absence of RORγt.

FIG. 25. RORγt Is Required for Pathogenic Th17 Responses. Lymphocytes were isolated from the spinal cords of Rag2^{-/-} mice reconstituted with CD4+ T cells from wildtype(WT) and Rorγ^{-/-} spleens on day 24 post EAE induction (experiment shown in FIG. 15B). The cells were stimulated for 4 hours with PMA/Ionomycin in the presence of Brefeldin A and stained for surface markers and intracellular cytokines. Plots are gated on TCRβ+ cells.

FIG. 26. Shows that the ligand binding domain/AF2 can be substituted with VP16 activation domain to induce IL-17 production.

FIG. 27. Shows that the A304V mutant (conservative mutation) can still down-regulate expression of CD8 in the cell line, but not as well as the wild-type protein.

FIG. 28. Shows that the bulky substitution A304F in the ligand binding pocket results in loss of induction of IL-17, as does truncation of N and C-terminal sequences. However, the conservative change A304V has little effect.

FIG. 29. Shows that RORγt expression/activation also down-regulates interferon-gamma production as shown here with wt and A304V mutant. Others are inactive.

FIG. 30. This is a control showing expression levels of different mutant and wild type RORγt in the transduced T cells—note that N2 and C2 are not well expressed, but key are the A304 point mutants.

FIG. 31. Shows that RORγt -KO CD4+ T cells do not cause colitis

FIG. 32. Shows increased Th17 numbers in colon of colitic WT mice

FIG. 33. Shows the nucleic acid sequence for human RORγt (SEQ ID NO: 1)

FIG. 34. Shows the amino acid sequence for human RORγt (SEQ ID NO: 2)

FIG. 35. Shows the nucleic acid sequence for mouse RORγt (SEQ ID NO: 3)

FIG. 36. Shows the amino acid sequence for mouse RORγt (SEQ ID NO: 4)

FIG. 37. Shows the nucleic acid sequence for human RORγ (SEQ ID NO: 5)

FIG. 38. Shows the amino acid sequence for human RORγ (SEQ ID NO: 6)

FIG. 39. Shows the nucleic acid sequence for mouse RORγ (SEQ ID NO: 7)

FIG. 40. Shows the amino acid sequence for mouse RORγ (SEQ ID NO: 8)

FIG. 41. Shows the two DNA sequences from which two shRNA molecules were prepared and tested for their ability to inhibit expression of RORγt.

FIG. 42. Delivery of siRNA Hairpins by a Retroviral vector. The top panel shows the shRNA construct used for studies on inhibition of RORγt expression or function in cells. The bottom panel shows the results of a study whereby AKR1 cells were infected with a retrovirus encoding either the construct shown in the top panel, ie. the mRORγt-specific shRNA (R) or an empty retroviral vector (γt). After the AKR1 cells were infected with these vectors, mRORγt expression was monitored by Western blot. HMG-1 was used as an internal loading control. The results show that the shRNA specific for RORγt was effective in preventing expression of the RORγt gene. The effect lasted for at least two to three weeks, as shown in the lower panel.

FIG. 43. GRCA Expression is Altered by RORγt Overexpression or RNAi Knockdown. A study was done to determine whether the shRNA described in FIG. 42 could be effective at blocking the expression of a target gene for RORγt, which is a G protein coupled receptor GRCA (Gene Rich Cluster A gene). The results show that when compared to the overexpression of RORγt shown in the AKR1 cells in the top panel, cells transfected with the shRNA showed significant reduction in the level of GRCA, in the lower panel. Thus, this is support that a shRNA specific for RORγt can block the expression, not only of RORγt, but also of a target protein of RORγt.

### DETAILED DESCRIPTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated by reference in their entireties.

### Definitions

As noted above, the terms used herein have the meanings recognized and known to those of skill in the art. However, for convenience and completeness, particular terms and their meanings are set forth below.

The following terms are used to describe certain immune cells or structures studied herein. For example, the term “DP or double positive thymocytes” are immature thymocytes that express both the CD4 and CD8 receptors on their surface. “Isolated lymphoid follicles” or “ILF” are also known as lymphoid nodules. In the colon, “isolated lymphoid follicles” are known as colon patches or “CP”. “Intraepithelial lymphocytes” as used herein refers to T cells located in the lining of the intestine. These T cells, also referred to as “IEL” play key roles in protecting the body from invasion by harmful bacteria and viruses, minimizing immune responses to food and harmless bacteria and in promoting the repair of the intestinal lining.

“Cryptopatch (CP) cells” are unique cell clusters found in the bowel wall. These small clusters of hematopoietic cells have been detected between crypts in the wall of the small intestine.

“Inflammatory bowel disease” (IBD) can involve either or both the small and large bowel. Crohn's disease and ulcerative colitis are the best known forms of IBD, and both fall into the category of “idiopathic” inflammatory bowel disease because the etiology for them is unknown. Pathologic findings are generally not specific, although they may suggest a particular form of IBD. “Active” IBD is characterized by acute inflammation. “Chronic” IBD is characterized by architectural changes of crypt distortion and scarring. Crypt abscesses (active IBD consisting of neutrophils in crypt lumens) can occur in many forms of IBD, not just ulcerative colitis.

“Anti-tumor immunity” refers to an immune response that has been generated to a specific tumor cell or to specific cancerous tissue. The response may be either a B cell (antibody) response or it may be a T cell (cell-mediated) response.

The term “immunogen” is used herein to describe a composition typically containing a peptide or protein, or a glycolipid as an active ingredient (i.e., antigen) used for the preparation of antibodies against the peptide or protein or the glycolipid or for eliciting a T cell response.

The term “immunogenic” refers to the ability of an antigen to elicit an immune response, either humoral or cell mediated. An “immunogenically effective amount” as used herein refers to the amount of antigen sufficient to elicit an immune response, either a cellular (T cell) or humoral (B cell or antibody) response, as measured by standard assays known to one skilled in the art. The effectiveness of an antigen as an immunogen, can be measured either by proliferation assays, by cytolytic assays, such as chromium release assays to measure the ability of a T cell to lyse its specific target cell, or by measuring the levels of B cell activity by measuring the levels of circulating antibodies specific for the antigen in serum, or by measuring the number of antigen specific colony forming units in the spleen. Furthermore, the level of protection of the immune response may be measured by challenging the immunized host with the antigen-bearing pathogen. For example, if the antigen to which an immune response is desired is a virus or a tumor cell, the level of protection induced by the “immunogenically effective amount” of the antigen is measured by detecting the level of survival after virus or tumor cell challenge of the animals.

The term “mucosal immunity” refers to resistance to infection across the mucous membranes. Mucosal immunity depends on immune cells and antibodies present in the linings of reproductive tract, gastrointestinal tract and other moist surfaces of the body exposed to the outside world. Thus, a person having mucosal immunity is not susceptible to the pathogenic effects of foreign microorganisms or antigenic substances as a result of antibody secretions of the mucous membranes. Mucosal epithelia in the gastrointestinal, respiratory, and reproductive tracts produce a form of IgA (IgA, secretory) that serves to protect these ports of entry into the body. Since many pathogens enter the host by way of the mucosal surfaces, a vaccine that elicits mucosal immunity would be beneficial in terms of protection from many known pathogens, such as influenza or SARS virus. Furthermore, it is known that T cell tolerance to specific antigens can be established by administering the antigen via the oral route, thus representing a mechanism to prevent inflammation in response to commensal bacteria, food components, etc. Accordingly, there may be a potential role for RORγt-expressing cryptopatch cells in the process of induction of oral tolerance.

“Subunit vaccines” are cell-free vaccine prepared from purified antigenic components of pathogenic microorganisms, thus carrying less risk of adverse reactions than whole-cell preparations. These vaccines are made from purified proteins or polysaccharides derived from bacteria or viruses. They include such components as toxins and cell surface molecules involved in attachment or invasion of the pathogen to the host cell. These isolated proteins act as target proteins/antigens against which an immune response may be mounted. The proteins selected for a subunit vaccine are normally displayed on the cell surface of the pathogen, such that when the subject's immune system is subsequently challenged by the pathogen, it recognizes and mounts an immune reaction to the cell surface protein and, by extension, the attached pathogen. Because subunit vaccines are not whole infective agents, they are incapable of becoming infective. Thus, they present no risk of undesirable virulent infectivity, a significant drawback associated with other types of vaccines. Subunit molecules from two or more pathogens are often mixed together to form combination vaccines. The advantages to combination vaccines is that they are generally less expensive, require fewer inoculations, and, therefore, are less traumatic to the animal.

A “DNA vaccine” relates to the use of genetic material (e.g., nucleic acid sequences) as immunizing agents. In one aspect, the present invention relates to the introduction of exogenous or foreign DNA molecules into an individual's tissues or cells, wherein these molecules encode an exogenous protein capable of eliciting an immune response to the protein. The exogenous nucleic acid sequences may be introduced alone or in the context of an expression vector wherein the sequences are operably linked to promoters and/or enhancers capable of regulating the expression of the encoded proteins. The introduction of exogenous nucleic acid sequences may be performed in the presence of a cell stimulating agent capable of enhancing the uptake or incorporation of the nucleic acid sequences into a cell. Such exogenous nucleic acid sequences may be administered in a composition comprising a biologically compatible or pharmaceutically acceptable carrier. The exogenous nucleic acid sequences may be administered by a variety of means, as described herein, and well known in the art. The DNA is linked to regulatory elements necessary for expression in the cells of the individual. Regulatory elements include a promoter and a polyadenylation signal. Other elements known to skilled artisans may also be included in genetic constructs of the invention, depending on the application. The following references pertain to methods for the direct introduction of nucleic acid sequences into a living animal: Nabel et al., (1990) Science 249:1285-1288; Wolfe et al., (1990) Science 247:1465-1468; Acsadi et al. (1991) Nature 352:815-818; Wolfe et al. (1991) BioTechniques 11(4):474-485; and Felgner and Rhodes, (1991) Nature 349:351-352, which are incorporated herein by reference. Such methods may be used to elicit immunity to a pathogen, absent the risk of infecting an individual with the pathogen. The present invention may be practiced using procedures known in the art, such as those described in PCT International Application Number PCT/US90/01515, wherein methods for immunizing an individual against pathogen infection by directly injecting polynucleotides into the individual's cells in a single step procedure are presented, and in U.S. Pat. Nos. 6,635,624; 6,586,409; 6,413,942; 6,406,705; 6,383,496.

An “agonist” is an endogenous substance or a drug that can interact with a receptor and initiate a physiological or a pharmacological response characteristic of that receptor (contraction, relaxation, secretion, enzyme activation, etc.). An agonist has a positive intrinsic activity. “Intrinsic activity” is the ability of a drug (and cell) to transduce a drug-receptor binding event into a biological response.

An “antagonist” or “inhibitor” is a substance such as a small organic molecule or a protein or peptide or nucleic acid molecule such as an antisense nucleic acid or a small interfering RNA molecule (siRNA or shRNA) or an antibody that prevents the expression and/or function of a designated molecule, such as in the matter of the present invention, the molecule is RORγt (SEQ ID NOs: 1 and 2, human nucleic acid and amino acid sequences for RORγt, respectively).

“Lamina propria” is loose connective tissue in a mucosa. Lamina propria supports the delicate mucosal epithelium, allows the epithelium to move freely with respect to deeper structures, and provides for immune defense. Compared to other loose connective tissue, lamina propria is relatively cellular. It has been called “connective tissue with lymphatic tendencies”. Because mucosal epithelium is relatively delicate and vulnerable (i.e., rather easily breached by potential invading microorganisms, compared to epidermis), lamina propria contains numerous cells with immune function to provide an effective secondary line of defense. Lymphoid tissue occurs in lamina propria all along the GI tract, where it is sometimes referred to as “GALT”, for “Gut-Associated Lymphoid Tissue”. The most characteristic feature of gut-associate lymphoid tissue is the presence of clusters of lymph nodules (also called lymphoid follicles), which are sites where lymphocytes congregate. At the center of each lymph nodule is a germinal center where the lymphocytes proliferate.

“Tertiary lymphoid organs” are lymphoid tissues that develop in response to inflammatory stimuli, in contrast to secondary lymphoid organs, such as lymph nodes and Peyer's patches, that develop in the fetus following a developmental program. Tertiary lymphoid tissues are commonly found in chronically inflamed tissues that are the target of autoimmunity, such as in reumathoid arthritis, thyroiditis, and type I diabetes.

As used herein a “small organic molecule” is an organic compound (or organic compound complexed with an inorganic compound (e.g., metal)) that has a molecular weight of less than 3 kilodaltons, and preferably less than 1.5 kilodaltons.

As used herein a “reporter”gene is used interchangeably with the term “marker gene” and is a nucleic acid that is readily detectable and/or encodes a gene product that is readily detectable such as green fluorescent protein (as described in U.S. Pat. No. 5,625,048 issued Apr. 29, 1997, and WO 97/26333, published Jul. 24, 1997, the disclosures of each are hereby incorporated by reference herein in their entireties) or luciferase.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The phrase “therapeutically effective amount” is used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in one or more clinically significant symptoms in the host.

“Agent” refers to all materials that may be used to prepare pharmaceutical and diagnostic compositions, or that may be compounds, such as small synthetic or naturally occurring organic compounds, nucleic acids, polypeptides, antibodies, fragments, isoforms, variants, or other materials that may be used independently for such purposes, all in accordance with the present invention.

“Treatment” or “treating” refers to therapy, prevention and prophylaxis and particularly refers to the administration of medicine or the performance of medical procedures with respect to a patient, for either prophylaxis (prevention) or to reduce the extent of or likelihood of occurrence of the infirmity or malady or condition or event in the instance where the patient is afflicted. It also refers to reduction in the severity of one or more symptoms associated with the disease or condition. In the manner of the present application, it may refer to amelioration of one or more of the following: pain, swelling, redness or inflammation associated with an inflammatory condition or an autoimmune disease.

“Diagnosis” or “screening”refers to diagnosis, prognosis, monitoring, characterizing, selecting patients, including participants in clinical trials, and identifying patients at risk for or having a particular disorder or clinical event or those most likely to respond to a particular therapeutic treatment, or for assessing or monitoring a patient's response to a particular therapeutic treatment.

“Subject” or “patient” refers to a mammal, preferably a human, in need of treatment for a condition, disorder or disease.

As used herein, the terms “nucleic acid”, “polynucleotide” and “oligonucleotide” refer to primers, probes, and oligomer fragments to be detected, and shall be generic to polydeoxyribonucleotides (containing 2-deoxy-D-ribose), to polyribonucleotides (containing D-ribose), and to any other type of polynucleotide which is an N-glycoside of a purine or pyrimidine base, or modified purine or pyrimidine bases (including abasic sites). There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single-stranded RNA.

The “polymerase chain reaction (PCR)” technique, is disclosed in U.S. Pat. Nos. 4,683,202, 4,683,195 and 4,800,159. In its simplest form, PCR is an in vitro method for the enzymatic synthesis of specific DNA sequences, using two oligonucleotide primers that hybridize to opposite strands and flank the region of interest in the target DNA. A repetitive series of reaction steps involving template denaturation, primer annealing and the extension of the annealed primers by DNA polymerase results in the exponential accumulation of a specific fragment (i.e, an amplicon) whose termini are defined by the 5′ ends of the primers. PCR is reported to be capable of producing a selective enrichment of a specific DNA sequence by a factor of 10⁹. The PCR method is also described in Saiki et al., 1985, Science, 230:1350.

As used herein, “probe” refers to a labeled oligonucleotide primer, which forms a duplex structure with a sequence in the target nucleic acid, due to complementarity of at least one sequence in the probe with a sequence in the target region. Such probes are useful for identification of a target nucleic acid sequence for ROR gamma t according to the invention. Pairs of single-stranded DNA primers can be annealed to sequences within a target nucleic acid sequence or can be used to prime DNA synthesis of a target nucleic acid sequence.

By “homologous” is meant a same sense nucleic acid which possesses a level of similarity with the target nucleic acid within reason and within standards known and accepted in the art. With regard to PCR, the term “homologous” may be used to refer to an amplicon that exhibits a high level of nucleic acid similarity to another nucleic acid, e.g., the template cDNA. As is understood in the art, enzymatic transcription has measurable and well known error rates (depending on the specific enzyme used), thus within the limits of transcriptional accuracy using the modes described herein, in that a skilled practitioner would understand that fidelity of enzymatic complementary strand synthesis is not absolute and that the amplified nucleic acid (i.e., amplicon) need not be completely identical in every nucleotide to the template nucleic acid.

“Complementary” is understood in its recognized meaning as identifying a nucleotide in one sequence that hybridizes (anneals) to a nucleotide in another sequence according to the rule AγT, U and CγG (and vice versa) and thus “matches” its partner for purposes of this definition. Enzymatic transcription has measurable and well known error rates (depending on the specific enzyme used), thus within the limits of transcriptional accuracy using the modes described herein, in that a skilled practitioner would understand that fidelity of enzymatic complementary strand synthesis is not absolute and that the amplicon need not be completely matched in every nucleotide to the target or template RNA. Thus, a sequence “complementary” to a portion of an RNA, as referred to herein, means a sequence having sufficient complementarity to be able to hybridize with the non-poly A portion of the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches it may comprise and still form a stable duplex (or triplex, as the case may be).

Procedures using conditions of high stringency are as follows. Prehybridization of filters containing DNA is carried out for 8 h to overnight at 65° C. in buffer composed of 6×SSC, 50 mM Tris-HC1 (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency that may be used are well known in the art. (see, e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, 1987-1997 Current Protocols,© 1994-1997 John Wiley and Sons, Inc.)

The term “antibody” as used herein includes intact molecules as well as fragments thereof, such as Fab and F(ab′)₂, which are capable of binding the epitopic determinant. Antibodies that bind the genes or gene products of the present invention can be prepared using intact polynucleotides or polypeptides or fragments containing small peptides of interest as the immunizing antigen attached to a carrier molecule. Commonly used carriers that are chemically coupled to peptides include bovine serum albumin and thyroglobulin. The coupled peptide is then used to immunize the animal (e.g, a mouse, rat or rabbit). The antibody may be a “chimeric antibody”, which refers to a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397.). The antibody may be a human or a humanized antibody. The antibody may be prepared in mice, rats, goats, sheep, swine, dogs, cats, or horses.

“Gene Product” as used herein, unless otherwise indicated, is a protein or polypeptide encoded by the nucleic acid sequences identified by the methods of the present invention, or a nucleic acid comprising a sequence hybridizable to these sequences, or their complement under conditions of high stringency, or a protein comprising a sequence encoded by said hybridizable sequence; a nucleic acid at least 90% homologous to these sequences or their complement as determined using the NBLAST algorithm; a nucleic acid at least 90% homologous to these sequences, or a fragment or derivative of any of the foregoing proteins or nucleic acids.

“Candidate compound” or “test compound” refers to any compound or molecule that is to be tested, and more particularly for the present invention, for its ability to modulate RORγt expression or function. In addition, the “candidate compound” or “test compound” may be tested for its ability to increase or decrease the expression and/or function of one or more proinflammatory cytokines, or cytokine receptors, or one or more proinflammatory chemokines or chemokine receptors. As used herein, the terms, which are used interchangeably, refer to biological or chemical compounds such as simple or complex organic or inorganic molecules, peptides, proteins, peptidomimetics, peptide mimics, antibodies, nucleic acids (DNA or RNA), including oligonucleotides, polynucleotides, antisense molecules, small interfering nucleic acid molecules, such as siRNA or shRNA molecules, carbohydrates, lipoproteins, lipids, small molecules and other drugs.

The term “modulate” or “modulation”, as used herein, refers to either an increase or a decrease in the expression and/or activity or function of RORγt. Thus, a “modulator of RORγt” is defined as an agent that acts as an agonist or stimulator, which enhances expression and/or activity/function of RORγt, or an antagonist, which decreases expression and/or activity/function of RORγt. The activity or function of RORγt, as described herein, relates primarily to its effects on immune homeostasis. More particularly, the activity or function of RORγt, as described in the work presented herein, relates to its effects on mucosal immunity and its effects on proinflammatory cytokines, chemokines and their respective receptors.

Thus, the term “percent identical” or “percent sequence identity” refers to sequence identity between two amino acid sequences or between two nucleotide sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

RNA interference (RNAi) is an evolutionarily conserved mechanism in plant and animal cells that directs the degradation of messenger RNAs homologous to short double-stranded RNAs termed “small interfering RNA” or “siRNA” or “short hairpin RNA” or “shRNA”. The ability of siRNA to direct gene silencing in mammalian cells has raised the possibility that siRNA might be used to investigate gene function in a high throughput fashion or to modulate gene expression in human diseases. Methods of preparing siRNAs are known to those skilled in the art. The following references are incorporated herein by reference in their entirety: Reich et al., Mol Vis. 9:210-6 (2003); Gonzalez-Alegre P et al., Ann Neurol. 53:781-7 (2003); Miller et al., Proc Natl Acad Sci USA. (2003); Bidere et al., J Biol Chem., published as manuscript M301911200 (Jun. 2, 2003); Van De Wetering et al., EMBO Rep. 4:609-15 (2003); Miller and Grollman, DNA Repair (Amst) 2:759-63 (2003); Kawakami et al., Nat Cell Biol. 5:513-9 (2003); Abdelrahim et al., Mol Pharmacol. 63:1373-81 (2003); Williams et al., J Immunol. 170:5354-8 (2003); Daude et al., J Cell Sci. 116:2775-9 (2003); Jackson et al., Nat Biotechnol. 21:635-7 (2003); Dillin, Proc Natl Acad Sci USA. 100:6289-91 (2003); Matta et al., Cancer Biol Ther. 2:206-10 (2003); Wohlbold et al., Blood. (2003); Julien and Herr, EMBO J. 22:2360-9 (2003); Scherr et al., Cell Cycle. 2:251-7 (2003); Giri et al., J Immunol. 170:5281-94 (2003); Liu and Erikson, Proc Natl Acad Sci USA. 100:5789-94 (2003); Chi et al., Proc Natl Acad Sci USA. 100:6343-6 (2003); Hall and Alexander, J Virol. 77:6066-9 (2003).

“Antisense” nucleic acids are DNA or RNA molecules that are complementary to at least a portion of a specific mRNA molecule (See Weintraub, Sci. Amer. 262:40-46 (1990); Marcus-Sekura, Nucl. Acid Res, 15: 5749-5763 (1987); Marcus-Sekura Anal. Biochem., 172:289-295 (1988); Brysch et al., Cell Mol. Neurobiol., 14:557-568 (1994)). In the cell, the single stranded antisense molecule hybridizes to that mRNA, forming a double stranded molecule. The cell does not translate an mRNA in this double-stranded form. Therefore, antisense nucleic acids interfere with the expression of mRNA into protein. Oligomers of greater than about fifteen nucleotides and molecules that hybridize to the AUG initiation codon will be particularly efficient. Antisense methods have been used to inhibit the expression of many genes in vitro (Marcus-Sekura, Anal. Biochem., 172:289-295 (1988); Hambor et al., Proc. Natl. Acad. Sci. U.S.A. 85:4010-4014 (1988)) and in situ (Arima et al., Antisense Nucl. Acid Drug Dev. 8:319-327 (1998); Hou et al., Antisense Nucl. Acid Drug Dev. 8:295-308 (1998)).

### General Description

T lymphocytes are a subset of lymphocytes defined by their development in the thymus and expression of a T cell receptor (TCR; αβ or γδ heterodimers). T lymphocytes do not directly recognize pathogens, but MHC /peptide complexes expressed on antigen presenting cells (APC). T lymphocytes can be characterized by the expression of CD3 (part of the TCR complex) and can be subdivided into two major classes by the expression of either CD4 or CD8. CD4+ T lymphocytes recognize class II MHC/peptide complexes whereas CD8+ T lymphocytes are restricted to class I MHC/peptide complexes. T cells have receptors on their surfaces which allow it to interact with other cells and proteins. The T-cell receptor (TCR) is either gamma-delta or alpha-beta heterodimer. About 95% of all T-cells will express the alpha-beta TCR. The remainder express the gamma-delta TCR. In the normal development of T-cells, the gamma-delta TCR occurs first. T-cells expressing this receptor have cytotoxic capabilities and secrete recruiting lymphokines.

The majority of mature T lymphocytes fall into one of two functional categories: helper cells, which react with peptides complexed to major histocompatibility complex (MHC) class II molecules on antigen-presenting cells, and cytotoxic cells, which recognize peptides bound to MHC class I molecules. These cells are distinguished on the basis of surface expression of the CD4 or CD8 coreceptors, which are coexpressed on immature double-positive (DP) thymocytes but are singly expressed upon maturation. Cells that have T cell antigen receptors (TCRs) for self-MHC class I molecules express CD8, and cells with receptors for MHC class II express CD4. CD4 and CD8 bind to nonpolymorphic regions of class II and class I, respectively, and signal through their association with the cytoplasmic protein-tyrosine kinase Lck.

Mature T cells express either CD4 or CD8 on their surface. Most helper T cells express CD4, which binds to class II major histocompatibility complex (MHC) proteins, and most cytotoxic T cells express CD8, which binds to class I MHC proteins. In the thymus, mature CD4⁺CD8⁻ and CD4⁻ CD8⁺ T cells expressing αβ T-cell antigen receptors (TCR) develop from immature thymocytes through CD4⁺CD8⁺ αβ TCR⁺ intermediates.

Gamma/delta T cells differ from alpha/beta T cells in several ways:
Their TCR is encoded by different gene segments.
Their TCR binds to antigens that can be:
   intact proteins as well as a variety of other types of organic molecules (often containing phosphorus atoms).
   not “presented” within class I or class II histocompatibility molecules;
   not presented by “professional” antigen-presenting cells (APCs) like macrophages.
In the gut, IEL are mostly CD8αα homodimers.
Gamma/Delta T cells, like alpha/beta T cells, develop in the thymus. However, they migrate from there into body tissues, especially epithelia (e.g., intestine, skin, lining of the vagina), and don't recirculate between blood and lymph nodes. In man, gamma/delta T cells can make up to 30% of the blood T cells. They encounter antigens on the surface of the epithelial cells that surround them rather than relying on the APCs found in lymph nodes.

Situated as they are at the interfaces between the external and internal worlds, γδ T cells may represent a first line of defense against invading pathogens. Their response does seem to be quicker than that of αβ T cells.

CD8 consists of two polypeptide chains, α and β, of the Ig superfamily. Cell surface-expressed CD8 exists as either αβ heterodimers or αα homodimers. Thymus-derived CD8⁺ CTL generally express the CD8 αβ heterodimer, and the binding of CD8 to MHC class I is thought to strengthen the antigen-specific binding of the TCR to the peptide/MHC class I complex. However, the CD8αα homodimer is sufficient for binding to MHC class I. The CD8-alpha-alpha receptor protein appears to mediate the survival and differentiation of precursor cells into memory T cells and the homing or survival of IELs in the intestinal epithelium.

RORγt is expressed in double positive (CD4⁺CD8⁺) thymocytes, extending their survival during clonal selection, and in the LTi and LTi-like cells (Eberl, G., Marmon, S., Sunshine, M. J., Rennert, P. D., Choi, Y., and Littman, D. R. (2004). An essential function for the nuclear receptor RORgamma(t) in the generation of fetal lymphoid tissue inducer cells. Nat Immunol 5, 64-73). As shown here, RORγt is also expressed in populations of intestinal lamina propria T lymphocytes, most of which constitutively produce IL-17. Furthermore, these cells are absent in RORγt-deficient mice. In addition, both cytokine-directed in vitro differentiation of Th17 cells and in vivo Th17-mediated inflammatory disease require induction of RORγt. The results shown here demonstrate that RORγt is the transcription factor that directs the differentiation of inflammatory Th17 cells.

Using heterozygous mice in which a green fluorescent protein (GFP) reporter is under control of the Rorγt gene (Rorc(γt)^{+/gfp} mice), the inventors of the present application found that, in adult animals, RORγt is expressed in a third type of cells, namely the cryptopatch (CP) cells, which were found in ILFs and in the sub-epithelial dome of PPs, but not within the intestinal epithelium in mLNs or in periaortic LNs. CPs contained significant numbers of CD11c⁺ cells and were predominantly found in the small intestine. In contrast, ILFs consisted mainly of B cells, small numbers of αβ T cells and an activated VCAM-1⁺ stroma, and were predominantly found in the colon. Intestinal Rorγt⁺ cells expressed IL-7Rα and c-kit, and IL-7R⁺α cells were likewise positive for RORγt. Intestinal RORγt cells expressed both cKit and IL-7Rα and all lin⁻cKit⁺IL-7Rα+cells were likewise positive for RORγt. Furthermore, a subpopulation of Rorγt⁺ T cells was identified in the small intestine (but not the large intestine) and the colon of Rorc(γt)^{+/gfp} mice that produced IL-17.

Accordingly, the present invention provides the first demonstration of a molecule (RORγt) required for development of cryptopatches and of ILFs. Previous studies on cryptopatches proposed that they are precursors for intestinal T cells thought to develop independently of the thymus. The inventors' fate mapping studies shown herein clearly demonstrate that the RORγt-expressing cells in adult intestine are not precursors for lymphocytes or other differentiated hematopoietic cells, but are instead inducers of intestinal lymphoid tissues. Additionally, they showed that RORγt is required for the appearance of these inducer cells, and in its absence there is no organized lymphoid tissue in the gut. Because exposure to bacterial flora dictates the number and size of intestinal cryptopatches and of ILFs, the inventors propose that the RORγt-dependent intestinal inducer cells respond to external cues to initiate formation of inflammatory foci, the tertiary lymphoid tissues often found at sites of autoimmune disease.

While the developmental origin of intestinal intraepithelial T lymphocytes remains controversial, the inventors of the present application show here that intestinal αβ T cells are derived from precursors that express RORγt, an orphan nuclear hormone receptor detected only in immature CD4⁺CD8⁺ thymocytes (double positive or DP thymocytes), fetal lymphoid tissue inducer (LTi) cells, and adult intestinal cryptopatch (CP) cells. Using fate mapping, the inventors found that all intestinal αβ T cells are progeny of CD4+CD8+ thymocytes, but no intestinal T cells are derived from CP cells, which instead have a role similar to that of LTi cells in lymphoid tissue development in the adult gut.

The inventors of the present application have also demonstrated that the orphan nuclear receptor RORγt is the key transcription factor that orchestrates the differentiation of this effector cell lineage. RORγt induces transcription of the genes encoding IL-17 and the related cytokine IL-17F in naïve CD4⁺ T helper cells and is required for their expression in response to IL-6 and TGF-β. Th17 cells are constitutively present throughout the intestinal lamina propria, express RORγt, and are absent in mice deficient for RORγt or IL-6. Mice with RORγt-deficient T cells have attenuated autoimmune disease and lack tissue-infiltrating Th17 cells. Together, the studies presented in the present application suggest that RORγt is a key regulator of immune homeostasis and highlight its potential as a therapeutic target in inflammatory diseases.

It is with respect to these findings that the present invention is directed.

### Use of Antibodies Against RORγt Protein for Diagnostic or Therapeutic Purposes or for Screening for Novel Modulators of RORγt

One aspect of the invention provides a method of using an antibody against the RORγt gene product, e.g.protein (or peptides derived therefrom) or nucleic acids encoding RORγt, to diagnose a subject having or predisposed to having, a disease characterized by high levels of RORγt, such as inflammatory diseases, autoimmune diseases or individuals suffering from food allergies. Elevated levels of RORγt may be found in patients suffering from diseases such as arthritis, diabetes, multiple sclerosis, uveitis, rheumatoid arthritis, psoriasis, asthma, bronchitis, allergic rhinitis, chronic obstructive pulmonary disease, atherosclerosis, H. pylori infections and ulcers resulting from such infection, and inflammatory bowel diseases. Thus, in another aspect of the invention, one may look for a decrease in expression of the RORγt gene after appropriate therapy for these conditions. On the other hand, enhanced expression levels of the RORγt gene or gene product may be desirous when one is delivering a vaccine to an individual which should then lead to enhanced expression of the RORγt gene. Enhanced expression of the RORγt gene may then lead to induction of, or an increase in expression of certain cytokines that may play a role in enhanced immune responsiveness.

The diagnostic method of the invention provides contacting a biological sample such as a biopsy sample, tissue, or cell isolated from a subject with an antibody which binds RORγt. The antibody is allowed to bind to the RORγt antigen to form an antibody-antigen complex. The RORγt antigen, as used herein, includes the RORγt protein or peptides isolated therefrom. The conditions and time required to form the antibody-antigen complex may vary and are dependent on the biological sample being tested and the method of detection being used. Once non-specific interactions are removed by, for example, washing the sample, the antibody-antigen complex is detected using any immunoassay used to detect and/or quantitate antigens [see, for example, Harlow and Lane, Antibodies: A Laboratory Manual, Cold Spring Harbor Laboratory, New York (1988) 555-612]. Such well-known immunoassays include antibody capture assays, antigen capture assays, and two-antibody sandwich assays. In an antibody capture assay, the antigen is attached to solid support, and labeled antibody is allowed to bind. After washing, the assay is quantitated by measuring the amount of antibody retained on the solid support. In an antigen capture assay, the antibody is attached to a solid support, and labeled antigen is allowed to bind. The unbound proteins are removed by washing, and the assay is quantitated by measuring the amount of antigen that is bound. In a two-antibody sandwich assay, one antibody is bound to a solid support, and the antigen is allowed to bind to this first antibody. The assay is quantitated by measuring the amount of a labeled second antibody that binds to the antigen.

These immunoassays typically rely on labeled antigens, antibodies, or secondary reagents for detection. These proteins may be labeled with radioactive compounds, enzymes, biotin, or fluorochromes. Of these, radioactive labeling may be used for almost all types of assays. Enzyme-conjugated labels are particularly useful when radioactivity must be avoided or when quick results are needed. Biotin-coupled reagents usually are detected with labeled streptavidin. Streptavidin binds tightly and quickly to biotin and may be labeled with radioisotopes or enzymes. Fluorochromes, although requiring expensive equipment for their use, provide a very sensitive method of detection. Those of ordinary skill in the art will know of other suitable labels which may be employed in accordance with the present invention. The binding of these labels to antibodies or fragments thereof may be accomplished using standard techniques such as those described by Kennedy, et al. [(1976) Clin. Chim. Acta 70:1-31], and Schurs, et al. [(1977) Clin. Chim Acta 81:1-40].

In accordance with the diagnostic method of the invention, the presence or absence of the antibody-antigen complex is correlated with the presence or absence in the biological sample of the RORγt gene product. A biological sample containing elevated levels of the RORγt gene product is indicative of an inflammatory disease or an autoimmune disease or a food allergy. Examples of such diseases have been noted above. Accordingly, the diagnostic methods of the invention may be used as part of a routine screen in subjects suspected of having such diseases or for subjects who may be predisposed to having such diseases. Moreover, the diagnostic method of the invention may be used alone or in combination with other well-known diagnostic methods to confirm such diseases.

The diagnostic method of the invention further provides that an antibody of the invention may be used to monitor the levels of RORγt antigen in patient samples at various intervals of drug treatment to identify whether and to which degree the drug treatment is effective in restoring health. Furthermore, RORγt antigen levels may be monitored using an antibody of the invention in studies evaluating efficacy of drug candidates in model systems and in clinical trials. For example, using an antibody of this invention, RORγt antigen levels may be monitored in biological samples of individuals treated with known or unknown therapeutic agents. This may be accomplished with cell lines in vitro or in model systems and clinical trials, depending disease being investigated. Increased total levels of RORγt antigen in biological samples during or immediately after treatment with a drug candidate indicates that the drug candidate may actually exacerbate the disease. No change in total levels of RORγt antigen indicates that the drug candidate is ineffective in treating the disease. A lowering in total levels of RORγt antigen indicates that the drug candidate is effective in treating the disease. This may provide valuable information at all stages of pre-clinical drug development, clinical drug trials as well as subsequent monitoring of patients undergoing drug treatment. On the other hand, in situations where enhanced immunity is desired; i.e., where an individual is being vaccinated against a pathogen or tumor, treating such individual with an agent that increases expression of RORγt is desired. Such agonist or enhancer of RORγt may be delivered concomitantly with the vaccine or delivered independently of the vaccine.

The antibodies specific for RORγt may also be used to screen for small molecules that modulate the expression or activity/function of RORγt. A cell containing RORγt may be used for primary screening. Alternatively, any cell may be transfected with a vector containing the RORγt gene, and these cells may then be used to screen for candidate compounds that modulate RORγt expression or function. Such cells may include AKR cells, Phoenix cells, 293 cells, or any primary cell or cell line that has been transduced with the RORγt gene. After exposure to the cell in the presence or absence of the compound, the cells may be lysed and the proteins may be analyzed by any protocol known to those skilled in the art, and the antibodies described herein may be used to look for an increase or decrease in protein expression in untreated or compound treated cells. Some antibodies are commercially available (See R & D Systems, catalog number H6437), or they may be prepared using standard methods known to those skilled in the art (See the Example section). Standard procedures such as enzyme-linked immunosorbent assay (ELISA) or a Western blot may be used to monitor expression of the RORγt gene product (eg. protein) when screening for novel modulators. In one embodiment, a primary screen may involve the following: one may incubate cells that express the RORγt gene in the absence or presence of a candidate compound and would look for an increase or decrease of the RORγt protein in cells containing the gene after treatment with a candidate compound. A difference in the expression levels (an increase or decrease) of the RORγt protein after incubation with a candidate compound is an indication that the candidate compound acts as a modulator of RORγt. Subsequent or secondary screening may involve studying the effect of the candidate compound identified in the first or primary screen with a cell that is known to express any one of the proinflammatory cytokines, chemokines or their respective receptors, as described herein. A candidate compound that is shown to provide the expected results (such as a decrease in the expression or production of proinflammatory cytokines or chemokines) may then be tested in acceptable animal models, such as those described herein, for inflammatory diseases or autoimmune diseases.

### Detection of RORγt Nucleic Acid Molecules

In another particular embodiment, the invention involves methods to assess quantitative and qualitative aspects of RORγt gene or gene expression. In one example, the increased expression of RORγt gene or gene product indicates a predisposition for the development of an inflammatory disease or an autoimmune disease or a food allergy. Alternatively, enhanced expression levels of the RORγt gene or gene product may be desirous when one is delivering a vaccine to an individual which should then lead to enhanced expression of the RORγt gene. Techniques well known in the art, e.g., quantitative or semi-quantitative RT PCR or Northern blot, can be used to measure expression levels of the RORγt gene. Methods that describe both qualitative and quantitative aspects of RORγt gene or gene product expression are described in detail in the examples infra. The measurement of RORγt gene expression levels may include measuring naturally occurring RORγt transcripts and variants thereof as well as non-naturally occurring variants thereof. The diagnosis and/or prognosis of an inflammatory disease, an autoimmune disorder, or a food allergy in a subject, however, is preferably directed to detecting increased levels of a naturally occurring RORγt gene product or variant thereof. Thus, the invention relates to methods of diagnosing and/or predicting an inflammatory disease or an autoimmune disease or a food allergy in a subject by measuring the expression of an RORγt gene or gene product in a subject. For example, the increased level of mRNA encoded by an RORγt gene (e.g., SEQ ID NO: 1), as compared to a normal sample or a predetermined normal standard would indicate the presence of an inflammatory disease or an autoimmune disease or a food allergy in said subject or the increased risk of developing an inflammatory disease or an autoimmune disease or a food allergy in said subject.

In another aspect of the invention, the increased level of mRNA encoded for by a RORγt gene (e.g., SEQ ID NO: 1, human DNA having accession number NM_001001523, or SEQ ID NO: 3, mouse DNA having accession number AF163668), or other related gene products (e.g., SEQ ID NO: 2, human protein, or SEQ ID NO: 4, mouse protein), as compared to that of a normal sample or a predetermined normal standard would indicate the stage of disease in said subject or the likelihood of a poor prognosis in said subject.

In another example, RNA from a cell type or tissue known, or suspected, to express a RORγt gene, may be isolated and tested utilizing hybridization or PCR techniques as described above. The isolated cells can be derived from cell culture or from a patient. The analysis of cells taken from culture may be a necessary step in the assessment of cells to be used as part of a cell-based gene therapy technique or, alternatively, to test the effect of compounds on the expression of the RORγt gene. Such analyses may reveal both quantitative and qualitative aspects of the expression pattern of the RORγt gene, including activation or suppression of RORγt gene expression and the presence of alternatively spliced RORγt gene transcripts.

In one embodiment of such a detection scheme, a cDNA molecule is synthesized from an RNA molecule of interest by reverse transcription. All or part of the resulting cDNA is then used as the template for a nucleic acid amplification reaction, such as a PCR or the like. The nucleic acid reagents used as synthesis initiation reagents (e.g., primers) in the reverse transcription and nucleic acid amplification steps of this method are chosen from among RORγt gene nucleic acid reagents. The preferred lengths of such nucleic acid reagents are at least 9-30 nucleotides.

For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

RT-PCR techniques can be utilized to detect differences in RORγt gene transcript size that may be due to normal or abnormal alternative splicing. Additionally, such techniques can be performed using standard techniques to detect quantitative differences between levels of RORγt gene transcripts detected in normal individuals relative to those individuals having an inflammatory disease, an autoimmune disease or a food allergy or exhibiting a predisposition towards these conditions.

In the case where detection of particular alternatively spliced species is desired, appropriate primers and/or hybridization probes can be used, such that, in the absence of such a sequence, for example, no amplification would occur.

As an alternative to amplification techniques, standard Northern analyses can be performed if a sufficient quantity of the appropriate cells or tissue can be obtained. The preferred length of a probe used in a Northern analysis is 9-50 nucleotides. Utilizing such techniques, quantitative as well as size related differences between RORγt transcripts can also be detected.

Additionally, it is possible to perform such RORγt gene expression assays in situ, i.e., directly upon tissue sections (fixed and/or frozen) of patient tissue obtained from biopsies or resections, such that no nucleic acid purification is necessary. Nucleic acid reagents such as those described herein may be used as probes and/or primers for such in situ procedures (see, e.g., Nuovo, G. J., 1992, PCR In Situ Hybridization: Protocols And Applications, Raven Press, NY).

Mutations or polymorphisms within a RORγt gene can be detected by utilizing a number of techniques. Nucleic acid from any nucleated cell (e.g., genomic DNA) can be used as the starting point for such assay techniques, and may be isolated according to standard nucleic acid preparation procedures that are well known to those of skill in the art. For the detection of RORγt transcripts or RORγt gene products, any cell type or tissue in which the RORγt gene is expressed may be utilized.

Genomic DNA may be used in hybridization or amplification assays of biological samples to detect abnormalities involving RORγt gene structure, including point mutations, insertions, deletions and chromosomal rearrangements. Such assays may include, but are not limited to, direct sequencing (Wong, C. et al., 1987, Nature 330:384), single stranded conformational polymorphism analyses (SSCP; Orita, M. et al., 1989, Proc. Natl. Acad. Sci. USA 86:2766), heteroduplex analysis (Keen, T. J. et al., 1991, Genomics 11:199; Perry, D. J. & Carrell, R. W., 1992), denaturing gradient gel electrophoresis (DGGE; Myers, R. M. et al., 1985, Nucl. Acids Res. 13:3131), chemical mismatch cleavage (Cotton, R. G. et al., 1988, Proc. Natl. Acad. Sci. USA 85:4397) and oligonucleotide hybridization (Wallace, R. B. et al., 1981, Nucl. Acids Res. 9:879; Lipshutz, R. J. et al., 1995, Biotechniques 19:442).

Diagnostic methods for the detection of RORγt gene nucleic acid molecules, in patient samples or other appropriate cell sources, may involve the amplification of specific gene sequences, e.g., by PCR (See Mullis, K. B., 1987, U.S. Pat. No. 4,683,202), followed by the analysis of the amplified molecules using techniques well known to those of skill in the art, such as, for example, those listed above. Utilizing analysis techniques such as these, the amplified sequences can be compared to those that would be expected if the nucleic acid being amplified contained only normal copies of a RORγt gene in order to determine whether a RORγt gene mutation exists.

Microarrays may also be used for determining RORγt gene expression levels or other genes that are modulated by RORγt and may be prepared by methods known in the art, or they may be custom made by companies, e.g., Affymetrix (Santa Clara, Calif.) (see www.affymetrix.com). Numerous articles describe the different microarray technologies, (e.g., Shena, et al., Tibtech, (1998), 16: 301; Duggan, et al., Nat. Genet., (1999), 21:10; Bowtell, et al., Nat. Genet., (1999), 21:25; Hughes, et al., Nat. Biotechn., (2001), 19:342). While many of the microarrays utilize nucleic acids and relevant probes for the analysis of gene expression profiles, protein arrays, in particular, antibody arrays or glycosylation arrays also hold promise for studies related to protein or glycoprotein expression from biological samples (see for example, RayBiotech, Inc. at www.raybiotech.com/product.htm, Panomics at www.panomics.com, Clontech Laboratories, inc. at www.clontech.com, Procognia in Maidenhead, UK and Qiagen at www.qiagen.com.

### Therapeutic and Prophylactic Compositions and Their Use

Candidates for therapy with the agents identified by the methods described herein are patients either suffering from an inflammatory disease, an autoimmune disorder or a food allergy or are prone to development of such disorders. In this situation, the agents would be modulators of RORγt, preferably inhibitors or antagonists of RORγt. Furthermore, if the “stem cell” hypothesis for cancers is correct, then treatment of these cancers with a combination of an RORγt inhibitor (to block at the progenitor double positive stage) with chemotherapy to eliminate differentiated tumor may be effective. In addition, patients in need of being vaccinated against certain pathogenic organisms, e.g. bacteria, viruses, fungi, parasites or tumors may be in need of treatment with an agent that enhances the expression of RORγt, or with an agonist that enhances the expression and/or activity of RORγt.

The invention provides methods of treatment comprising administering to a subject an effective amount of an agent that modulates the expression and/or activity of RORγt. A “modulator of RORγt” is defined as an agent that acts as an agonist or stimulator that enhances expression and/or activity of RORγt or an antagonist that decreases expression and/or activity of RORγt. The agent may be identified as a compound, such as a small organic molecule that acts to antagonize expression of RORγt, or it may be a protein or polypeptide, a nucleic acid molecule such as an antisense molecule or a small interfering nucleic acid molecule, such as a siRNA or a shRNA molecule that prevents expression or function of RORγt. It may be an antagonistic antibody that decreases expression of RORγt, for treatment of diseases such as inflammatory conditions, autoimmune diseases or food allergies.

One approach for use of an antagonistic anti-RORγt antibody is via insertion of the gene encoding the antibody into a cell whereby the intracellular expression of the antibody gene allows for modulation of the function of the protein for which the antibody is specific. Accordingly, this invention provides for methods and compositions for modulating RORγt expression and/or function in a cell involving intracellular expression of such an antagonistic antibody that binds to RORγt within the cell, thereby altering the function of this protein. The invention is particularly applicable to inhibiting the expression of RORγt in an immune cell, such as a T lymphocyte, thus inhibiting potential effects of RORγt on induction of pro-inflammatory cytokines or chemokines. Such an approach may be used in treating inflammatory or autoimmune diseases, caused in part by the presence of RORγt in immune cells.

To express an antibody within a cell, a nucleic acid molecule encoding the antibody, such as a recombinant expression vector encoding the antibody, is introduced into the cell. Preferably, the antibody used to modulate protein expression or function is a single chain Fv (scFv) fragment, although whole antibodies, or antigen binding fragments thereof (e.g., Fab fragments) may also be useful.

In a particularly preferred embodiment of the invention, an antibody is expressed intracellularly in a mammalian immune cell to inhibit the effects of RORγt on induction of proinflammatory cytokines or chemokines. The target cells of interest may be selected from any immune cell in which RORγt plays a role in enhancement of expression of such proinflammatory molecules, such as T cells, including CD4+ and CD8+ T cells. A nucleic acid molecule encoding the antibody can be introduced in vivo into cells of interest, by, for example, use of a recombinant viral vector or other vector system suitable for delivery of genes to cells in vivo.

An isolated nucleic acid molecule encoding an antibody can be prepared according to standard molecular biology methods using nucleic acid sequences obtained from antibody genes. Isolated nucleic acid molecules encoding antibody chains (or relevant antigen binding portions thereof, such as V_{H} or V_{L} regions), specific for many different particular proteins have been described, and/or are available, in the art. Additionally, such nucleic acids can be isolated by standard techniques, for example, from a hybridoma that expresses a monoclonal antibody specific for a protein of interest, such as RORγt, or by screening an immunoglobulin expression library (e.g., an immunoglobulin phage display library) with the protein of interest. Antibodies specific for RORγt are commercially available (see R&D Systems, catalogue No. H6437) although one may contemplate preparing other monoclonal or polyclonal antibodies by standard procedures known to those skilled in the art.

Alternatively, it may be desirous to treat with an agent that increases expression of RORγt, such as an agonist that can be used with a vaccine candidate for various pathogenic organisms or with a tumor vaccine. The agent that acts as an agonist may be identified as a compound, such as a small organic molecule that acts to stimulate expression of RORγt, or it may be a protein or polypeptide, or a nucleic acid molecule. It is envisioned that agonists may be developed that act directly on expression and/or activity of the RORγt protein. These agents may be used alone or in combination with other standard treatment regimens or strategies that are commonly used for the specific disease being treated. In a preferred aspect, the compound is substantially purified (e.g., substantially free from substances that limit its effect or produce undesired side-effects). The subject is preferably an animal, including but not limited to animals such as monkeys, cows, pigs, horses, chickens, cats, dogs, etc., and is preferably a mammal, and most preferably human. In one specific embodiment, a non-human mammal is the subject. In another specific embodiment, a human mammal is the subject. Accordingly, the agents identified by the methods described herein may be formulated as pharmaceutical compositions to be used for prophylaxis or therapeutic use to treat these patients.

Various delivery systems are known and can be used to administer a compound of the invention, e.g., encapsulation in liposomes, microparticles, or microcapsules. Methods of introduction can be enteral or parenteral and include but are not limited to intradermal, intramuscular, intraperitoneal, intravenous, subcutaneous, intranasal, epidural, topical and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. In a specific embodiment, it may be desirable to administer the pharmaceutical compositions of the invention locally to the area in need of treatment.

Such compositions comprise a therapeutically effective amount of an agent, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E.W. Martin. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

In a specific embodiment, it may be desirable to administer the pharmaceutical compositions of the invention locally to the area in need of treatment; this may be achieved, for example, and not by way of limitation, by local infusion during surgery, by topical application, by injection, by means of a catheter, or by means of an implant, said implant being of a porous, non-porous, or gelatinous material, including membranes, such as sialastic membranes, or fibers or co-polymers such as Elvax (see Ruan et al , 1992, Proc Natl Acad Sci USA, 89:10872-10876). In one embodiment, administration can be by direct injection by aerosol inhaler.

In another embodiment, the compound can be delivered in a vesicle, in particular a liposome (see Langer (1990) Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton (1987) CRC Crit. Ref. Biomed. Eng. 14:201; Buchwald et al. (1980) Surgery 88:507; Saudek et al. (1989) N. Engl. J. Med. 321:574). In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, J. (1983) Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target, i.e., the airways, thus requiring only a fraction of the systemic dose (see, e.g., Goodson, in Medical Applications of Controlled Release (1984) supra, vol. 2, pp. 115-138). Other suitable controlled release systems are discussed in the review by Langer (1990) Science 249:1527-1533.

### Effective Doses

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). Candidate agonists and antagonists would be tested in wild type and RORγt knockout (ko) mice, to show lack of an effect in the ko mice. However, candidate drugs will also tested in other animals as well (rats, dogs). Generally, the target would first be to human RORγt, and then would be tested for cross-species effects in mouse (and other species). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects can be used, care should be taken to design a delivery system that targets such compounds to the site of affected tissue in order to minimize potential damage to unaffected cells and, thereby, reduce side effects.

The data obtained from cell culture assays and animal studies can be used in formulating a dose range for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to optimize efficacious doses for administration to humans. Plasma levels can be measured by any technique known in the art, for example, by high performance liquid chromatography.

In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. Normal dose ranges used for particular therapeutic agents employed for specific diseases can be found in the Physicians' Desk Reference, 54^{th} Edition (2000).

Treatments may also be achieved by administering DNA encoding the agents that increase or decrease the expression of the RORγt gene described above in an expressible genetic construction. DNA encoding the agent, e.g. in the event said agent is a protein or polypeptide, may be administered to the patient using techniques known in the art for delivering DNA to the cells. For example, retroviral vectors, electroporation or liposomes may be used to deliver DNA.

The invention includes use of any modifications or equivalents of the above agents which do not exhibit a significantly reduced or increased activity as related to RORγt gene expression. For example, modifications in which amino acid content or sequence is altered without substantially adversely affecting activity are included. The statements of effect and use contained herein are therefore to be construed accordingly, with such uses and effects employing modified or equivalent gene products being part of the invention.

The present agents that enhance expression of RORγt or the RORγt genes or gene products themselves can be used as the sole active agents, or can be used in combination with other active ingredients.

### Candidate Compounds and Agents

As used herein, the term “candidate compound” or “test compound” refers to any compound or molecule that is to be tested. As used herein, the terms, which are used interchangeably, refer to biological or chemical compounds such as simple or complex organic or inorganic molecules, peptides, proteins, peptidomimetics, peptide mimics, antibodies, nucleic acids (DNA or RNA), oligonucleotides, polynucleotides, antisense molecules, small interfering nucleic acid molecules, including siRNA or shRNA, carbohydrates, lipoproteins, lipids, small molecules and other drugs. A vast array of compounds can be synthesized, for example oligomers, such as oligopeptides and oligonucleotides, and synthetic organic compounds based on various core structures, and these are also included in the terms noted above. In addition, various natural sources can provide compounds for screening, such as plant or animal extracts, and the like. Compounds can be tested singly or in combination with one another. Agents or candidate compounds can be randomly selected or rationally selected or designed. As used herein, an agent or candidate compound is said to be “randomly selected” when the agent is chosen randomly without considering the specific interaction between the agent and the target compound or site. As used herein, an agent is said to be “rationally selected or designed”, when the agent is chosen on a nonrandom basis which takes into account the specific interaction between the agent and the target site and/or the conformation in connection with the agent's action. Moreover, the agent may be selected by its effect on the gene expression profile obtained from screening in vitro or in vivo. Furthermore, candidate compounds can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145; U.S. Pat. No. 5,738,996; and U.S. Pat. No. 5,807,683).

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented, e.g., presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310).

If the screening for compounds that modulate the expression, activity or function of RORγt, is done with a library of compounds, it may be necessary to perform additional tests to positively identify a compound that satisfies all required conditions of the screening process. There are multiple ways to determine the identity of the compound. One process involves mass spectrometry, for which various methods are available and known to the skilled artisan (see for instance neogenesis.com). In addition, a secondary screen may include assessing the effect of a candidate compound on the expression or activity of a proinflammatory cytokine or cytokine receptor or a proinflammatory chemokine or chemokine receptor using standard procedures known in the art.

### Screening/Testing for Modulators of RORγt

Any screening technique known in the art can be used to screen for active or positive candidate compounds that modulate RORγt expression or activity/function. The present invention contemplates screens for small molecule modulators, as well as screens for natural proteins or peptides that bind to and modulate RORγt expression and/or activity or function. For example, natural products or peptide libraries can be screened using assays of the invention for molecules that have the ability to alter the proinflammatory cytokine or chemokine profile from immune cells, eg. to inhibit the expression, production and/or release of pro-inflammatory cytokines or chemokines or to enhance the expression, production and/or release of pro-inflammatory cytokines or chemokines from immune cells.

Identification and screening of a molecule is further facilitated by determining structural features of the protein, e.g., using X-ray crystallography, neutron diffraction, nuclear magnetic resonance spectrometry, and other techniques for structure determination. These techniques provide for the rational design or identification of proteins, or peptide fragments that have a modulatory effect on RORγt expression or activity or function.

Another approach uses recombinant bacteriophage to produce large libraries. Using the “phage method” [Scott and Smith, 1990, Science 249:386-390 (1990); Cwirla, et al., Proc. Natl. Acad. Sci., 87:6378-6382 (1990); Devlin et al., Science, 249:404-406 (1990)], very large libraries can be constructed (10⁶-10⁸ chemical entities). A second approach uses primarily chemical methods, of which the Geysen method [Geysen et al., Molecular Immunology 23:709-715 (1986); Geysen et al. J. Immunologic Method 102:259-274 (1987)] and the method of Fodor et al. [Science 251:767-773 (1991)] are examples. Furka et al. [14th International Congress of Biochemistry, Volume 5, Abstract FR:013 (1988); Furka, Int. J. Peptide Protein Res. 37:487-493 (1991)], Houghton [U.S. Pat. No. 4,631,211, issued December 1986] and Rutter et al. [U.S. Pat. No. 5,010,175, issued Apr. 23, 1991] describe methods to produce a mixture of peptides that can be tested as activators or inhibitors.

Screening phage-displayed random peptide libraries offers a rich source of molecular diversity and represents a powerful means of identifying peptide ligands that bind a receptor molecule of interest (Cwirla, et al., Proc. Natl. Acad. Sci., 87:6378-6382 (1990); Devlin et al., Science, 249:404-406 (1990)). Phage expressing binding peptides are selected by affinity purification with the target of interest. This sytem allows a large number of phage to be screened at one time. Since each infectious phage encodes the random sequence expressed on its surface, a particular phage, when recovered from an affinity matrix, can be amplified by another round of infection. Thus, selector molecules immobilized on a solid support can be used to select peptides that bind to them. This procedure reveals a number of peptides that bind to the selector and that often display a common consensus amino acid sequence. Biological amplification of selected library members and sequencing allows the determination of the primary structure of the peptide(s).

Peptides are expressed on the tip of the filamentous phage M13, as a fusion protein with the phage surface protein pilus (at the N-terminus). Typically, a filamentous phage carries on its surface 3 to 5 copies of pili and therefore of the peptide. In such a system, no structural constraints are imposed on the N-terminus; the peptide is therefore free to adopt many different conformations, allowing for a large diversity. However, biases in the distribution of peptides in the library may be caused by biological selection against certain of the peptides, which could reduce the diversity of peptides contained in the library. In practice, this does not appear to be a significant problem. When randomly selected peptides expressed at the N-terminus of pili were analyzed (Cwirla, et al., Proc. Natl. Acad. Sci., 87:6378-6382 (1990)), most amino acids appeared at each position of the variable peptide, indicating that no severe discrimination against particular amino acids had occurred. Selection against particular combinations of amino acids would however not have been detected in this analysis.

In another aspect, synthetic libraries [Needels et al., Proc. Natl. Acad. Sci. USA 90:10700-4 (1993); Ohlmeyer et al., Proc. Natl. Acad. Sci. USA 90:10922-10926 (1993); Lam et al., International Patent Publication No. WO 92/00252; Kocis et al., International Patent Publication No. WO 9428028, each of which is incorporated herein by reference in its entirety], and the like can be used to screen for novel peptides or mimics thereof or fragments thereof according to the present invention.

Alternatively, the effect of a candidate compound may be tested on immune cells, such as T cells or macrophages obtained from tissues, or blood, or on a T cell or macrophage cell line, such as the RAW264.7 cell line or the U937 cell line. For example, one may assess the effects of the candidate compound on the cytokine profile (on the gene or protein) in these cells, or on release of one or more cytokines from these cells. A positive candidate would alter the cytokine profile such that the pro-inflammatory cytokines (such as but not limited to IL-1, IL-6, IL-12, TNF alpha) would be reduced.

The methods used to measure the effect of the candidate compound on T cells or macrophages, more particularly, on the cytokine expression profile, may include standard procedures known to those skilled in the art. For example, the level of expression of a gene or gene product (protein) may be determined by a method selected from, but not limited to, cDNA microarray, reverse transcription-polymerase chain reaction (RT-PCR), real time PCR and proteomics analysis. Other means such as electrophoretic gel analysis, enzyme immunoassays (ELISA assays), Western blots, dotblot analysis, Northern blot analysis and in situ hybridization may also be contemplated for use, although it is to be understood that the former assays that are noted (eg. micrarrays, RT-PCR, real time PCR and proteomics analysis) provide a more sensitive, quantitative and reliable measurement of genes or gene products that are modulated by a candidate compound. Sequences of the genes or cDNA from which probes are made (if needed) for analysis may be obtained, e.g., from GenBank.

### Use of RORγt Modulators for Treatment of Immune Mediated Diseases

As noted above, a compound that modulates the expression of RORγt may be used to treat immune mediated diseases associated with the presence of inflammatory cells and the inflammatory mediators produced by these cells. In a preferred embodiment, the agent for treating an immune mediated disease or condition, whereby the immune mediated disease is an inflammatory condition would be an antagonist or inhibitor of RORγt expression. The treatment with such an antagonist may diminish the tissue damage associated with the presence of the inflammatory cells and mediators. The diseases for which treatment with a modulator of RORγt expression may be effective are summarized below. An antagonist of RORγt for treating such diseases may be an antibody to RORγt, such as one that may be commercially available (see R&D Systems catalog number H6437). Alternatively, such an antibody may be made by standard techniques known to those skilled in the art. Furthermore, an antagonist to RORγt may be an antisense molecule, or a siRNA or shRNA molecule. Such shRNA molecule has been prepared and tested, and has been shown to inhibit the expression of RORγt, using the sequence shown in SEQ ID NOS: 9 and 10. Others may be prepared using the sequence of RORγt as known (See SEQ ID NO: 1 or 3). In addition, given the known sequence of RORγt, other small interfering nucleic acid molecules may be prepared, including siRNA or shRNA molecules using techniques known to those skilled in the art.

### Antisense Molecules

Anti-sense nucleic acid molecules which are complementary to nucleic acid sequences contained within an RORγt gene as shown in SEQ ID NO: 1), can be used to treat an inflammatory condition, in which the expression level of a RORγt gene is elevated in immune cells as compared to that of normal cells or a predetermined standard. Thus, in one embodiment of the invention a method for treating an inflammatory condition is provided whereby a patient suffering from such condition is treated with an effective amount of a RORγt gene anti-sense nucleic acid molecule.

Antisense approaches involve the design of oligonucleotides (either DNA or RNA) that are complementary to RORγt gene mRNA. The antisense oligonucleotides bind to RORγt gene mRNA transcripts and thereby prevent translation. Absolute complementarity, although preferred, is not required. “Complementary” is understood in its recognized meaning as identifying a nucleotide in one sequence that hybridizes (anneals) to a nucleotide in another sequence according to the rule A→T, U and C→G (and vice versa) and thus “matches” its partner for purposes of this definition. Enzymatic transcription has measurable and well known error rates (depending on the specific enzyme used), thus within the limits of transcriptional accuracy using the modes described herein, in that a skilled practitioner would understand that fidelity of enzymatic complementary strand synthesis is not absolute and that the amplicon need not be completely matched in every nucleotide to the target or template RNA. Thus, a sequence “complementary” to a portion of an RNA, as referred to herein, means a sequence having sufficient complementarity to be able to hybridize with the non-poly A portion of the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches it may comprise and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, are considered preferred for antisense applications because, in general, they efficiently inhibit translation. However, sequences complementary to the 3′ untranslated sequences of mRNAs have also been shown to be effective at inhibiting translation of mRNAs as well. (See generally, Wagner, R., 1994, Nature 372:333). Thus, oligonucleotides complementary to the 5′-non-translated region, the 3′-non-translated region, or any other suitable region of the transcript (e.g., part of a coding region) could be used in an antisense approach to inhibit translation of endogenous RORγt gene mRNA.

Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′-, or coding region of a gene mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared to those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. USA 86:6553; Lemaitre et al., 1987, Proc. Natl. Acad. Sci. USA 84:648; PCT Publication No. WO88/09810, published Dec. 15, 1988) or the blood-brain barrier (see, e.g., PCT Publication No. WO89/10134, published Apr. 25, 1988), hybridization-triggered cleavage agents. (see, e.g., Krol et al., 1988, BioTechniques 6:958) or intercalating agents. (see, e.g., Zon, 1988, Pharm. Res. 5:539). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5 -methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625). The oligonucleotide is a 2-0-methylribonucleotide (Inoue et al., 1987, Nucl. Acids Res. 15:6131), or a chimeric RNA-DNA analogue (Inoue et al., 1987, FEBS Lett. 215:327).

An RORγt gene antisense nucleic acid sequence can comprise the complement of any contiguous segment within the sequence of the RORγt gene of the invention (SEQ ID NO: 1).

In one embodiment of the present invention, a RORγt antisense nucleic acid sequence is about 50 bp in length. In certain specific embodiments, a RORγt antisense nucleic acid sequence comprises a sequence complementary to any contiguous 50 bp stretch of nucleotides of SEQ ID NO: 1

In another embodiment a RORγt antisense nucleic acid sequence is about 100 bp in length. In certain specific embodiments, a RORγt antisense nucleic acid sequence comprises a sequence complementary to any contiguous 100 bp stretch of nucleotides of SEQ ID NO: 1

In another embodiment a RORγt antisense nucleic acid sequence is about 200 bp in length. In a particular embodiment, a RORγt antisense nucleic acid sequence comprises a sequence complementary to any contiguous 200 bp stretch of nucleotides of SEQ ID NO: 1.

In another embodiment a RORγt antisense nucleic acid sequence is about 400 bp in length. In a particular embodiment, a RORγt antisense nucleic acid sequence comprises a sequence complementary to any contiguous 400 bp stretch of nucleotides of SEQ ID NO: 1.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448), etc.

While antisense nucleotides complementary to a RORγt coding region could be used, those complementary to the transcribed untranslated region may also be used.

Antisense molecules are delivered to cells that express the RORγt gene in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

It is often difficult, however, to achieve intracellular concentrations of the antisense sufficient to suppress translation of endogenous mRNAs. Therefore, a preferred approach utilizes a recombinant DNA construct in which the antisense oligonucleotide is placed under the control of a strong pol III or pol II promoter. The use of such a construct to transfect target cells in the patient results in the transcription of sufficient amounts of single stranded RNAs that form complementary base pairs with the endogenous RORγt gene transcripts and thereby prevent translation of the RORγt gene mRNA. For example, a vector can be introduced in vivo such that it can be taken up by a cell and direct the transcription of an antisense RNA. Such a vector may remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be effected by any promoter known in the art to act in mammalian, preferably human cells. Such promoters can be inducible or constitutive. Such promoters include but are not limited to: the SV40 early promoter region (Bernoist and Chambon, 1981, Nature 290:304), the promoter contained in the 3 long terminal repeat of Rous sarcoma virus (Yamamoto et al., 1980, Cell 22:787), the herpes thymidine kinase promoter (Wagner et al., 1981, Proc. Natl. Acad. Sci. USA 78:1441), the regulatory sequences of the metallothionein gene (Brinster et al., 1982, Nature 296:39), etc. Any type of plasmid, cosmid, YAC or viral vector can be used to prepare the recombinant DNA construct that can be introduced directly into the tissue site. Alternatively, viral vectors can be used which selectively infect the desired tissue.

An effective dose of an L antisense oligonucleotide to be administered during a treatment cycle ranges from about 0.01 to 0.1, 0.1 to 1, or 1 to 10 mg/kg/day. The dose of RORγt antisense oligonucleotide to be administered can be dependent on the mode of administration. For example, intravenous administration of a RORγt antisense oligonucleotide would likely result in a significantly higher systemic dose than a systemic dose resulting from a local implant containing a pharmaceutical composition comprising an RORγt antisense oligonucleotide. In one embodiment, a RORγt antisense oligonucleotide is administered subcutaneously at a dose of 0.01 to 10 mg/kg/day. In another embodiment, a RORγt antisense oligonucleotide is administered intravenously at a dose of 0.01 to 10 mg/kg/day. In yet another embodiment, a RORγt antisense oligonucleotide is administered locally at a dose of 0.01 to 10 mg/kg/day. It will be evident to one skilled in the art that local administrations may result in lower systemic or total body doses. For example, local administration methods such as intratumor administration, intraocular injection, or implantation, can produce locally high concentrations of RORγt antisense oligonucleotide, but represent a relatively low dose with respect to total body weight. Thus, in such cases, local administration of a RORγt antisense oligonucleotide is contemplated to result in a total body dose of about 0.01 to 5 mg/kg/day.

In another embodiment, a particularly high dose of a RORγt antisense oligonucleotide, which ranges from about 10 to 50 mg/kg/day, is administered during a treatment cycle.

Moreover, the effective dose of a particular RORγt antisense oligonucleotide may depend on additional factors, including the type of disease, the disease state or stage of disease, the oligonucleotide's toxicity, the oligonucleotide's rate of uptake by cancer cells, as well as the weight, age, and health of the individual to whom the antisense oligonucleotide is to be administered. Because of the many factors present in vivo that may interfere with the action or biological activity of a RORγt antisense oligonucleotide, one of ordinary skill in the art can appreciate that an effective amount of an RORgt antisense oligonucleotide may vary for each individual.

In another embodiment, a RORγt antisense oligonucleotide is administered at a dose which results in circulating plasma concentrations of a RORγt antisense oligonucleotide that are at least 50 nM (nanomolar). As will be apparent to the skilled artisan, lower or higher plasma concentrations of an RORγt antisense oligonucleotide may be preferred depending on the mode of administration. For example, plasma concentrations of a RORγt antisense oligonucleotide of at least 50 nM can be appropriate in connection with, e.g., intravenous, subcutaneous, intramuscular, controlled release, and oral administration methods. In another example, relatively low circulating plasma levels of an RORγt antisense oligonucleotide can be desirable, however, when using local administration methods such as, for example, intratumor administration, intraocular administration, or implantation, which nevertheless can produce locally high, clinically effective concentrations of RORγt antisense oligonucleotide.

A high dose may also be achieved by several administrations per cycle. Alternatively, the high dose may be administered in a single bolus administration. A single administration of a high dose may result in circulating plasma levels of RORγt antisense oligonucleotide that are transiently much higher than 50 nM.

Additionally, the dose of a RORγt antisense oligonucleotide may vary according to the particular RORγt antisense oligonucleotide used. The dose employed is likely to reflect a balancing of considerations, among which are stability, localization, cellular uptake, and toxicity of the particular RORγt antisense oligonucleotide. For example, a particular chemically modified RORγt antisense oligonucleotide may exhibit greater resistance to degradation, or may exhibit higher affinity for the target nucleic acid, or may exhibit increased uptake by the cell or cell nucleus; all of which may permit the use of low doses. In yet another example, a particular chemically modified RORγt antisense oligonucleotide may exhibit lower toxicity than other antisense oligonucleotides, and therefore can be used at high doses. Thus, for a given RORγt antisense oligonucleotide, an appropriate dose to administer can be relatively high or low. The invention contemplates the continued assessment of optimal treatment schedules for particular species of RORγt antisense oligonucleotides. The daily dose can be administered in one or more treatments.

A “low dose” or “reduced dose” refers to a dose that is below the normally administered range, i.e., below the standard dose as suggested by the Physicians' Desk Reference, 54^{th} Edition (2000) or a similar reference. Such a dose can be sufficient to inhibit cell proliferation, or demonstrates ameliorative effects in a human, or demonstrates efficacy with fewer side effects as compared to standard cancer treatments. Normal dose ranges used for particular therapeutic agents and standard cancer treatments employed for specific diseases can be found in the Physicians' Desk Reference, 54^{th} Edition (2000) or in Cancer: Principles & Practice of Oncology, DeVita, Jr., Hellman, and Rosenberg (eds.) 2nd edition, Philadelphia, Pa.: J.B. Lippincott Co., 1985.

Reduced doses of an RORγt nucleic acid molecule, an RORγt polypeptide, an RORγt antagonist, and/or a combination therapeutic may demonstrate reduced toxicity, such that fewer side effects and toxicities are observed in connection with administering an RORγt antagonist and one or more cancer therapeutics for shorter duration and/or at lower doses when compared to other treatment protocols and dosage formulations, including the standard treatment protocols and dosage formulations as described in the Physicians' Desk Reference, 54^{th} Edition (2000) or in Cancer: Principles & Practice of Oncology, DeVita, Jr., Hellman, and Rosenberg (eds.) 2nd edition, Philadelphia, Pa.: J.B. Lippincott Co., 1985.

A “treatment cycle” or “cycle” refers to a period during which a single therapeutic or sequence of therapeutics is administered. In some instances, one treatment cycle may be desired, such as, for example, in the case where a significant therapeutic effect is obtained after one treatment cycle. The present invention contemplates at least one treatment cycle, generally preferably more than one treatment cycle.

Other factors to be considered in determining an effective dose of a RORγt antisense oligonucleotide include whether the oligonucleotide will be administered in combination with other therapeutics. In such cases, the relative toxicity of the other therapeutics may indicate the use of a RORγt antisense oligonucleotide at low doses. Alternatively, treatment with a high dose of RORγt antisense oligonucleotide can result in combination therapies with reduced doses of therapeutics. In a specific embodiment, treatment with a particularly high dose of RORγt antisense oligonucleotide can result in combination therapies with greatly reduced doses of cancer therapeutics. For example, treatment of a patient with 10, 20, 30, 40, or 50 mg/kg/day of a RORγt antisense oligonucleotide can further increase the sensitivity of a subject to cancer therapeutics. In such cases, the particularly high dose of RORgt antisense oligonucleotide is combined with, for example, a greatly shortened radiation therapy schedule. In another example, the particularly high dose of a RORγt antisense oligonucleotide produces significant enhancement of the potency of cancer therapeutic agents.

Additionally, the particularly high doses of RORγt antisense oligonucleotide may further shorten the period of administration of a therapeutically effective amount of RORγt antisense oligonucleotide and/or additional therapeutic, such that the length of a treatment cycle is much shorter than that of the standard treatment.

The invention contemplates other treatment regimens depending on the particular RORγt antisense oligonucleotide to be used, or depending on the particular mode of administration, or depending on whether an RORγt antisense oligonucleotide is administered as part of a combination therapy, e.g., in combination with a cancer therapeutic agent. The daily dose can be administered in one or more treatments.

siRNA Therapy

In general terms, RNA interference (RNAi) is the process whereby the introduction of double stranded RNA into a cell inhibits the expression of a gene corresponding to its own sequence. RNAi is usually described as a post-transcriptional gene-silencing (PTGS) mechanism in which dsRNA triggers degradation of homologous messenger RNA in the cytoplasm. The mediators of RNA interference are 21- and 23-nucleotide small interfering RNAs (siRNA) (Elbashir, S. M. et al., (2001), Genes Dev. 15, 188-200; Elbashir, S. M. et al. (2001), Nature 411: 494-498; Hutvagner, G. et al., (2001), Science 293:834-838). In a second step, siRNAs bind to a ribonuclease complex called RNA-induced silencing complex (RISC) that guides the small dsRNAs to its homologous mRNA target. Consequently, RISC cuts the mRNA approximately in the middle of the region paired with the antisense siRNA, after which the mRNA is further degraded. A ribonuclease III enzyme, dicer, is required for processing of long dsRNA into siRNA duplexes (Bernstein, E. et al. ((2001), Nature 409: 363-366).

### Mechanism of RNAi

The only RNA molecules normally found in the cytoplasm of a cell are molecules of single-stranded mRNA. If the cell finds molecules of double-stranded RNA (dsRNA), it uses a ribonuclease III enzyme, dicer, for processing of long dsRNA into siRNA duplexes (Bernstein, E. et al. ((2001), Nature 409: 363-366) containing ˜22 base pairs (˜2 turns of a double helix). Dicer is a bidentate RNase III, which also contains an ATP-dependent RNA helicase domain and a PAZ domain, presumably important for dsRNA unwinding and mediation of protein-protein interactions, respectively ((Bernstein, E. et al. ((2001), Nature 409: 363-366). Dicer is evolutionarily conserved in worms, flies, plants, fungi and mammals, and has a second cellular function important for the development of these organisms (Grishok, A. (2001), Cell 106:23-34; Knight, S. W. et al. (2001), Science 293:2269-2271; Hutvagner, G. et al., (2001), Science 293:834-838). At present, it is uncertain whether dicer activity in species other than D. melanogaster produces siRNAs of predominantly 21 nt in length. The estimates of siRNA size vary in the literature between 21 and 25 nt (Hamilton, A. J. et al. (1999), Science 286: 950-952; Zamore, P. D. et al. (2000), Cell 101: 25-33; Elbashir, S. M. et al., (2001), Genes Dev. 15, 188-200; Elbashir, S. M. et al. (2001), Nature 411: 494-498; Hammond, S. M. et al. (2000), Nature 404: 293-296; Hutvagner, G. et al., (2001), Science 293:834-838

The two strands of each fragment then separate enough to expose the antisense strand so that it can bind to the complementary sense sequence on a molecule of mRNA. In RNAi, a siRNA-containing endonuclease complex cleaves a single-stranded target RNA in the middle of the region complementary to the 21 nt guide siRNA of the siRNA duplex (Elbashir, S.M. et al., (2001), Genes Dev. 15, 188-200; Elbashir, S.M. et al. (2001), Nature 411: 494-498). This cleavage site is one helical turn displaced from the cleavage site that produced the siRNA from long dsRNA, suggesting dramatic conformational and/or compositional changes after processing of long dsRNA to 21 nt siRNA duplexes. The target RNA cleavage products are rapidly degraded because they either lack the stabilizing cap or poly(A) tail. A protein component of the ˜500 kDa endonuclease or RNA-induced silencing complex (RISC) was recently identified and is a member of the argonaute family of proteins (Hammond, S. M. et al. (2001) Science 293: 1146-1150), however, it is currently unclear whether dicer is required for RISC activity. Thus, the cleavage of the mRNA destroys its ability to be translated into a polypeptide. Because of their action, these fragments of RNA have been named “short (or small) interfering RNA” (siRNA).

Introducing dsRNA corresponding to a particular gene will knock out the cell's own expression of that gene. This can be done in particular tissues at a chosen time. This often provides an advantage over conventional gene “knockouts” where the missing gene is carried in the germline and thus whose absence may kill the embryo before it can be studied.

Although it has been suggested that the one disadvantage of simply introducing dsRNA fragments into a cell is that gene expression is only temporarily reduced, it has recently been shown that the system can be manipulated using a DNA vector such that the siRNA molecule can be continuously synthesized for prolonged periods of time in order to continue in suppression of the desired gene (Brummelkamp et. al. 19 Apr. 2002, Science). After two months, the cells still failed to manufacture the protein whose gene had been turned off by RNAi. Effective siRNA molecules may be designed using the following guidelines:

In general, siRNA oligonucleotides should be about 21 nucleotides in length with 2 nucleotide overhangs, usually 3′ TT.

Sequences located in the 5′ or 3′ UTR of the mRNA target and nearby the start codon should be avoided, as they may be richer in regulatory protein binding sites.

Search for a sequence AA(N19)TT or AA(N21) with approximately 50% G/C content.

Compare the selected siRNA nucleotide sequence against databases to ensure that only one gene will be targeted.

Target recognition is a highly sequence specific process, mediated by the siRNA complementary to the target. One or two base pair mismatches between the siRNA and the target gene will greatly reduce the silencing effect. It might be necessary to test several sequences since positional effects of siRNAs have been reported.

The 3′-most nucleotide of the guide siRNA does not contribute to the specificity of target recognition, while the penultimate nucleotide of the 3′ overhang affects target RNA cleavage and a mismatch reduces RNAi 2- to 4-fold. The 5′ end of the guide siRNA also appears more permissive for mismatched target RNA recognition when compared with the 3′ end. Nucleotides in the center of the siRNA, located opposite to the target RNA cleavage site, are important specificity determinants and even single nucleotide changes reduce RNAi to undetectable levels. This suggests that siRNA duplexes may be able to discriminate mutant or polymorphic alleles in gene targeting experiments, which may become an important feature for future therapeutic developments.

Double-stranded RNA has been shown to attenuate specific gene expression in C. elegans, Drosophila and Trypanosoma brucei (M. Montgomery, et al., Proc. Natl. Acad. Sci. U.S.A. 95, 15502-15507 (1998); J. Kennerdell et al., Cell 95, 1017-1026 (1998); H. Ngo et al., Proc. Natl. Acad. Sci. U.S.A. 95, 14687-14692 (1998)). The types of genes attenuated in these invertebrates include some encoding transcription factors and others that encode growth factor receptors. There is also evidence that double-stranded RNA may effectively silence gene expression in plants (M. Wassenegger et al., Plant. Mol. Biol. 37, 349-362 (1998); P. Watergiyse et al., Proc. Natl. Acad. Sci. U.S.A. 95, 13959-13964 (1998)).

A definitive mechanism through which double-stranded RNA effects gene silencing remains has not been identified (M. Montgomery et al., Trends Genet. 14, 255-258 (1998)). Recently, Montgomery et al. reported that double-stranded RNA induces specific RNA degradation in nematodes (Proc. Natl. Acad. Sci. U.S.A. 95, 15502-15507 (1998)). This conclusion was based upon the fact that DNA sequences in the targeted regions of the gene were not altered and that 100% of the F2 generation reverted to the wild type phenotype. In addition, C. elegans has a unique genetic organization. Genes in this animal are organized in operons in which a single promoter controls expression of a number of genes. They showed that the double-stranded RNA affects only expression of the targeted gene. In contrast, however, others have observed heritable effects of double-stranded RNA on the expression of a number of genes in C. elegans, suggesting that more than one mechanism may be involved in double-stranded RNA-mediated inhibition of gene activity (H. Tahara, Science 28, 431-432 (1998)).

The present invention provides a method for attenuating gene expression in a cell using gene-targeted sh RNA. The shRNA contains a nucleotide sequence that is essentially identical to the nucleotide sequence of at least a portion of the target gene, in the matter of the present invention, the RORγt genes. The cell into which the shRNA is introduced is preferably an immune cell containing at least one RORγt gene to which the shRNA is targeted. Gene expression can be attenuated in a whole organism, an organ or tissue of an organism, including a tissue explant, or in cell culture. Preferably, the cell is a mammalian cell, but the invention is not limited to mammals Double-stranded RNA (shRNA) is introduced directly into the cell or, alternatively, into the extracellular environment from which it is taken up by the cell. Inhibition is specific for the targeted gene. Depending on the particular target gene and the dose of shRNA delivered, the method may partially or completely inhibit expression of the gene in the cell. The expression of two or more genes can be attenuated concurrently by introducing two or more, shRNAs into the cell in amounts sufficient to attenuate expression of their respective target genes. shRNAs that are administered “concurrenty” are administered, together or separately, so as to be effective at generally the same time.

In yet another aspect, the invention provides a method for attenuating the expression of a RORγt gene in a cell that includes annealing two complementary single stranded RNAs in the presence of potassium chloride to yield double stranded RNA; contacting the double stranded RNA with RNAse to purify the double stranded RNA by removing single stranded RNA; and introducing the purified double stranded RNA into the cell in an amount sufficient to attenuate expression of the target gene, e.g. the RORγt gene.

The present invention provides a method for gene silencing in organisms and cells, especially mammals, using gene-specific double-stranded RNA. The ability to use double-stranded RNA to specifically block expression of particular genes in a multicellular setting both in vivo and in vitro has broad implications for the study of numerous diseases, in the matter of the present invention, inflammatory disease or conditions and autoimmune diseases or conditions.

The method of the present invention allows for attenuation of gene expression in a cell. “Attentuation of gene expression” can take the form of partial or complete inhibition of gene function. Mechanistically, gene function can be partially or completely inhibited by blocking transcription from the gene to mRNA, or by blocking translation of the mRNA to yield the protein encoded by the gene, although it should be understood that the invention is not limited to any particular mechanism of attenuation of gene expression. Inhibition of gene function is evidenced by a reduction or elimination, in the cell, of the activity associated with the protein encoded by the gene. Whether and to what extent gene function is inhibited can be determined using methods known in the art. For example, in many cases inhibition of gene function leads to a change in phenotype which is revealed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Attenuation of gene expression can be quantified, and the amount of attenuation of gene expression in a treated cell compared to a cell not treated according to the present invention can be determined. Lower doses shRNA may result in inhibition in a smaller fraction of cells, or in partial inhibition in cells. In addition, attenuation of gene expression can be time-dependent; the longer the period of time since the administration of the shRNA, the less gene expression may be attenuated. Attenuation of gene expression can occur at the level of transcription (i.e., accumulation of mRNA of the targeted gene), or translation (i.e., production of the protein encoded by the targeted gene). For example, mRNA from the targeted gene can be detected using a hybridization probe having a nucleotide sequence outside the region selected for the inhibitory double-stranded RNA, and translated polypeptide encoded by the target gene can be detected via Western blotting using an antibody raised against the polypeptide.

Delivery of a Double Stranded RNA or shRNA into a Cell

Double stranded RNA or a small interfering RN, including shRNA, can be introduced into the cell in a number of different ways. For example, it may be conveniently administered by microinjection; other methods of introducing nucleic acids into a cell include bombardment by particles covered by the dsRNA, soaking the cell or organism in a solution of the dsRNA, electroporation of cell membranes in the presence of the dsRNA, liposome-mediated delivery of dsRNA and transfection mediated by chemicals such as calcium phosphate, viral infection, transformation, and the like. The dsRNA may be introduced along with components that enhance RNA uptake by the cell, stabilize the annealed strands, or otherwise increase inhibition of the target gene. In the case of a cell culture or tissue explant, the cells are conveniently incubated in a solution containing the dsRNA or shRNA or lipid-mediated transfection; in the case of a whole animal or plant, the dsRNA or shRNA is conveniently introduced by injection or perfusion into a cavity or interstitial space of an organism, or systemically via oral, topical, parenteral (including subcutaneous, intramuscular and intravenous administration), vaginal, rectal, intranasal, ophthalmic, or intraperitoneal administration. In addition, the dsRNA or shRNA can be administered via an implantable extended release device. Methods for oral introduction include direct mixing of RNA with food of the organism, as well as engineered approaches in which a species that is used as food is engineered to express an RNA, then fed to the organism to be affected. The dsRNA may be sprayed onto a plant or a plant may be genetically engineered to express the RNA in an amount sufficient to kill some or all of a pathogen known to infect the plant.

Alternatively, dsRNA or shRNA can be supplied to a cell indirectly by introducing one or more vectors that encode both single strands of a dsRNA or shRNA (or, in the case of a self-complementary RNA, the single self-complementary strand) into the cell. Preferably, the vector contains 5′ and 3′ regulatory elements that facilitate transcription of the coding sequence. Single stranded RNA is transcribed inside the cell, and, presumably, double stranded RNA forms and attenuates expression of the target gene. Methods for supplying a cell with dsRNA by introducing a vector from which it can be transcribed are set forth in WO 99/32619 (Fire et al., published 1 Jul. 1999). A transgenic animal that expresses RNA from such a recombinant construct may be produced by introducing the construct into a zygote, an embryonic stem cell, or another multipotent cell derived from the appropriate organism. A viral construct packaged into a viral particle would accomplish both efficient introduction of an expression construct into the cell and transcription of RNA encoded by the expression construct.

The dsRNA or shRNA is typically administered in an amount that allows delivery of at least one copy per cell. The amount of dsRNA or shRNA administered to a cell, tissue, or organism depends on the nature of the cell, tissue, or organism, the nature of the target gene, and the nature of the dsRNA or shRNA, and can readily be optimized to obtain the desired level of gene inhibition. To attenuate gene expression in a single cell embryo, for example, at least about 0.8×10⁶ molecules of dsRNA are injected; more preferably, at least about 20×10⁶ molecules of dsRNA are injected; most preferably, at least about 50×10⁶ molecules of dsRNA are injected. The amount of dsRNA injected into a single cell embryo is, however, preferably at most about 1000×10⁶ molecules; more preferably, it is at most about 500×10⁶ molecules, most preferably, at most about 100×10⁶ molecules. In the case of administration of dsRNA to a cell culture or to cells in tissue, by methods other than injection, for example by soaking, electroporation, or lipid-mediated transfection, the cells are preferably exposed to similar levels of dsRNA in the medium. For example, 8-10 μL of cell culture or tissue can be contacted with about 20×10⁶ to about 2000×10⁶molecules of dsRNA, more preferably about 100×10⁶to about 500×10⁶molecules of dsRNA, for effective attenuation of gene expression.

Once the minimum effective length of the dsRNA or shRNA has been determined, it is routine to determine the effects of dsRNA or shRNA agents that are produced using synthesized oligoribonucleotides. The administration of the dsRNA or shRNA can be by microinjection or by other means used to deliver nucleic acids to cells and tissues, including culturing the tissue in medium containing the dsRNA.

The small interfering nucleic acid molecules, including RNA molecules, such as those including the shRNAs described herein, may be used for the treatment or prevention of disease. To treat or prevent a disease or other pathology, a target gene is selected which is required for initiation or maintenance of the disease/pathology. The shRNA can be introduced into the organism using in vitro, ex vivo or by in vivo methods. In an in vitro method, the shRNA is introduced into a cell, which may or may not be a cell of the organism, and the shRNA-containing cell is then introduced into the organism. In an ex vivo method, cells of the organism are explanted, the shRNA is introduced into the explanted cells, and the shRNA-containing cells are implanted back into the host. In an in vivo method, dsRNA is administered directly to the organism.

### Gene Therapy and Transgenic Vectors

A gene encoding an inhibitor of RORγt, active fragment thereof, derivative thereof, or structural/functional domain thereof, can be introduced either in vivo, ex vivo, or in vitro in a viral vector. Such vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. For example, in the treatment of neurological disorders or injuries, the striatal subventricular zone (SVZ) can be specifically targeted. Examples of particular vectors include, but are not limited to, a defective herpes virus 1 (HSV1) vector (Kaplitt et al., Molec. Cell. Neurosci., 2:320-330 (1991)), an attenuated adenovirus vector, such as the vector described by Stratford-Perricaudet et al. (J. Clin. Invest., 90:626-630 (1992)), and a defective adeno-associated virus vector (Samulski et al., J Virol., 61:3096-3101 (1987); Samulski et al., J. Virol., 63:3822-3828 (1989)) including a defective adeno-associated virus vector with a tissue specific promoter, (see e.g., U.S. Pat. No. 6,040,172, Issued Mar. 21, 2000, the contents of which are hereby incorporated by reference in their entireties).

In another embodiment the inhibitor of the RORγt gene can be introduced in a retroviral vector, e.g., as described in U.S. Pat. No. 5,399,346; Mann et al., (1983) Cell, 33:153; U.S. Pat. No. 4,650,764; U.S. Pat. No. 4,980,289; Markowitz et al., (1988) J. Virol., 62:1120; U.S. Pat. No. 5,124,263; International Patent Publication No. WO 95/07358, published Mar. 16, 1995; and Kuo et al., (1993) Blood, 82:845.

Targeted gene delivery is described in International Patent Publication WO 95/28494, published October 1995.

Alternatively, the vector can be introduced by lipofection. Liposomes may be used for encapsulation and transfection of nucleic acids in vitro. Synthetic cationic lipids designed to limit the difficulties and dangers encountered with liposome mediated transfection can be used to prepare liposomes for in vivo transfection of a gene encoding RORγt or an inhibitor thereof (Feigner, et. al., Proc. Natl. Acad. Sci. U.S.A., 84:7413-7417 (1987); see Mackey, et al., Proc. Natl. Acad. Sci. U.S.A., 85:8027-8031 (1988)). The use of cationic lipids may promote encapsulation of negatively charged nucleic acids, and also promote fusion with negatively charged cell membranes (Feigner and Ringold, Science, 337:387-388 (1989)). The use of lipofection to introduce exogenous genes into the specific organs in vivo has certain practical advantages. Molecular targeting of liposomes to specific cells represents one area of benefit. It is clear that directing transfection to particular cell types would be particularly advantageous in a tissue with cellular heterogeneity, such as the brain. Lipids may be chemically coupled to other molecules for the purpose of targeting (see Mackey et. al., Proc. Natl. Acad. Sci. U.S.A., 85:8027-8031 (1988)).

It is also possible to introduce the vector as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, e.g., Wu et al., (1992) J. Biol. Chem., 267:963-967; Wu and Wu, (1988) J. Biol. Chem., 263:14621-14624; Hartmut et al., Canadian Patent Application No. 2,012,311, filed Mar. 15, 1990).

### Inflammatory Bowel Disease

The modulators of RORgt may be particularly effective for treating inflammatory bowel disease (IBD). Ulcerative colitis (UC) and Crohn's disease are the two major forms of idiopathic Inflammatory Bowel Disease (IBD) in humans, and are widespread and poorly understood disorders (Kirsner, J. B., et al., eds., Inflammatory Bowel Disease: 3rd ed., Lea and Febiger, Philadelphia (1988); Goldner, F. H., et al., Idiopathic Inflammatory Bowel Disease, in Stein, J. H., ed., Internal Medicine, Little Brown & Co., Boston, pp. 369-380 (1990); Cello, J. P., et al. Ulcerative Colitis, in Sleisenger, M. H., et al. eds., Gastrointestinal Disease: Pathophysiology Diagnosis Management, W. B. Saunders Co., Philadelphia, p. 1435 (1989)). Other forms of IBD include those caused by infectious agents, drugs, or the solitary rectal ulcer syndrome and collagenous colitis. The diagnosis of IBD of known and unknown etiology is difficult and sometimes impossible to make (Riddell, R. H., ed., Pathology of Drug-induced and Toxic Diseases, Churchill Livingstone, New York (1982)).

Colitis generally refers to a more superficial mucosal disease in contrast to Crohn's disease, which presents as a deep, often transmucosal involvement and fissures (Riddell, R. H., ed., Pathology of Drug-induced and Toxic Diseases, Churchill Livingstone, New York (1982); Morrison, B. C., et al. eds., Gastrointestinal Pathology, 2d ed., London (1979); Fenoglio-Preiser, C. M., et al., eds., Gastrointestinal Pathology: An Atlas and Text, Raven Press, New York (1989); Goldman, H., et al., Hum. Pathol. 13:981-1012 (1982)). Ulcerative colitis typically involves the rectum and extends proximally without intervening uninvolved areas. These uninvolved areas are usually the hallmark of Crohn's disease. The histologic features of active ulcerative colitis include, beside the superficial ulcers, infiltration by inflammatory cells (e.g., mainly lymphocytes, plasma cells, variable number of neutrophils, eosinophils and mast cells) involving extensively the lamina propria. Crypt abscesses, which are aggregates of neutrophils near and invading the crypt epithelium, are generally reliable indicators of activity, while depletion of mucin in goblet cells is a less frequent finding. Foreign-body giant cells and collection of a few histiocytes, however, may be present due to the rupture of crypt abscesses and the spilling of mucin into the submucosa, which often elicits a cellular reaction. Noncaseating granulomas, may be present in gut segments from Crohn's disease, which is often also called granulomatous colitis.

The etiology and pathogenesis of idiopathic IBD, as the name implies, are poorly understood. Numerous theories, however, implicate genetic predisposition, environmental factors, infectious agents and immunologic alterations (Kirsner, J. B., et al. eds., Inflammatory Bowel Disease, 3rd ed., Lea and Febiger, Philadelphia (1988); Zipser, R. D., ed., Dig. Dis. Sci., 33 Suppl.:1S-87S (1988)).

Eliakim et al. have demonstrated enhanced production of platelet-activating factor (PAF) during active disease and inhibition by sulfasalazine and prednisolone (Eliakim, R., et al., Gastroenterology 95:1167-1172 (1988)), thus implicating PAF as a possible mediator in the disease process. Furthermore, an enhanced synthesis of eicosanoids such as prostaglandins, thromboxanes and leukotrienes has been shown in both human and experimental IBD (Schumert, R., et al., Dig. Dis. Sci. 33 Suppl.:58S-64S (1988)). These products may be involved in the pathogenesis of IBD. Selective inhibition of leukotrienes may be a therapeutic strategy to reduce inflammation in IBD (Schumert, R., et al., Dig. Dis. Sci. 33 Suppl.:58S-64S (1988); Goetzl, E. J., et al., Dig. Dis. Sci. 33 Suppl.:36S-40S (1988); Allgayer, H., et al., Gastroenterology 96:1290-1300 (1989)).

Potential humoral mediators of inflammation may also be involved in the pathogenesis of IBD, e.g., tumor necrosis factor, growth factors, neuropeptides, lipoxins, and mast cell products (Zipser, R. D., ed., Dig. Dis. Sci., 33 Suppl.:IS-87S (1988); Shanahan, F., et al., Dig. Dis. Sci. 33 Suppl.:41S-49S (1988); Nast, C. C., et al., Dig. Dis. Sci 33 Suppl.:50S-57S (1988); Mayer, E. A., et al., Dig. Dis. Sci. 33 Suppl.:71S-77S (1988)). It is also possible that not only the number of inflammatory cells and their products are changed, but the number of receptors increase, such as the increased neutrophil receptors for and response to the proinflammatory peptide formyl-methionyl-leucyl-phenylalanine (FMLP) (Anton, P. A., et al., Gastroenterology 97:20-28 (1989)) and the adherence of leukocytes (Cason, J., et al., J. Clin. Pathol. 41:241-246 (1988)) in Crohn's disease.

The immunologic alterations in IBD are primarily autoimmune in nature, with colonic autoantibodies and lymphocyte-cytotoxicity directed against colonic epithelial cells. There are many animal models utilized to study the etiology and pathogenesis of IBD. The criteria for an animal model of IBD have been reviewed (Strober, W., Dig. Dis. Sci. 33 Suppl.:3S-1OS (1988); Beekan, W. L., Experimental inflammatory bowel disease, in: Kirsner, J. B., et al., eds., Inflammatory Bowel Disease, Lea and Febiger, Philadelphia, pp. 37-49 (1988)). The available animal models can be divided into naturally occurring and experimentally induced IBD animal models. Only a few spontaneous and rarely occurring models of intestinal inflammation due to a genetic defect are available and most of these are not idiopathic but are induced by bacteria or other infectious agents (e.g., hyperplasia, crypt abscesses, ulcers in mice with Bacillus psyliformnis and hamster with “rod-shaped bacteria”) (Strober, W., Dig. Dis. Sci. 33 Suppl.:3S-1OS (1988)). Rare forms of spontaneous ulcerative colitis and granulomatous enterocolitis also occur in rats and horses, respectively.

Experimentally induced animal models of ulcerative colitis are usually produced by exposure to toxic dietary substances, pharmacologic agents or other environmental chemicals, or by administration of materials derived from patients, or by manipulation of the animal's immune system (Strober, W., Dig. Dis. Sci. 33 Suppl.:3S-10S (1988); Beekan, W. L., Experimental inflammatory bowel disease, in: Kirsner, J. B., et al., eds., Inflammatory Bowel Disease, Lea and Febiger, Philadelphia, pp. 37-49 (1988); Onderdonk, A. B., Dig. Dis. Sci. 33 Suppl.:40S-44S (1988)).

The most widely used models are the experimental colonic lesions produced by dinitrobenzene sulfonic acid (DNBS), 2, 4, 6-trinitro-benzensulfonic acid (TNBS) and carrageenan. These models involve tissue destruction in the colon. Intrarectal administration of 5-30 mg of TNBS in 0.25 ml of 50% ethanol in the rat produces dose-dependent colonic ulcers and inflammation which are observed by gross and light microscopic examination, and by biochemical measurement of myeloperoxidase activity in the colon at 3-4 weeks (Morris, G. P., et al., Gastroenterology 96:795-803 (1989)). Histologically, the inflammatory infiltrate of mucosa and submucosa included polymorphonuclear leukocytes, lymphocytes, macrophages and connective tissue mast cells. Initially, massive edema and in the healing state (6-8 weeks) fibroblasts are also detected. Granulomas are also seen in 57% of rats killed at 3 weeks.

Carrageenan is a sulfated polygalactose (molecular weight above 100,000) widely used in the food industry and is considered safe for human use. Degraded forms of this polysaccharide (molecular weight 20,000-40,000) administered through drinking water induce ulcerative colitis in two weeks or later in experimental animals (Beekan, W. L., Experimental inflammatory bowel disease, in: Kirsner, J. B., et al., eds., Inflammatory Bowel Disease, Lea and Febiger, Philadelphia, pp. 37-49 (1988); Onderdonk, A. B., Dig. Dis. Sci. 33 Suppl.:40S-44S (1988); Benitz, K. F., et al., Food Cosmet. Toxicol. 11:565 (1973); Engster, M., et al., Toxicol. Appl. Pharmacol. 38:265 (1976)). In addition to ulcers, acute and chronic inflammation, macrophages laden with degraded carrageenan and suppressed phagocytosis are seen.

In addition to carrageenan, the FMLP-induced experimental colonic lesions also represent a transition between chemically and cellularly induced animal models. This bacterial peptide activates and attracts neutrophils, and causes ulcers and inflammation in the rat ileum (VonRitter, C., et al., Gastroenterology 95:651-656 (1988); VonRitter, C., et al., Gastroenterology 96:811-816 (1989)). This new animal model, like the TNB, has not yet been extensively used.

Szabo proposed a new model for ulcerative colitis, which incorporates the administration of a sulfhydryl blocker, such as N-ethylmaleimide, iodoacetamide, iodoacetate or chloroacetate (U.S. Pat. No., 5,214,066), to the intestinal mucosa of animals. Delivery of these agents to the colon of rodents resulted in chronic ulcerative colitis.

### Multiple Sclerosis

Another inflammatory disease that may respond to treatment with a modulator of RORγt is multiple sclerosis. MS is a multi-factorial inflammatory disease of the human central nervous system resulting in the slowing of electrical conduction along the nerve. The disease is characterized by an increase in the infiltration of inflammatory cells, loss of oligodendrocytes, and increased gliosis (astrocyte hypertrophy and proliferation). (For review see Amit et al., 1999; Pouly et al., 1999; Steinman et al., 1993; Miller, 1994). Myelin is the target of this cellular autoimmune inflammatory process, leading to impaired nerve conduction (for a review, see e.g. Thompson 1996, Clin. Immunother. 5, 1-11). Clinical manifestations are variable, but are usually characterized by an initial relapsing-remitting course, with acute exacerbation followed by periods of clinical stability. Over time, a steady deterioration in neurological functions takes place as the disease evolves into a chronic progressive phase. This deterioration is responsible for disabling complications and side-effects, which greatly affect quality of life and increases mortality risk of affected patients. It is estimated that close to a third of a million people in the United States have MS.

There are several models that are widely used for testing therapies that may be effective in treating MS. One model is the Experimental Allergic Encephalomyelitis (EAE) model. EAE is a T cell mediated autoimmune disease of the central nervous system (CNS). Disease can be induced in susceptible strains of mice (SJL mice) by immunization with CNS myelin antigens or alternatively, disease can be passively transferred to susceptible mice using antigen stimulated CD4+ T cells (Pettinelli, J. Immunol 127, 1981, p. 1420). EAE is widely recognized as an acceptable animal model for multiple sclerosis in primates (Alvord et al. (eds.) 1984. Experimental allergic encephalomyelitis--A useful model for multiple sclerosis. Alan R. Liss, New York). Another commonly utilized experimental MS model is a viral model, whereby an MS like disease is induced by Theiler's murine encephalomyelitis virus (TMEV) (Dal Canto, M. C., and Lipton, H. L., Am. J. Path., 88:497-500 (1977)). Additionally, the lysolecithin model is widely accepted as a model for demyelinating conditions such as MS.

Moreover, Example 6 describes the use of the EAE model to determine the role of RORγt in this model of multiple sclerosis. The results show that mice deficient in RORγt develop a significantly less severe form of the disease, as compared to wild type mice, thus pointing to a role for RORγt in the development and/or progression of the disease.

### Arthritis

It is also possible that modulators of RORγt may be used to treat arthritis, both rheumatoid arthritis and osteoarthritis.

Rheumatoid arthritis (RA) is a chronic, systemic and articular inflammatory disorder which is characterized as an imbalance in the immune system that causes an overproduction of pro-inflammatory cytokines, e.g., tumor necrosis factor alpha (TNFa), interleukin 1 (IL-1), and a lack of anti-inflammatory cytokines, e.g. IL-10, IL-11. RA is characterized by synovial inflammation, which progresses to cartilage destruction, bone erosion and subsequent joint deformity. The primary symptoms of RA are joint inflammation, stiffness, swelling, fatigue, difficulty moving, and pain. During the inflammatory process, polymorphonuclear cells, macrophages, and lymphocytes are released. Activated T-lymphocytes produce cytotoxins and pro-inflammatory cytokines, while macrophages stimulate the release of prostaglandins and cytotoxins. Vasoactive substances (histamine, kinins, and prostaglandins) are released at the site of inflammation and cause edema, warmth, erythema, and pain associated with inflamed joints.

The pathogenesis of rheumatoid arthritis, leading to the destruction of the joints, is characterized by two phases: 1) an exudative phase involving the microcirculation of the synovial cells that allow an influx of plasma proteins and cellular elements into the joint and 2) a chronic inflammatory phase occurring in the sub-synovium and sub-chondral bone, characterized by pannus (granulation tissue) formation in the joint space, bone erosion, and cartilage destruction. The pannus may form adhesions and scar tissue which causes the joint deformities characteristic of rheumatoid arthritis.

The etiology of rheumatoid arthritis remains obscure. Infectious agents such as bacteria and viruses have been implicated.

Current rheumatoid arthritis treatment consists predominantly of symptomatic relief by administration of non-steroidal anti-inflammatory drugs (NSAIDs). NSAID treatment is mainly effective in the early stages of rheumatoid arthritis; it is unlikely it will produce suppression of joint inflammation if the disease is present for more than one year. Gold, methotrexate, immunosuppressants and corticosteroids are also used.

Osteoarthritis is a disorder of the movable joints characterized by deterioration and abrasion of articular cartilage, as well as by formation of new bone at the joint periphery and usually presents as pain, which worsens with exercise, or simply an X-ray that clearly shows thinning cartilage. Common joints affected are the knees, hips and spine, finger, base of thumb and base of the big toe. Osteoarthritis is characterized by degenerative changes in the articular cartilage (the supporting structure) and subsequent new bone formation at the articular margins. As osteoarthritis progresses, the surface of the articular cartilage is disrupted and wear-particles gain access to the synovial fluid which in turn stimulates phagocytosis by macrophage cells. Thus, an inflammatory response is eventually induced in osteoarthritis. Common clinical symptoms of osteoarthritis include cartilaginous and bony enlargements of the finger joints and stiffness on awakening and painful movement.

There is no definitive answer regarding the cause of osteoarthritis. A natural erosion of cartilage occurs with age, but excessive loads placed on joints, obesity, heredity, trauma, decreased circulation, poor bone alignment, and repetitive stress motion play a role. Osteoarthritis may also be the result of free radical damage, thought to be a major cause of many diseases, including the aging process, cancer, heart disease and degenerative diseases.

There is no known drug that claims to reverse osteoarthritis. Most therapeutic agents are directed at reducing the inflammation and relieving pain. Non-steroidal anti-inflammatory drugs (NSAIDs) are the first line of treatment for osteoarthritis. Other treatments include disease-modifying arthritic drugs (“DMARDs”), steroids, and physical therapy.

One of the models used to test for new therapies for arthritis includes the collagen-induced arthritis model (CIA) (Myers, L. K. et al. Life Sci. (1997), 61(19): 1861-1878). In this model, immunization of genetically susceptible rodents or primates with Type II collagen (CII) leads to the development of a severe polyarticular arthritis that is mediated by an autoimmune response. It mimics RA in that synovitis and erosions of cartilage and bone are the hallmarks of CIA.

### Diabetes

It is also possible that modulators of RORγt may be used to treat diabetes. Modulators of RORγt may be particularly useful in treating insulin-dependent diabetes mellitus (IDDM). The main clinical feature of IDDM is elevated blood glucose levels (hyperglycemia). The elevated blood glucose level is caused by auto-immune destruction of insulin-producing β-cells in the islets of Langerhans of the pancreas (Bach et al. 1991, Atkinson et al. 1994). This is accompanied by a massive cellular infiltration surrounding and penetrating the islets (insulitis) composed of a heterogeneous mixture of CD4+ and CD8+ T-lymphocytes, B-lymphocytes, macrophages and dendritic cells (O'Reilly et al. 1991).

One animal model that is particularly useful in testing agents for treating IDDM is the NOD mouse. The NOD mouse represents a model in which auto-immunity against beta-cells is the primary event in the development of IDDM. Diabetogenesis is mediated through a multi-factorial interaction between a unique MHC class II gene and multiple, unlinked, genetic loci, as in the human disease. Moreover, the NOD mouse demonstrates beautifully the critical interaction between heredity and environment, and between primary and secondary auto-immunity. Its clinical manifestation is, for example, depending on various external conditions, most importantly on the micro-organism load of the environment in which the NOD mouse is housed.

Another animal model for studying the effects of therapeutic agents in IDDM is the streptozotocin (STZ) model (Hartner, A. et al. (2005), BMC Nephrol. 6(1):6). This model has been used extensively as an animal model to study the mechanisms involved in the destruction of pancreatic beta cells in IDDM. In this model, diabetes is induced in rodents by the beta-cell toxin streptozotocin (STZ). STZ is taken up by the pancreatic beta cell through the glucose transporter GLUT-2. This substance decomposes intracellularly, and causes damage to DNA either by alkylation or by the generation of NO. The appearance of DNA strand breaks leads to the activation of the abundant nuclear enzyme poly(ADP-ribose) polymerase (PARP), which synthesizes large amounts of the (ADP-ribose) polymer, using NAD+ as a substrate. As a consequence of PARP activation, the cellular concentration of NAD+ may then decrease to very low levels, which is thought to abrogate the ability of the cell to generate sufficient energy and, finally, to lead to cell death.

### Use of RORγt Modulators for Treatment of Cancer

### Cancer Treatment and Vaccines

While the inventors have proposed that modulators of RORγt, particularly antagonists of RORγt may be used to downregulate the inflammatory response in many immune related diseases or conditions, they have also proposed that agonists or stimulators of RORγt may be used in situations whereby upregulation of the immune response is desirable. Any organ or tissue in which a tumor may arise may respond to therapy with an agonist or stimulator of RORγt, since the presence/expression of RORγt is associated with certain population of lymphoid cells that may act to directly inhibit tumor cell proliferation or may act indirectly to stimulate or activate anti-tumor T or B lymphocyte responses. Accordingly, it may be possible to identify an agent that stimulates the expression of RORγt as described herein that may be further tested in appropriate tumor models. While the agonists of RORγt may be useful to upregulate the immune response to any tumor antigen, tumors of the intestinal tract may be of particular interest given the results of the studies described herein.

For example, colorectal cancer (CRC) is one of the leading cancer forms in the Western world (1.3 million per year and over 600,000 annual deaths). The great majority of CRC cases are sporadic cancers, for which it is not possible to establish a genetic disposition. Effective CRC prevention in well-defined risk groups would have a significant effect on population health. In recent years, focus is very much on cancer prophylaxis, in acknowledgement of the fact that surgery mostly does not suffice as the only modality and that most cytotoxic regimens are ineffective against solid tumors. The term chemoprophylaxis covers the use of pharmacologically active, non-cytotoxic agents or naturally occurring nutrients that protect against the emergence and development of clones of mutated, malignant cells.

Another area of great interest is in the development of tumor cell vaccines. Tumor cells are known to express tumor-specific antigens on the cell surface. These antigens are believed to be poorly immunogenic, largely because they represent gene products of oncogenes or other cellular genes which are normally present in the host and are therefore not clearly recognized as nonself. Although numerous investigators have tried to target immune responses against epitopes from various tumor specific antigens, none have been successful in eliciting adequate tumor immunity in vivo (Mocellin S., (2005), Front Biosci. 10:2285-305).

The inventors of the present application have proposed that a modulator of RORγt, particularly an agonist or stimulator of RORγt may aid in development of appropriate immune responsiveness to the tumor antigens prevalent in the cancerous condition. Models for assessment of humoral and cell mediated responses to tumor antigens are well known to those skilled in the art.

### EXAMPLES

### Example 1

### Development of Animal Model and Studies on Lymphoid Cells in These Animals Materials and Methods

### Mice

The generation of gene-targeted Rorc(γt)^{+/GFP} and Rorc(γt)^{GFP/GFP} (G. Eberl et al. (2004), Nat. Immunol 5: 64), and BAC transgenic mice Rorc(γt)-Bcl-xl-IRES-EYFP^{Tg} (T. Sparwasser et al. (2004), Genesis 38: 39) have been described recently. The Rorc(γt)-Cre^{Tg} BAC-transgenic mice were generated following the same protocol. Id2-deficient (Yokota et al. (1999), Nature 397: 702) and R26R mice (Mao et al. (2001), Blood 97: 324) have been reported elsewhere. LTα− and Rag-2-deficient mice were purchased from The Jackson Laboratory (Bar Harbor, Me.). All mice were bred and used in the specific pathogen-free animal facility according to the New York University School of Medicine Institutional Animal Care and Use Committee.

### Antibodies

The following proteins and mAbs were purchased from Pharmingen (San Diego, Calif.): fluorescein isothiocyanate (FITC)-conjugated Annexin V, phycoerythrin (PE)-conjugated anti-CD4 (RM4-5), anti-CD11c (HL3), anti-CD8β (53-5.8), anti-CD44 (IM7), anti-CD49b (DX5), anti-ICAM-1 (3E2), anti-c-kit (2B8), anti-NK1.1 (PK136), anti-TCRβ (H57-597), allophycocyanin (APC)-conjugated anti-CD3ε (145-2C11), anti-CD11b (M1/70), anti-CD11c (HL3), anti-B220 (RA3-6B2), anti-Gr-1 (RB6-8C5), biotin-conjugated anti-CD8α (53-6.7), anti-CD45.2 (104), anti-VCAM-1 (429), anti-TCRδ (GL3), and purified anti-CD16/32 (2.4G2). Rabbit anti-GFP, FITC-conjugated goat anti-rabbit, Cy3-conjugated goat anti-Armenian hamster and Alexa Fluor 647-conjugated streptavidin were purchased from Molecular Probes (Eugene, Oreg.). Biotin-conjugated anti-IL-7Rα mAb was purchased from eBioscience (San Diego, Calif.). The PE-conjugated anti-mouse IL-17 antibody was purchased from BD Pharmingen. The mouse anti-CD3PerCP (145-2C11) and anti-mouse CD28 (37.51) antibodies were purchased from BD Pharmingen. The hamster monoclonal antibody to murine RORγ and RORγt was prepared at the Sloan Kettering Cancer Center monoclonal core facility. Briefly, animals were immunized with a His-tagged RORγ expressed in bacteria, and hybridoma supernatants were screened by ELISA on a MBP-RORγ fusion protein. Supernatants of positive clones were further screened for immunoblot reactivity with RORγ in extracts from RORγ-transfected 293T cells and for immunofluorescence staining of thymic sections Immunohistochemical localization of proteins was performed by incubating the slides in the presence of primary antibodies diluted in PBS, 0.1% Triton, 1% heat inactivated goat serum (HINGS) overnight at 4° C. Then sections were rinsed with PBS, 1% HINGS, and incubated with secondary antibodies 30 min at RT, rinsed in PBS, and cover slipped using Vectashield mounting medium (Vector Laboratories).

### Flow Cytometry

Single cell suspensions were prepared from thymus, spleen and Peyer's patches. Small intestinal mononuclear cells were prepared as follows. Peyer's patches were removed, the intestine was cut into pieces less than 1 mm³, and incubated 1 hour at 37° C. in 15ml DMEM containing lmg/ml collagenase D (Roche Diagnostics, Mannheim, Germany). Total intestinal cells were resuspended in a 40% isotonic Percoll solution (Pharmacia, Uppsala, Sweden) and underlaid with an 80% isotonic Percoll solution. Centrifugation for 20 min at 2000 rpm yielded the mononuclear cells at the 40-80% interface. Cells were washed twice with PBS-F (PBS containing 2% fetal calf serum, FCS), preincubated with mAb 2.4G2 to block Fcy receptors, then washed and incubated with the indicated mAb conjugates for 40 min in a total volume of 100 μl PBS-F. Cells were washed, resuspended in PBS-F and analyzed on a FACScalibur flow cytometer (Becton-Dickinson, San Jose, Calif.). For cell cycle analysis of thymocytes, cells were fixed in 70% ethanol 30 min at 4° C., washed with PBS-F, and 5×10⁵ cells were incubated 5 min at 37° C. with 12.5 μg/ml of propidium iodide (Sigma) and 50 μg/ml of RNAse A in 100 μl STE buffer (100 mM Tris base, 100 mM NaCl and 5 mM EDTA at pH7.5). Cells were then washed, resuspended in PBS-F and analyzed.

### Thymocyte Survival Assay

Thymocytes were isolated and cultured in DMEM medium supplemented with DMEM containing 10% FCS, 10 mM HEPES, 50 μM -mercaptoethanol, and 1% glutamine. After the indicated periods of time, cells were stained with Annexin V (Pharmingen) and 1 μg/ml of propidium iodide to exclude dead cells, and analyzed by FACS.

### Immunofluorescence Histology

Adult intestines were washed several hours in PBS before being fixed overnight at 4° C. in a fresh solution of 4% paraformaldehyde (Sigma, St-Louis, Mo.) in PBS. The samples were then washed 1 day in PBS, incubated in a solution of 30% sucrose (Sigma) in PBS until the samples sank, embeded in OCT compound 4583 (Sakura Finetek, Torrance, Calif.), frozen in a bath of hexane cooled with liquid nitrogen and stocked at −80° C. Blocs were cut with a Microm HM500 OM cryostat (Microm, Oceanside, Calif.) at 8 μm (tissues) thickness and sections collected onto Superfrost/Plus slides (Fisher Scientific, Pittsburgh, Pa.). Slides were dried 1 hour and processed for staining, or stocked at −80° C. For staining, slides were first hydrated in PBS-XG, (PBS containing 0.1% triton X-100 and 1% normal goat serum, Sigma) for 5 min and blocked with 10% goat serum and 1/100 of anti-Fc receptor mAb 2.4G2 in PBS-XG for 1 hour at room temperature. Endogenous biotin was blocked with a biotin blocking kit (Vector Laboratories, Burlingame, Calif.). Slides were then incubated with primary polyclonal Ab or conjugated mAb (in general 1/100) in PBS-XG overnight at 4° C., washed 3 times 5 min with PBS-XG, incubated with secondary conjugated polyclonal Ab or streptavidin for 1 hour at room temperarture, washed once, incubated with 4′6-diamidino-2-phenylindole-2HCl (DAPI) (Sigma) 5 min at room temperature, washed 3 times 5 min and mounted with Fluoromount-G (Southern Biotechnology Associates, Birmingham, Ala.). Slides were examined under a Zeiss Axioplan 2 fluorescence microscope equipped with a CCD camera and processed with Slidebook v3.0.9.0 software (Intelligent Imaging, Denver, Colo.).

### Results

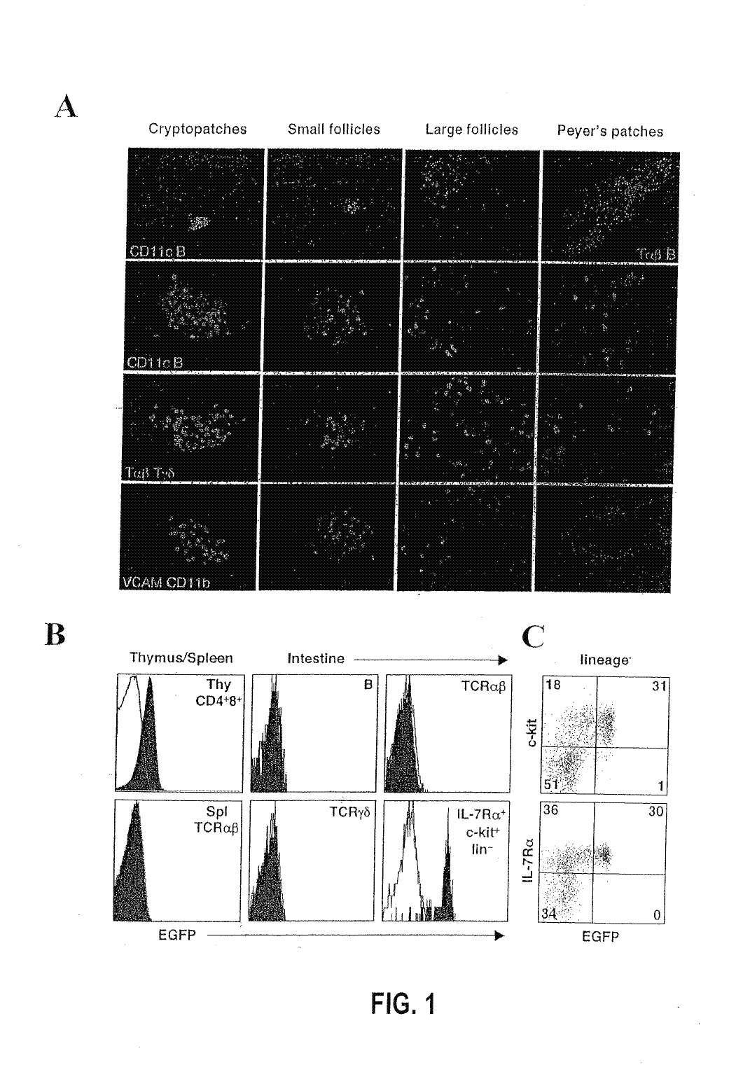

The nuclear retinoic acid related orphan receptor RORγt is necessary for the development of LNs and PPs (Sun, Z. et al., (2000) Science 288:2369; Eberl, G. et al. (2004), Nat. Immunol. 5:64). During fetal life, RORγt is exclusively expressed in lymphoid tissue inducer (LTi) cells and is required for the generation of these cells (Eberl, G. et al. (2004), Nat. Immunol 5:64). In the adult, RORγt regulates the survival of double positive (DP) CD4⁺CD8⁺ immature thymocytes (Sun, Z. et al., (2000) Science 288:2369). Using mice that are heterozygous for insertion of a green fluorescent protein (GFP) reporter into the Rorc(γt)gene (Rorc(γt)^{+/GFP} mice) (Eberl, G. et al. (2004), Nat. Immunol 5:64)), it was determined that, in adult animals, RORγt is expressed in a third type of cells, namely the cryptopatch (CP) cells (FIG. 1A). RORγt⁺ cells were also found in isolated lymphoid follicles (ILFs) and in the sub-epithelial dome of PPs, but not within the intestinal epithelium or in mLNs or in periaortic LNS. Most, if not all, intestinal RORγt⁺ cells expressed both c-kit and IL-7Rα, and all lin⁻c-kit⁺IL-7Rα⁺ cells expressed RORγt (FIGS. 1B and 1C).

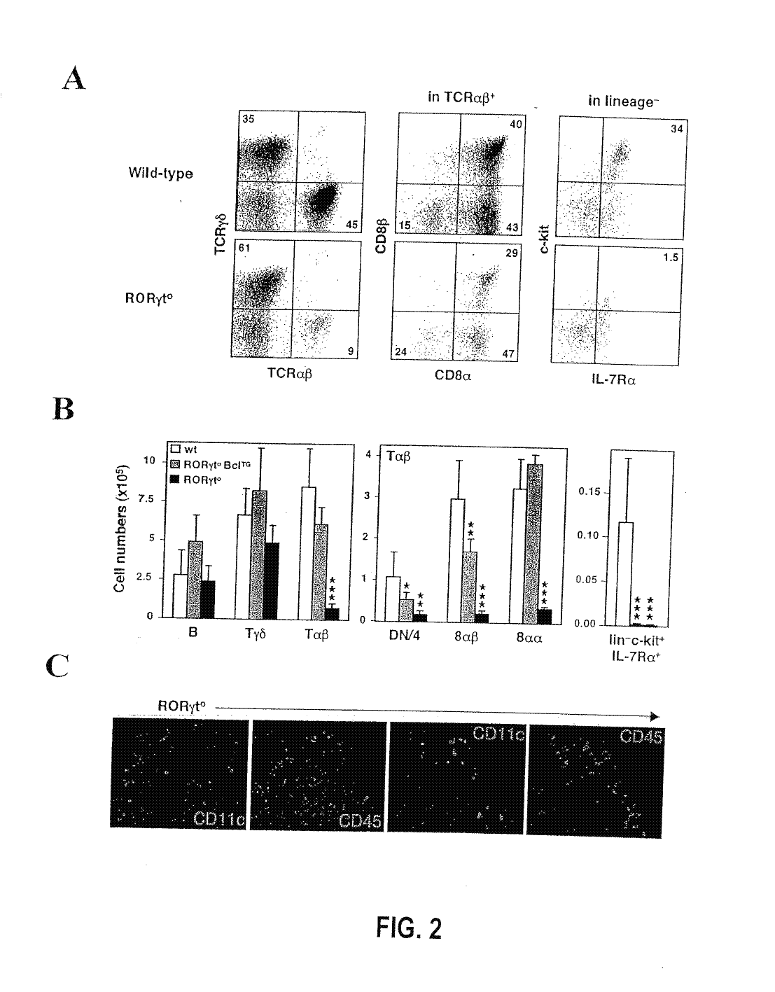

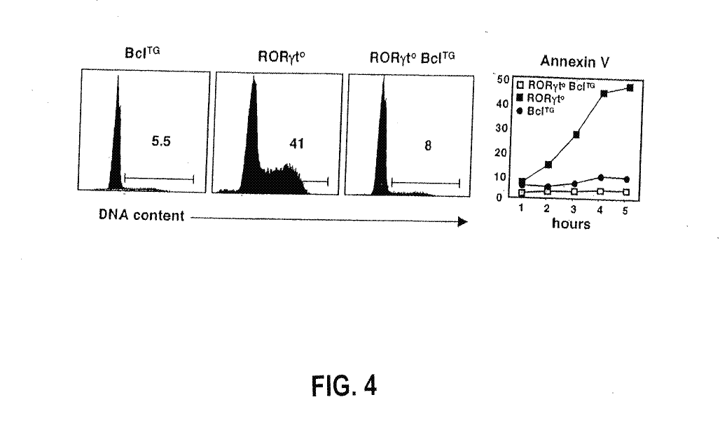

In mice rendered deficient for RORγt through breeding the Rorc(γt)^{GFP} allele to homozygosity, intestinal lin⁻c-kit⁺IL-7Rα⁺ cells and CPs were absent, and no intestinal GFP⁺ cells could be observed. In these animals, ILFs also failed to develop (FIG. 2), as apparent by the absence of B cell clusters characteristic of these structures (FIG. 1A) (Y. Kanamori et al., J Exp Med 184, 1449 (1996); K. Suzuki et al., Immunity 13, 691 (2000)). Although intestinal B cells, γδ T cells and CD11c⁺ cells (FIG. 2) were present in normal numbers in the mutant mice, there was substantial and specific reduction in all subsets of intestinal αβ T cells, including CD4⁻8⁻ (DN), CD4⁺, CD8αβ⁻, and CD8αα⁺ cells (FIG. 2B). This decrease in intestinal αβ T cells could be accounted for either by reduced thymic output (Z. Sun et al., Science 288, 2369 (2000). or by impaired differentiation of cells outside of the thymus. In the absence of RORγt, DP thymocytes progress prematurely into cell cycle and undergo massive apoptosis (Z. Sun et al., Science 288, 2369 (2000)), a phenotype that can be rescued by transgenic expression of Bcl-xL (Z. Sun et al., Science 288, 2369 (2000)). To force expression of Bcl-xL in intestinal RORγt⁺ cells, we generated bacterial artificial chromosome (BAC)-transgenic mice (X. W. Yang, P. Model, N. Heintz, Nat Biotechnol 15, 859 (1997) that express Bcl-xL under control of the Rorc(γt) gene (Rorc(γt)-Bcl-xl^{TG} mice) (T. Sparwasser, S. Gong, J. Y. H. Li, G. Eberl, Genesis 38, 39 (2004)). In RORγt-deficient mice, this transgene was able to restore normal cell cycle and survival of thymocytes (FIG. 4), but failed to restore development of intestinal lin⁻c-kit⁺IL-7Rα⁺ cells (FIG. 2B), CPs and ILFs (Data not shown). This result suggests that the mode of action of RORγt in intestinal RORγt⁺ cells is independent of Bcl-xL expression. Despite the absence of CPs and ILFs, relatively normal numbers of intestinal αβ T cells, including CD8αα⁺ TCR⁺ IEL, were recovered from the intestine of RORγt-deficient Rorc(γt)-Bcl-xl^{TG} mice (FIG. 2B). These results demonstrate that intestinal RORγt⁺ cells, i.e. lin⁻c-kit⁺IL-7Rα⁺ CP cells, are not required for development of intestinal αβ or γδ T cells.

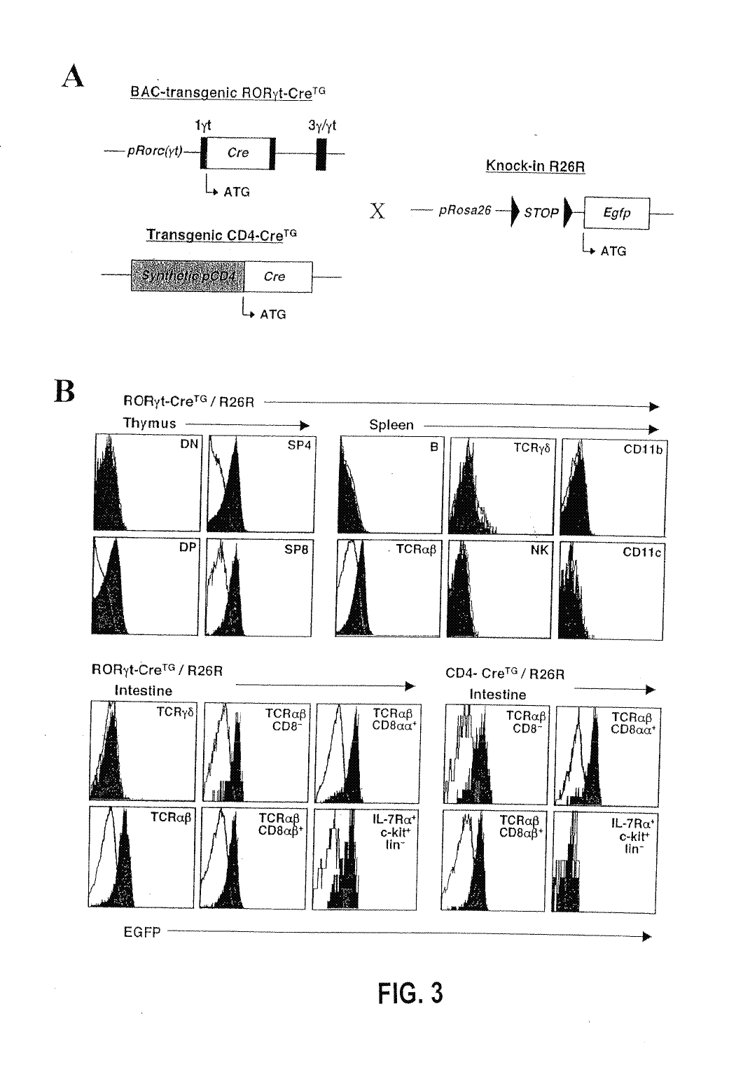

To directly determine which cells give rise to intestinal αβ T cells, we performed a genetic cell fate mapping experiment. BAC transgenic mice expressing Cre recombinase under control of the Rorc(γt) gene (Rorc(γt)-Cre^{TG} mice) were generated and bred to R26R reporter mice, which express GFP under control of the ubiquitously active gene Rosa26 after a LoxP-flanked Stop sequence is excised by Cre (X. Mao, Y. Fujiwara, A. Chapdelaine, H. Yang, S. H. Orkin, Blood 97, 324 (2001)) (FIG. 3A). Thus, in Rorc(γt)-Cre^{TG}/R26R mice, only RORγt⁺ cells and their progeny are capable of expressing GFP. In these animals, DP thymocytes and their CD4⁺ and CD8⁺ single positive (SP) progeny expressed GFP, whereas DN precursors did not (FIG. 3B). In spleen, all αβ T cells expressed GFP, which mapped them as the progeny of DP thymocytes. This was in contrast to γδ T cells, B cells, NK cells, CD11c⁺ dendritic cells, and CD11b⁺ myeloid cells, which did not express GFP (FIG. 3B, upper panel). A similar situation was observed in the intestine (FIG. 3B, lower panel), clearly demonstrating that intestinal αβ T cells were all specifically derived from RORγt⁺ cells.

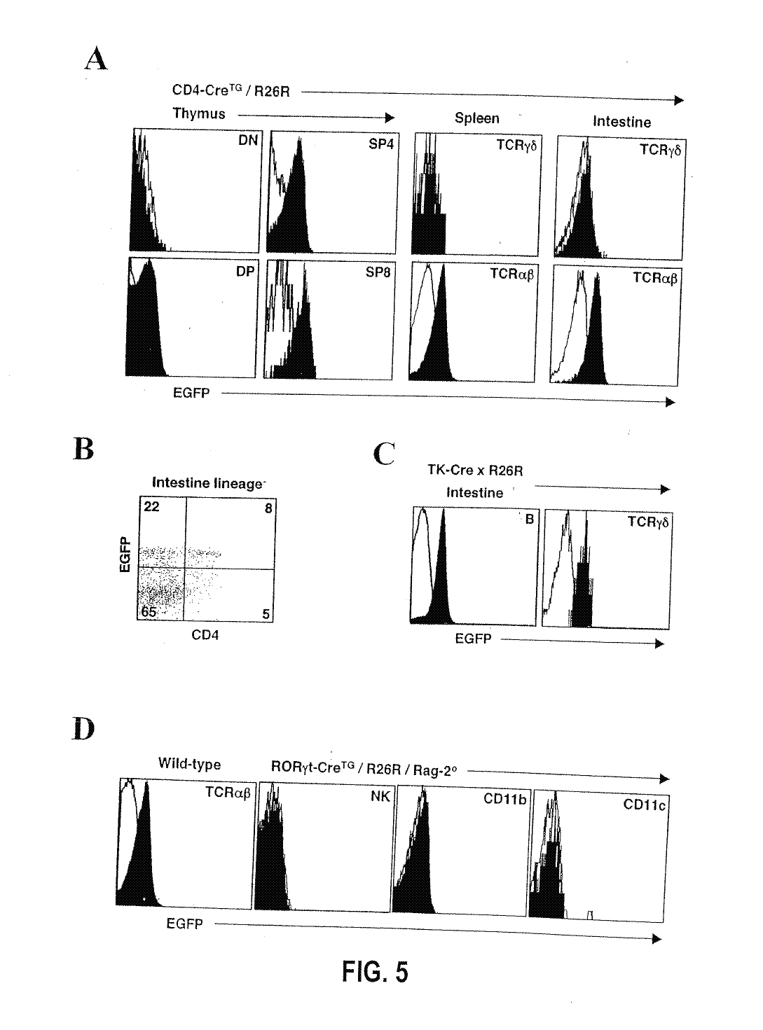

In a second cell fate mapping experiment, R26R mice were bred to transgenic mice expressing Cre under the control of murine CD4 regulatory elements (S. Sawada, J. D. Scarborough, N. Killeen, D. R. Littman, Cell 77, 917 (1994)) (Cd4-Cre^{TG} mice, FIG. 3A). In Cd4-Cre^{TG}/R26R mice, all T cells that had transited through the DP stage of thymic development, such as SP thymocytes and αβ T cells in the spleen, expressed GFP (FIG. 5A). Again, intestinal αβ T cells, but not γδ T cells or B cells, expressed GFP (FIGS. 3B and 5A). In these mice, intestinal lin⁻c-kit⁺IL-7Rα⁺ cells did not express GFP, probably because the T cell-specific minimal CD4 enhancer/promoter is not active in these cells, even though a substantial fraction of intestinal RORγt⁺ cells express CD4 (FIG. 5B). These results confirm that, rather than being the progeny of intestinal RORγt⁺ cells, intestinal αβ T cells are derived from DP thymocytes. In addition, these results shed light on the source of TCR 4 IEL that express CD8αα homodimers. These unique intestinal T cells, previously proposed to be derived from double negative thymocytes based on experiments performed with TCR-transgenic mice (D. Guy-Grand et al., Eur J Immunol 31, 2593 (2001)) are shown here to differentiate from CD4⁺CD8⁺ progenitors. A synopsis of the cell-fates derived from these mapping experiments is presented in Table S1.

The hypothesis that CPs harbor precursors of 4 and γδ IEL (H. Saito et al., Science 280, 275 (1998); K. Suzuki et al., Immunity 13, 691 (2000).) was first questioned by the finding that lin⁻c-kit⁺IL-7Rα⁺ CP cells express germline TCR transcripts, but no pre-Tα chain (K. Suzuki et al., Immunity 13, 691 (2000) or RAG-2 (D. Guy-Grand et al., J Exp Med 197, 333 (2003)). It has been demonstrated herein that, indeed, intestinal αβ and γδ T cells are not derived from intestinal RORγt⁺ cells, which include the lin⁻c-kit⁺IL-7Rα⁺ CP cells. Although it may be concluded that intestinal αβ T cells are derived from DP thymocytes, the cell fate mapping experiments do not exclude a CP-independent extrathymic origin of γδ IEL (T. Lin et al., Eur J Immunol 24, 1080 (1994)), since these cells are not derived from RORγt⁺ cells. Finally, the earlier finding that αβ IEL are present in athymic mice does not contradict our conclusions. The presence of these IEL is accompanied by the appearance of RAG⁺ DP T cells in mLNs, but such cells are absent in euthymic mice (D. Guy-Grand et al., J Exp Med 197, 333 (2003)). Extrathymic T cell development thus appears to be a de novo pathway in lymphopenic mice, such as athymic or neonataly thymectomized mice.

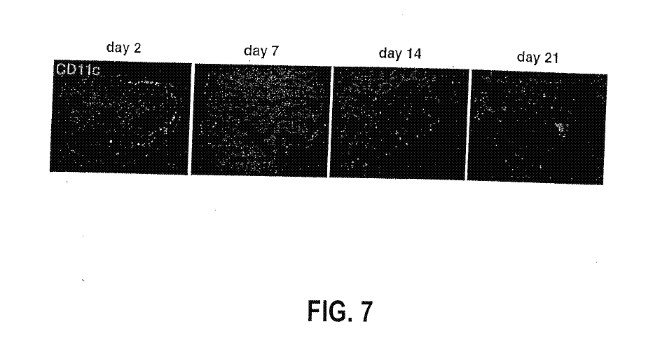

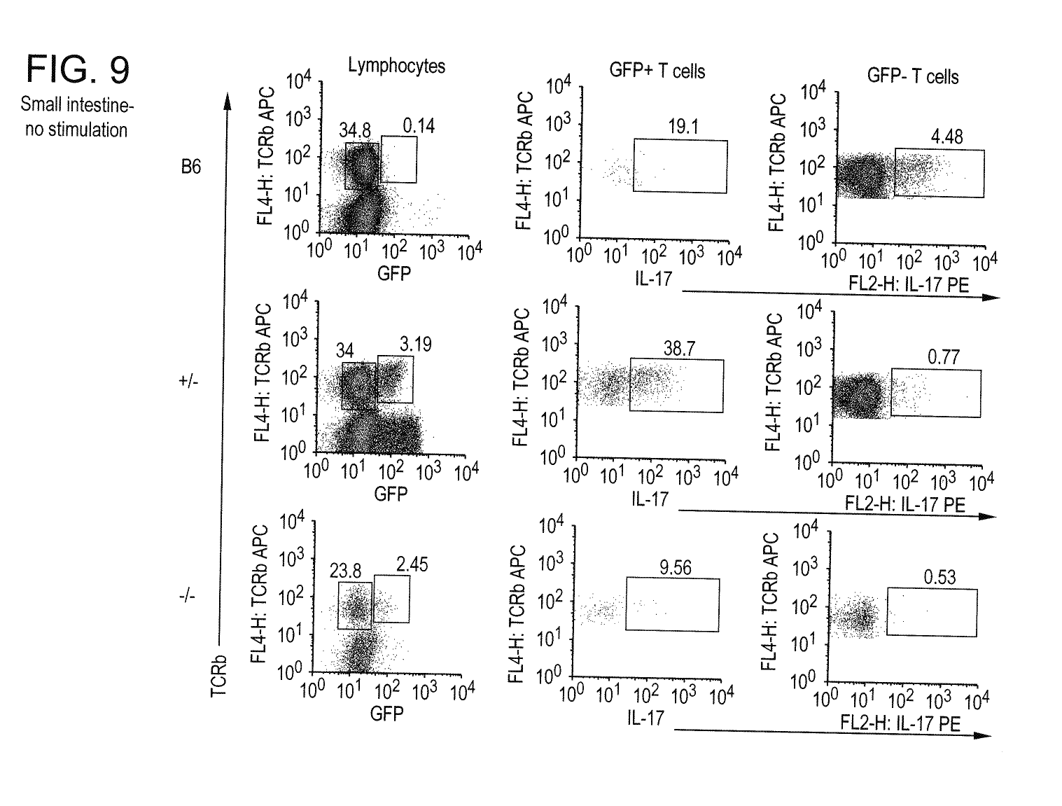

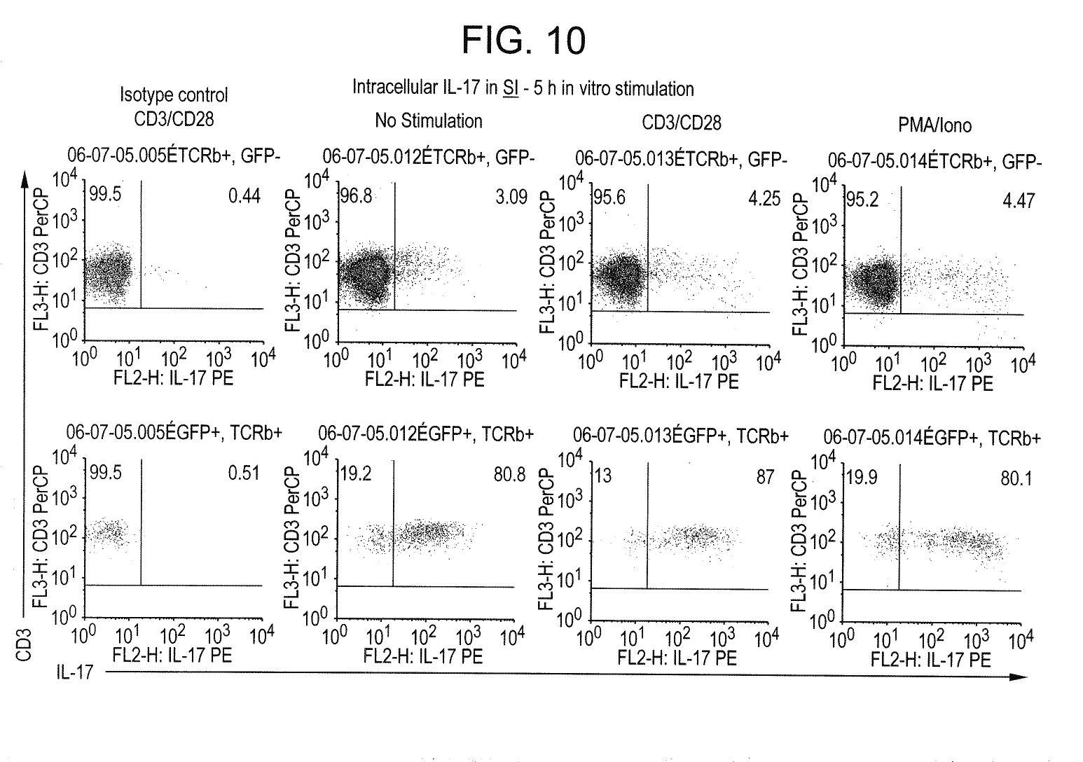

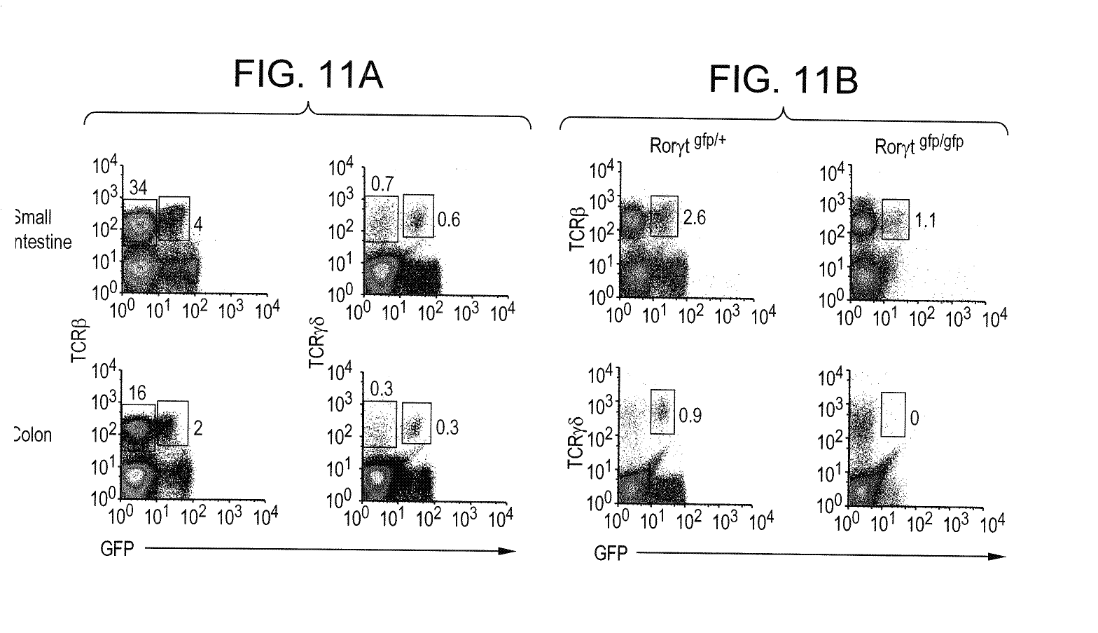

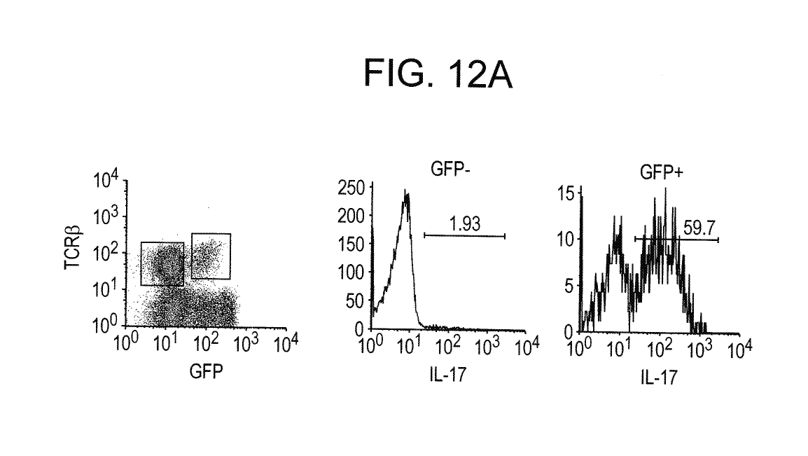

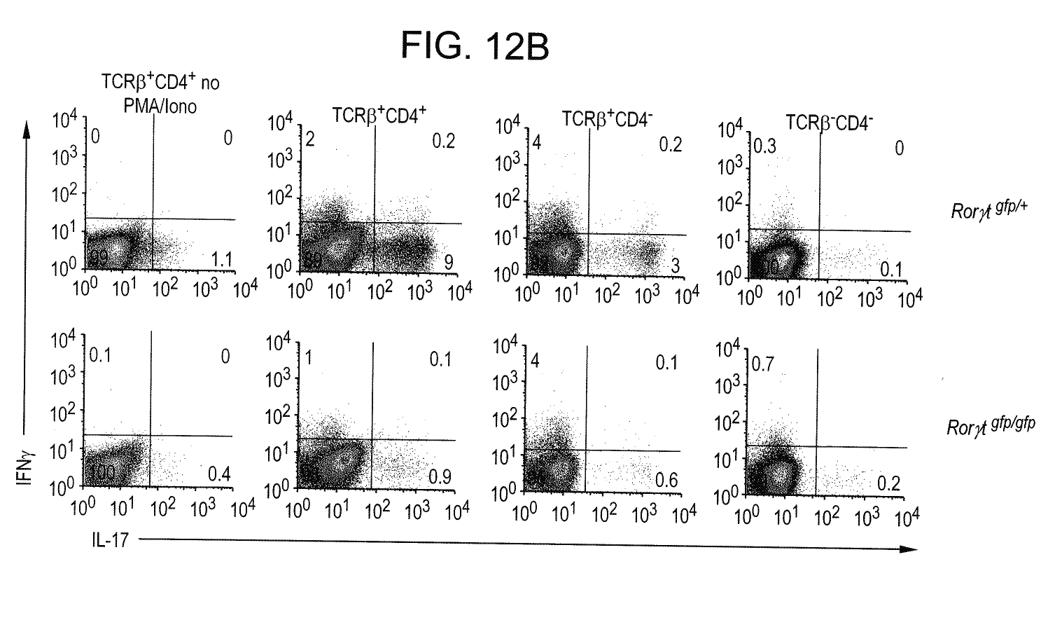

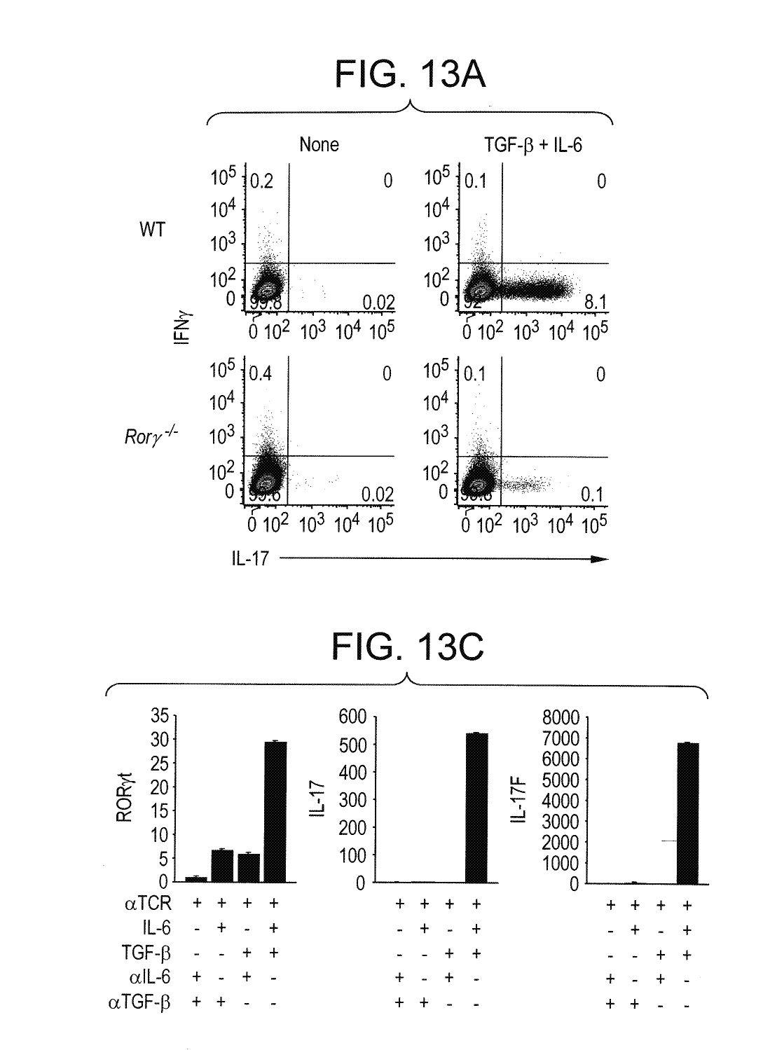

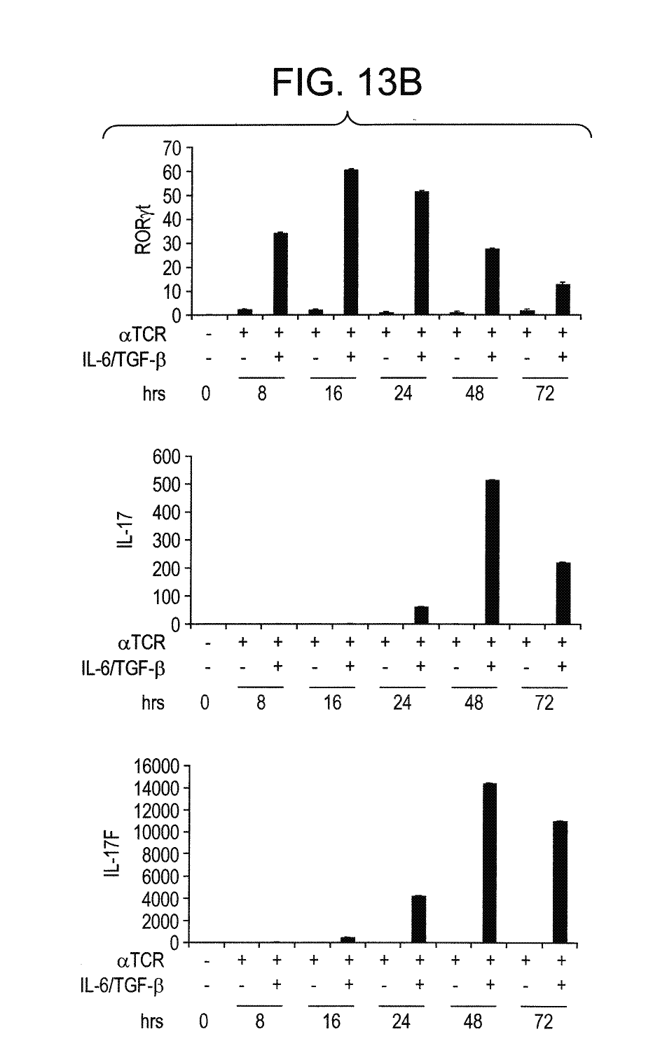

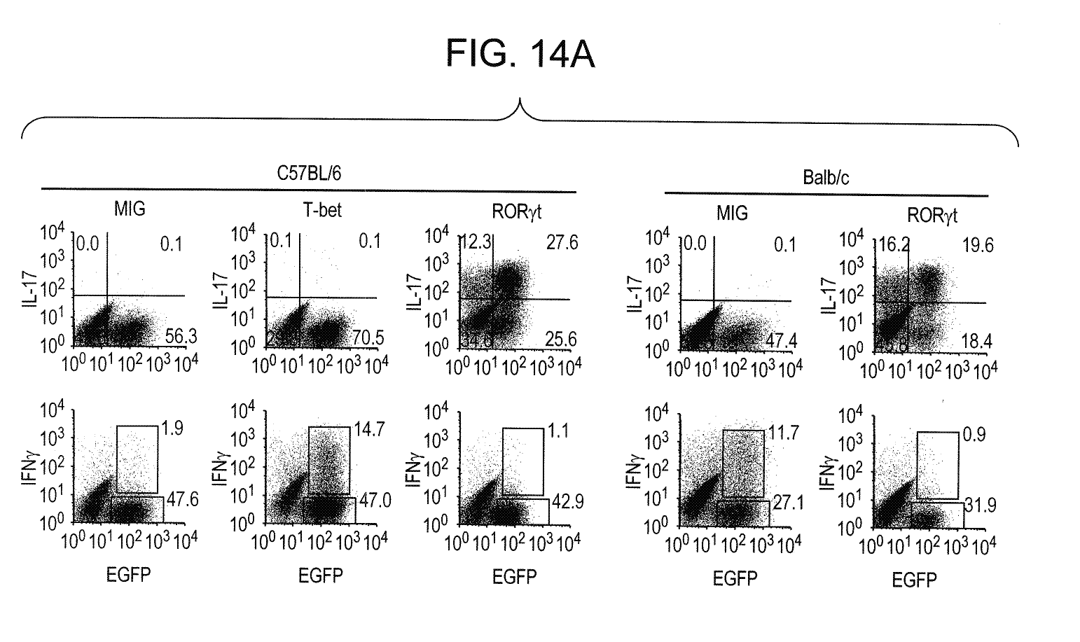

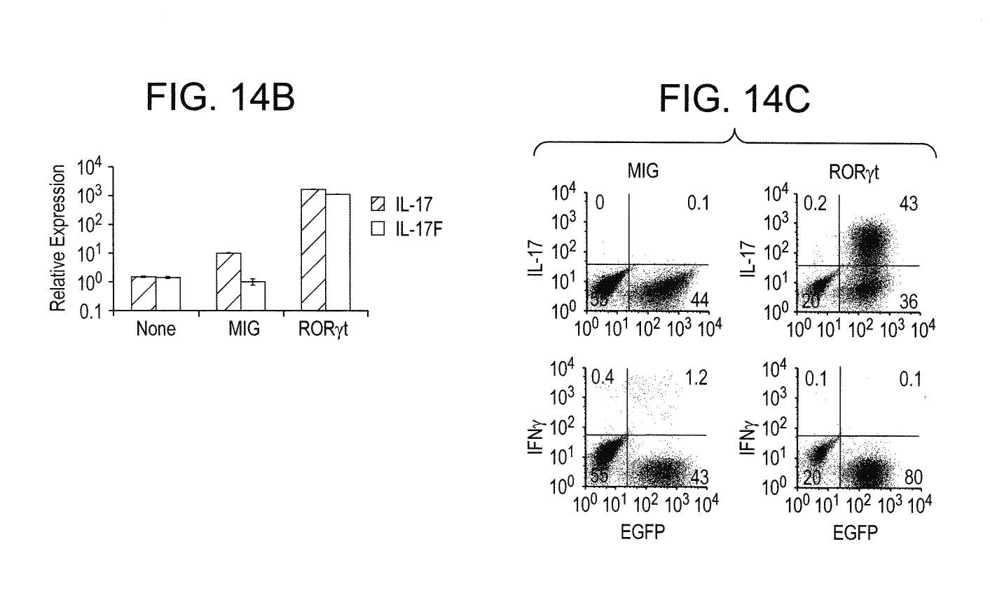

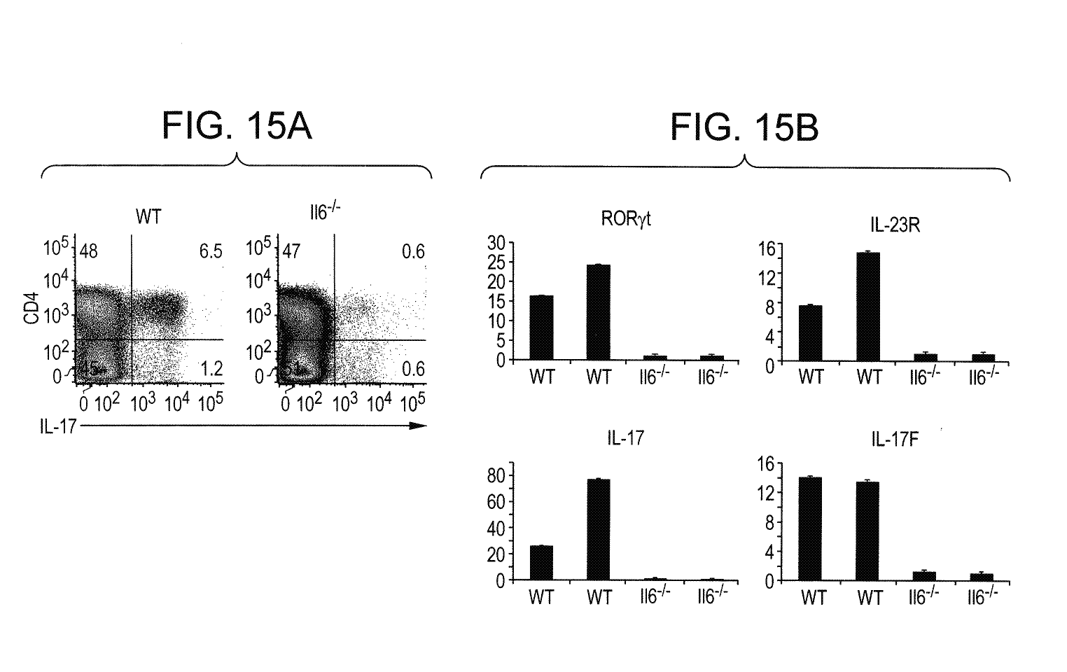

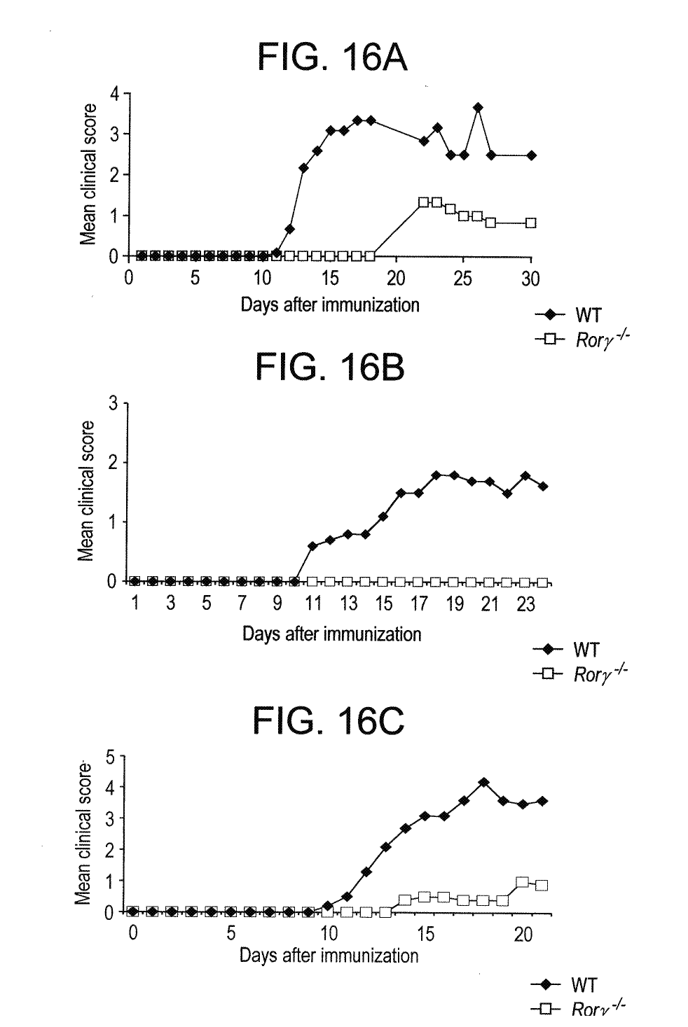

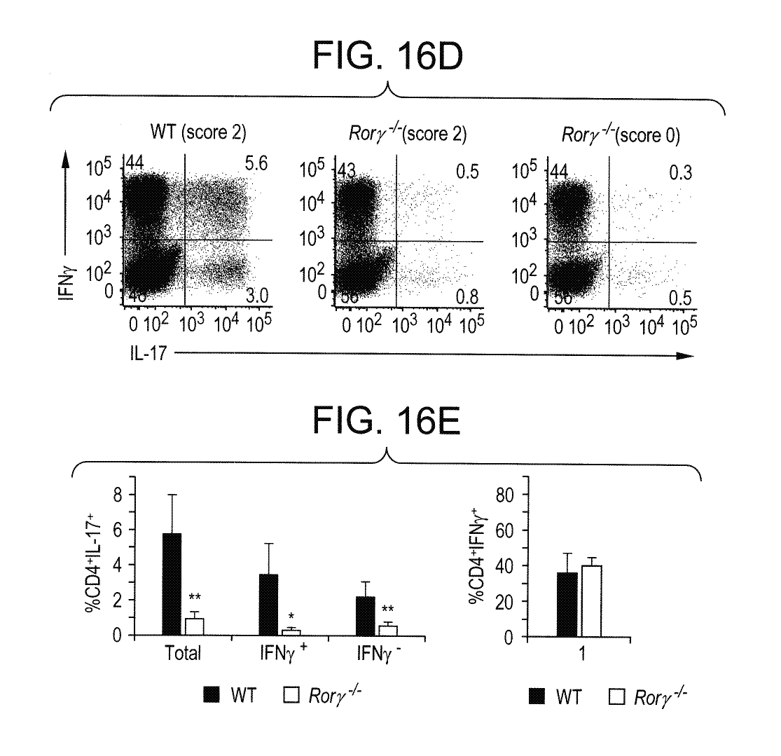

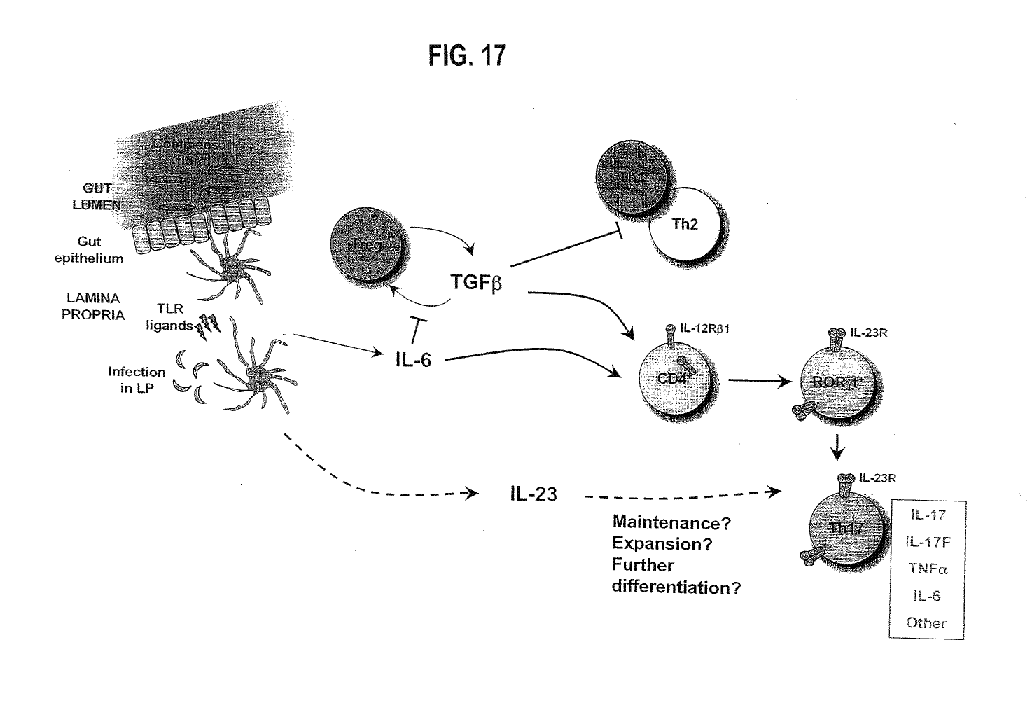

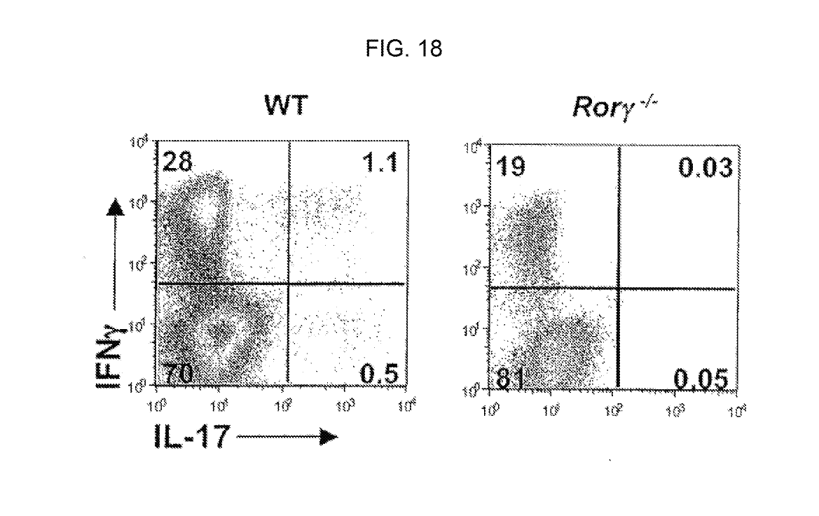

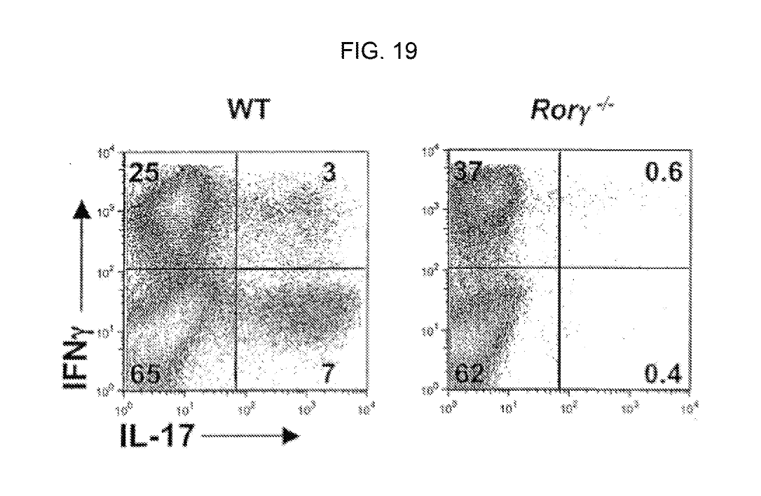

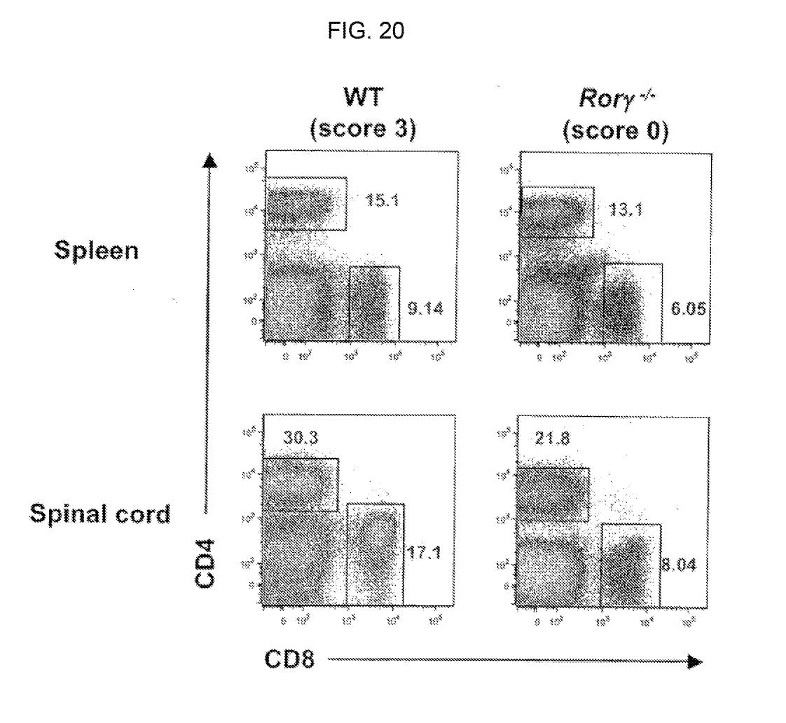

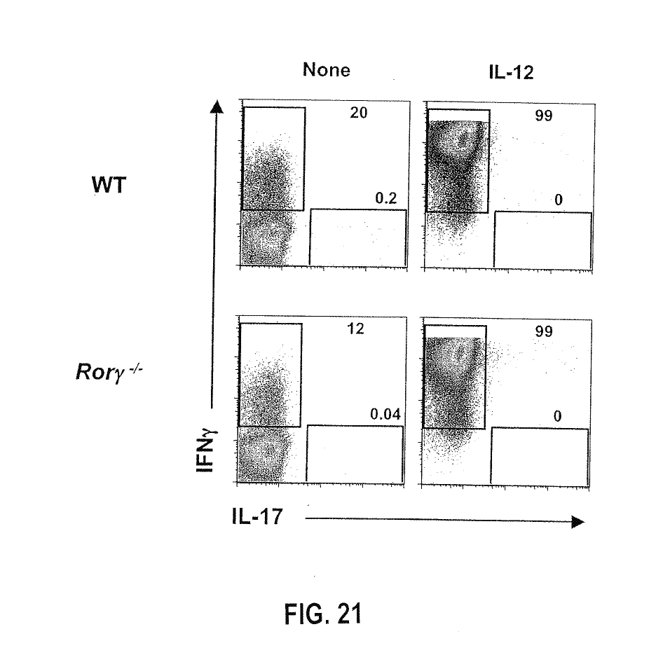

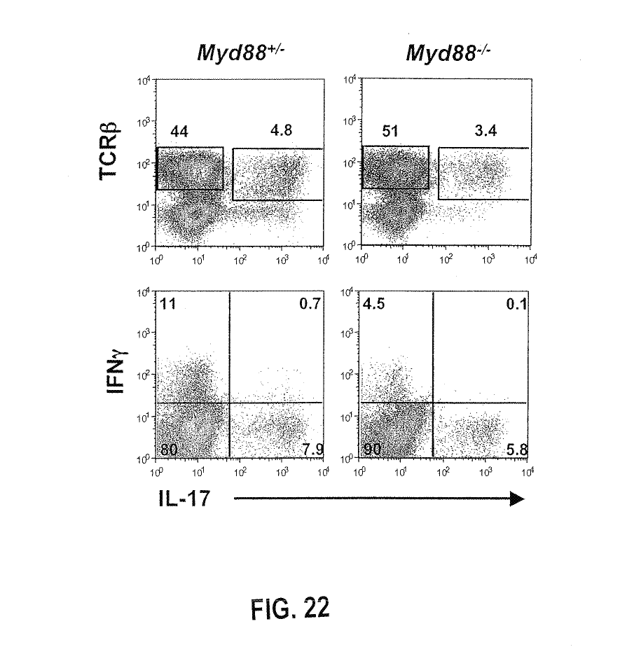

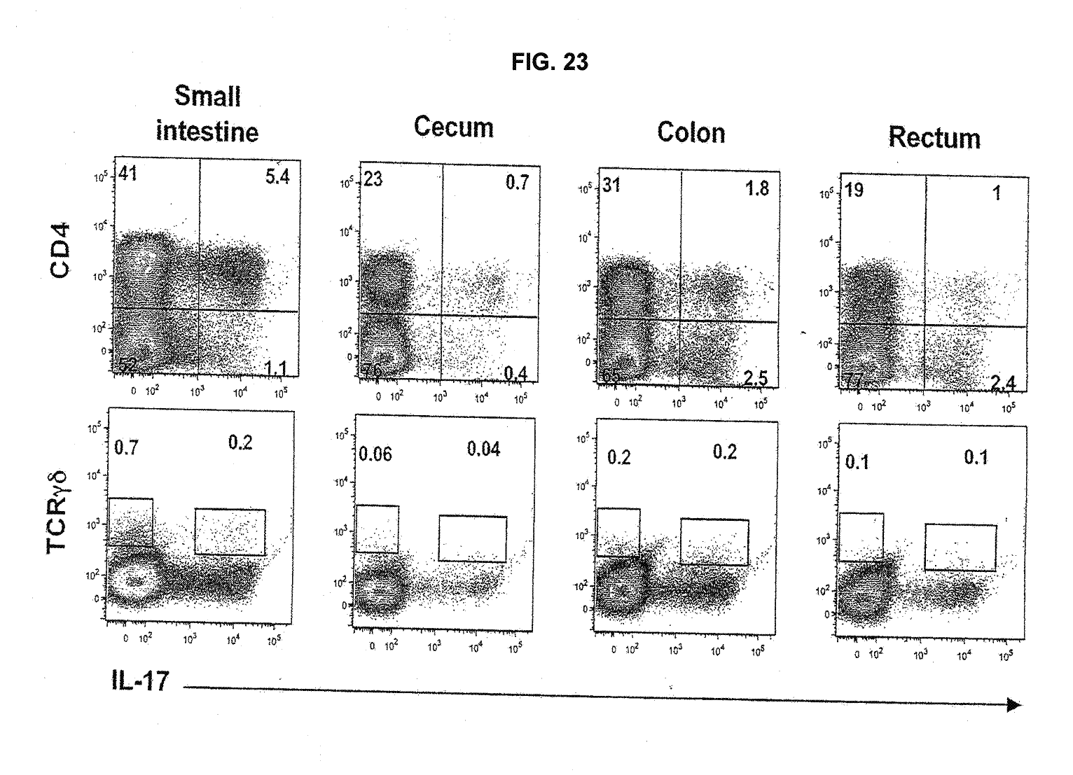

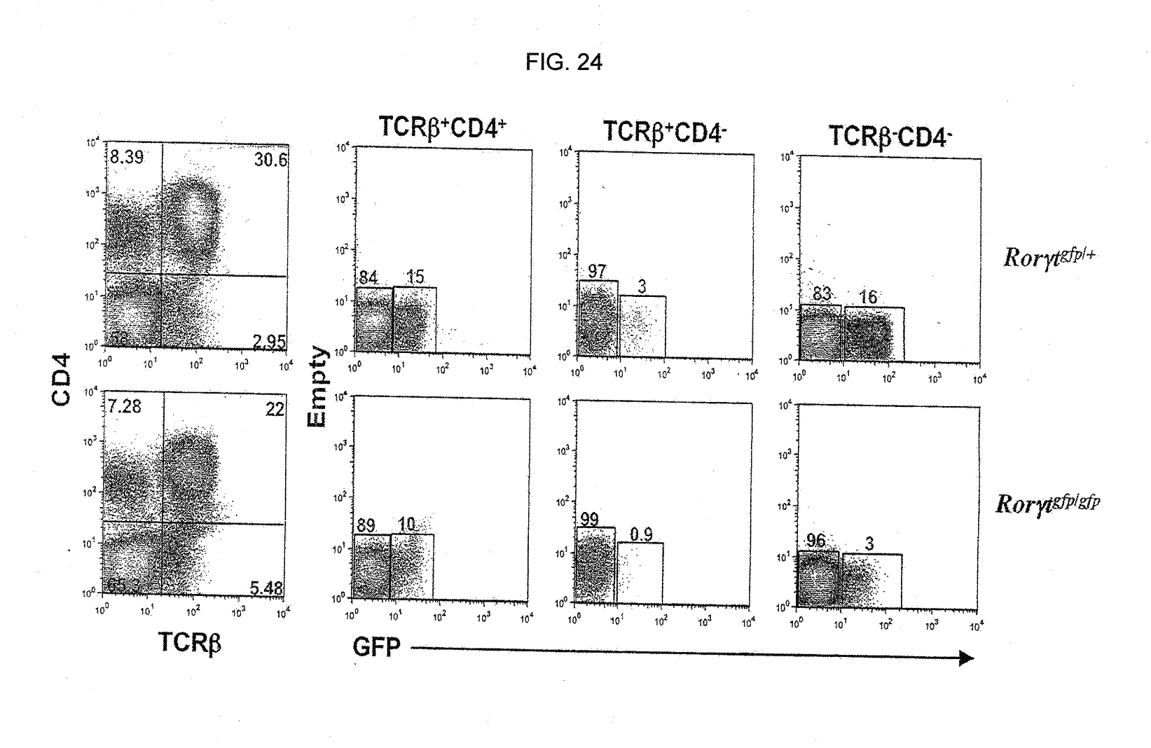

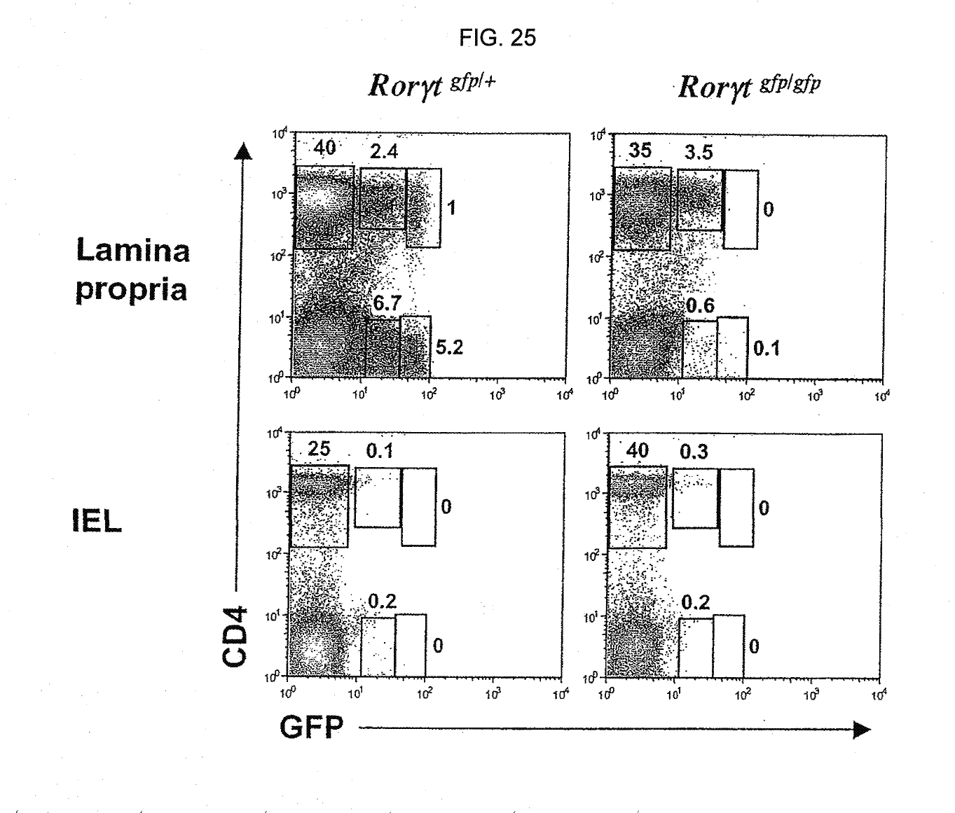

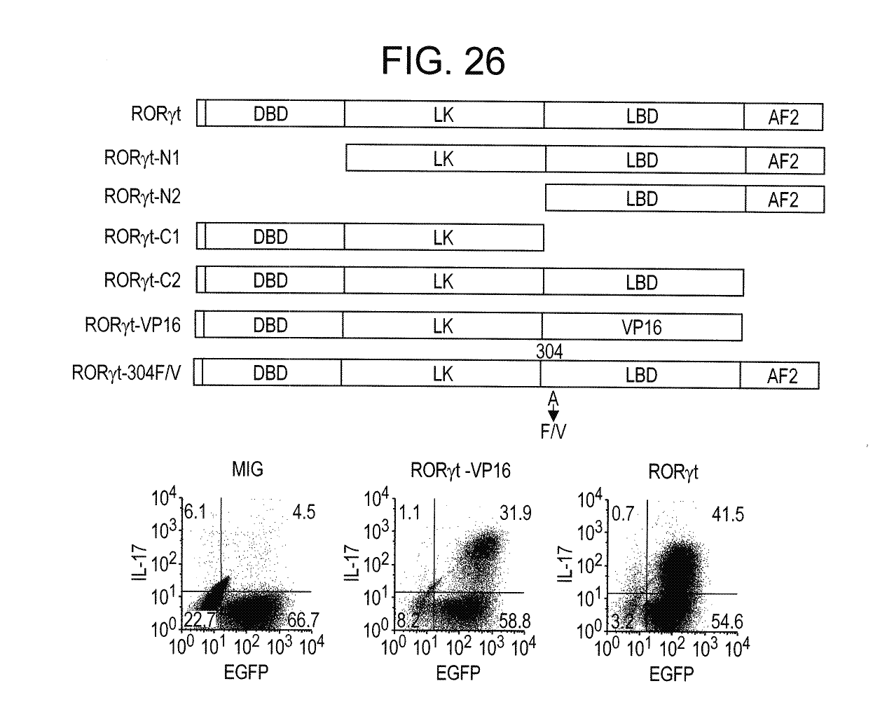

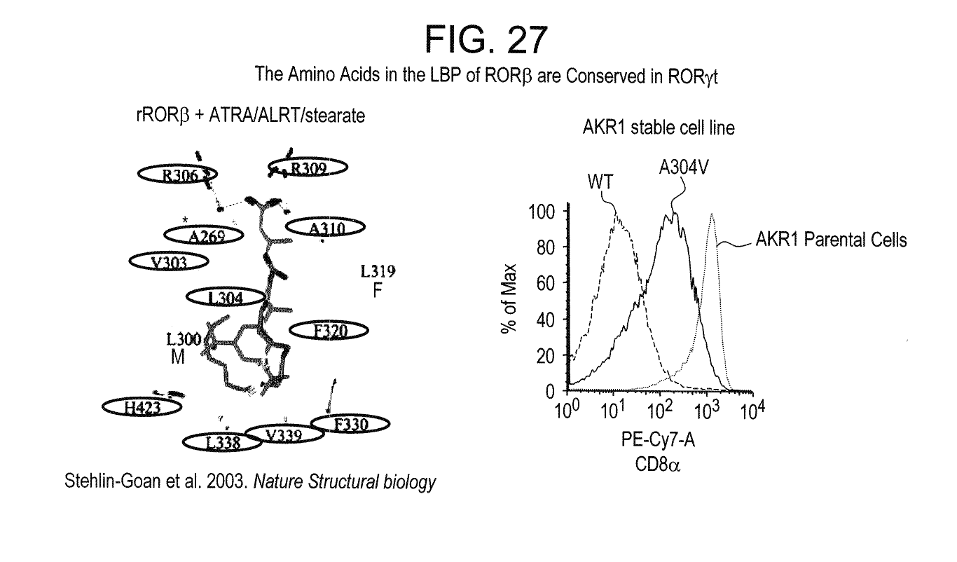

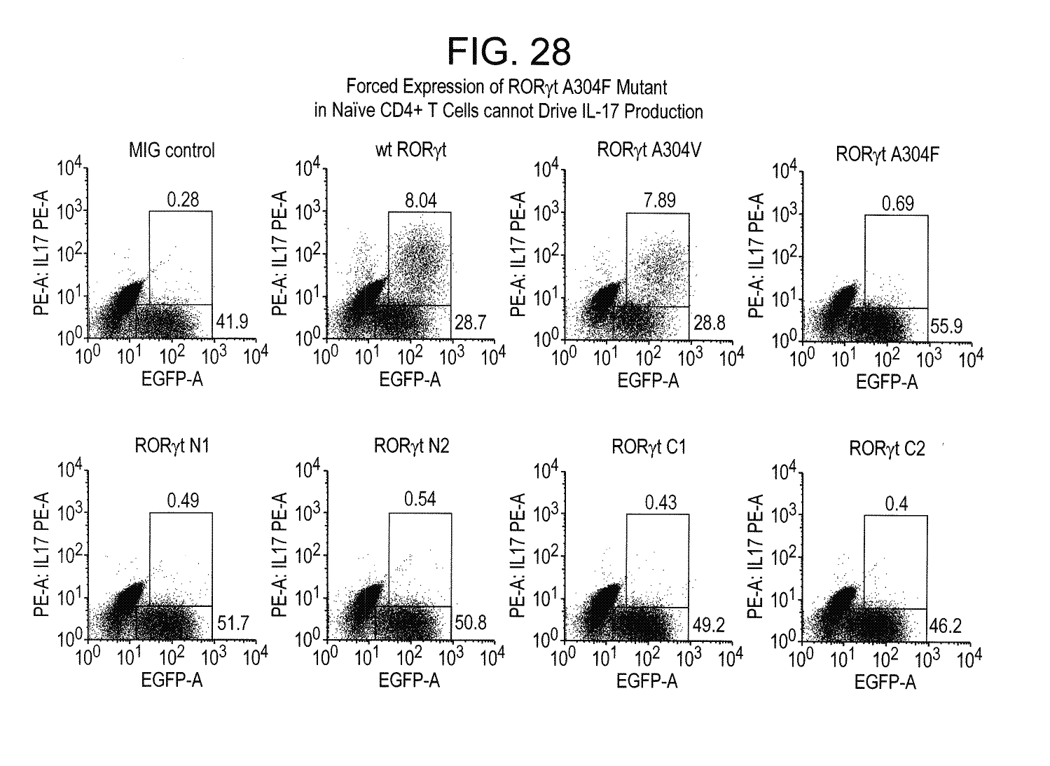

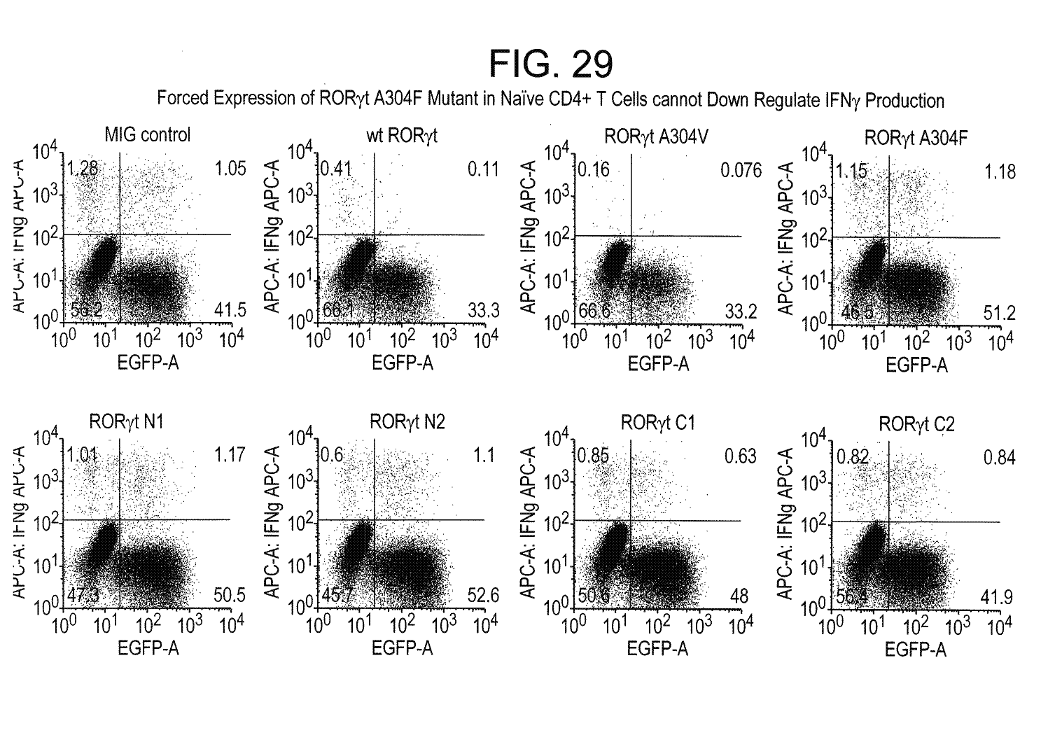

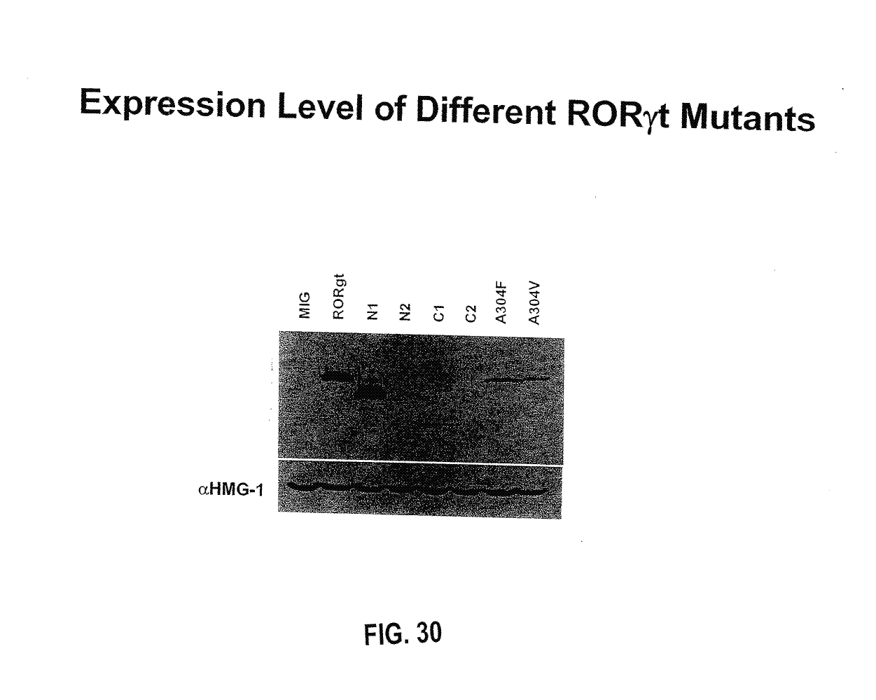

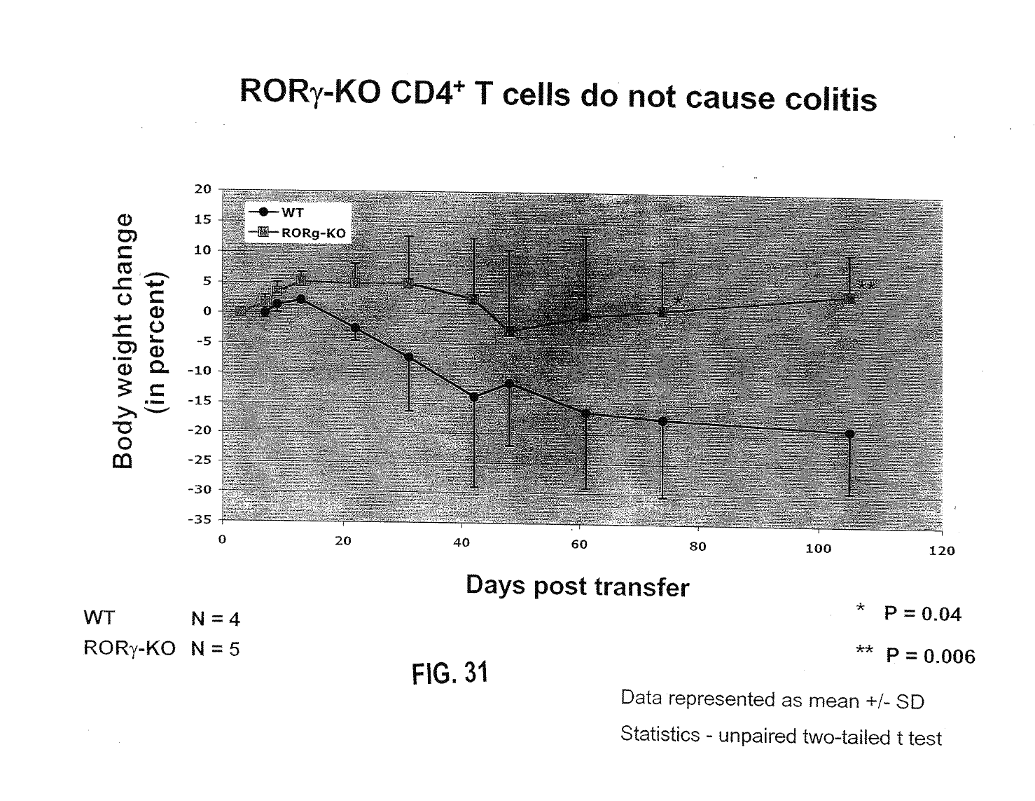

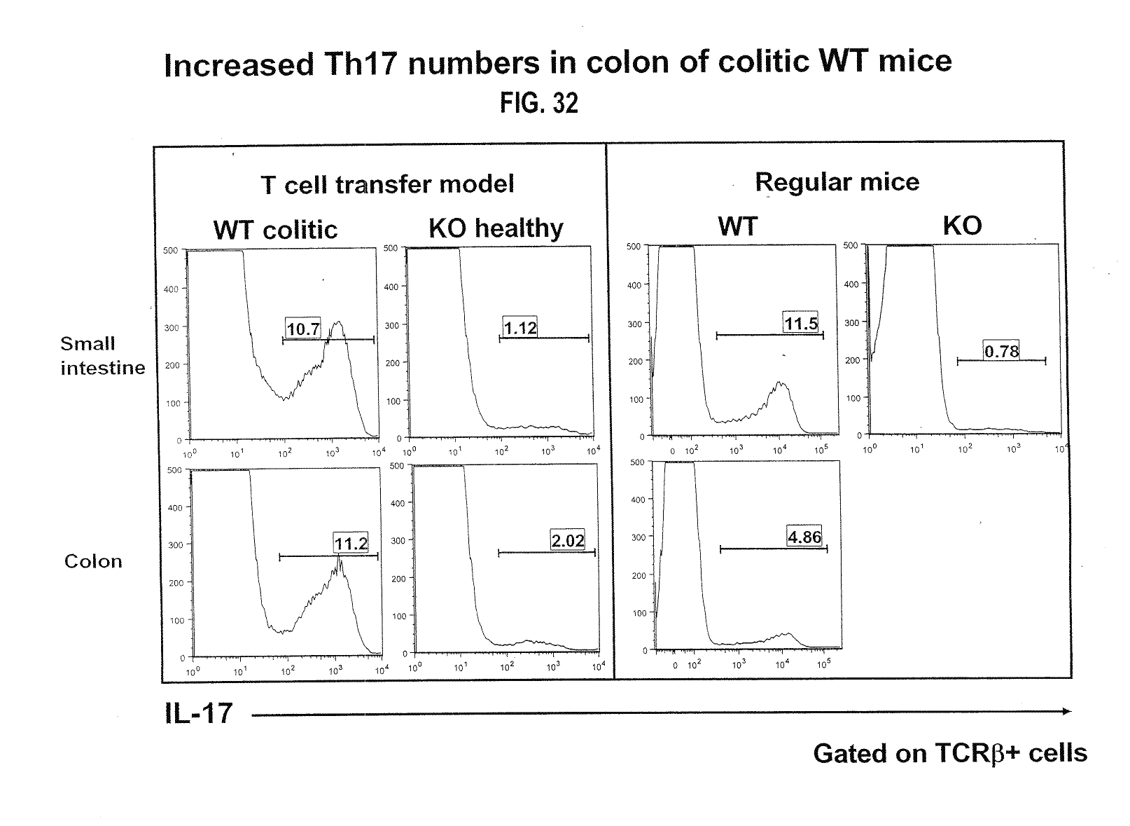

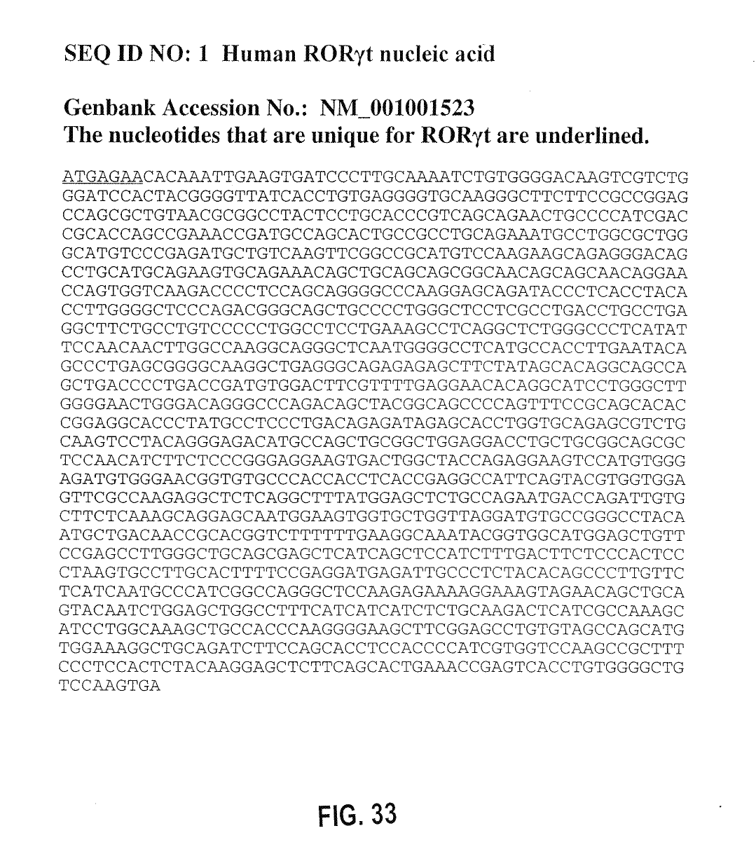

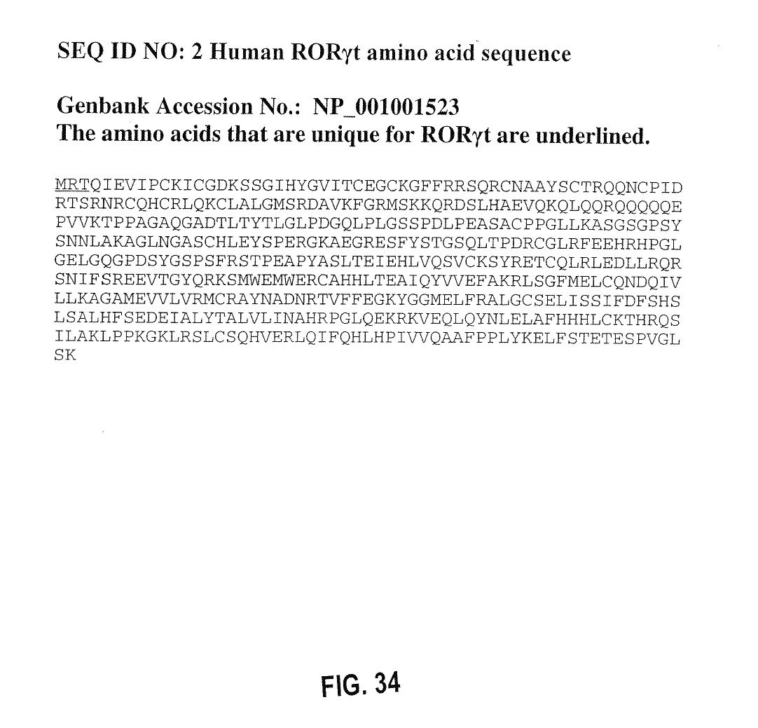

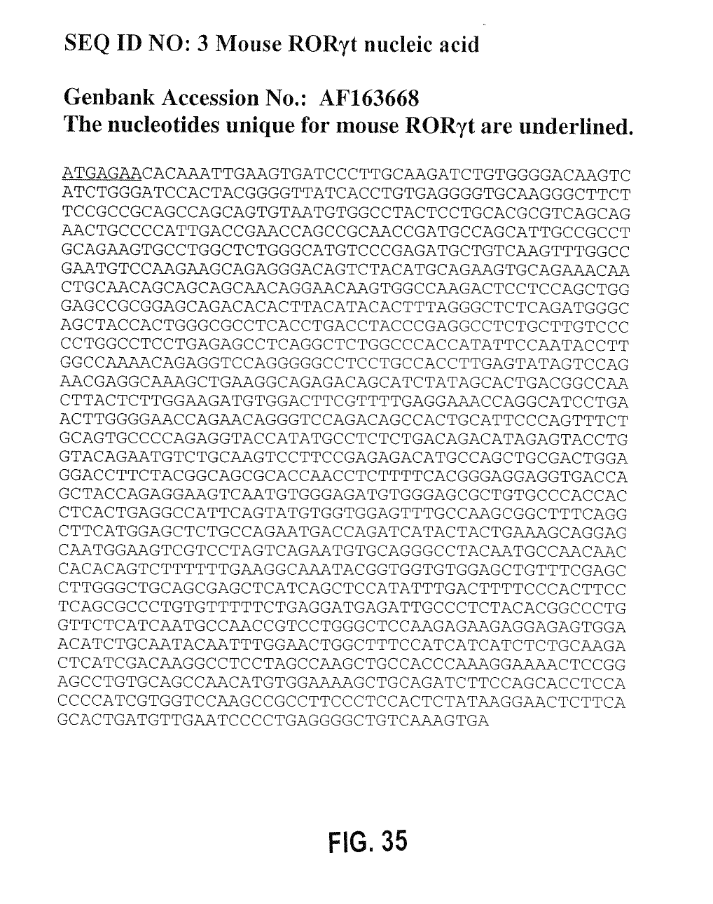

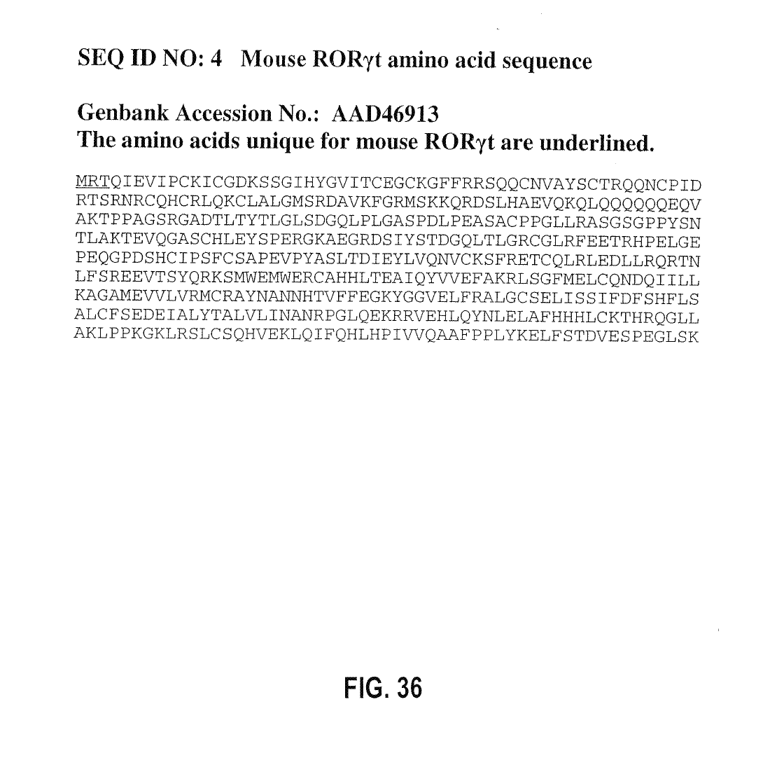

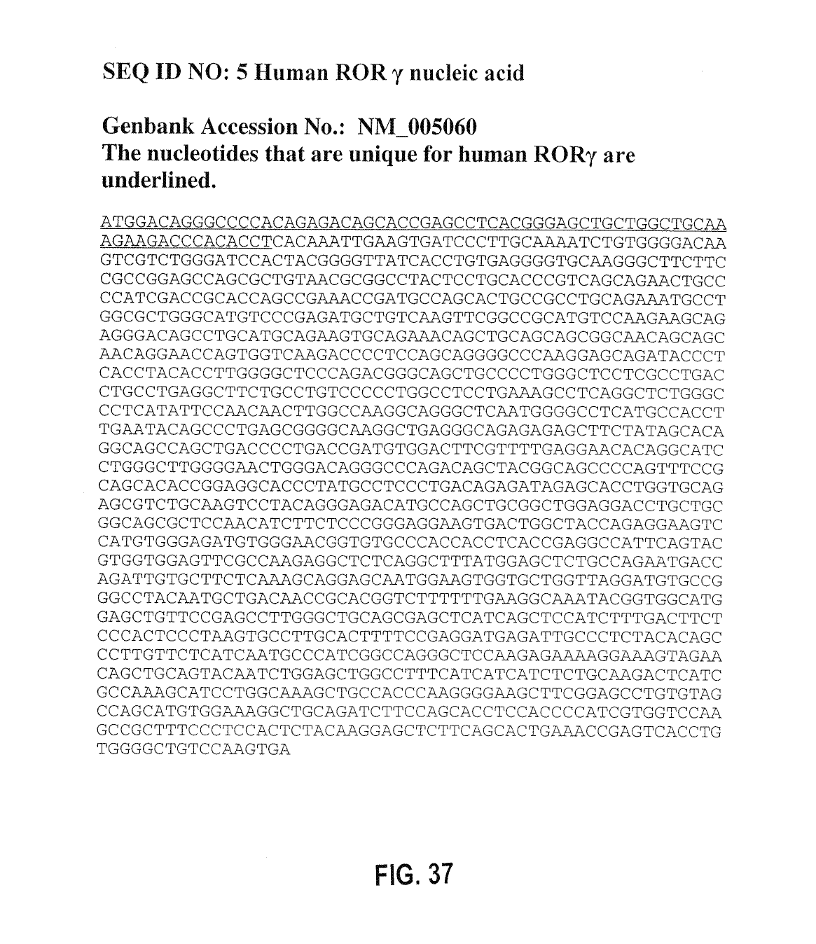

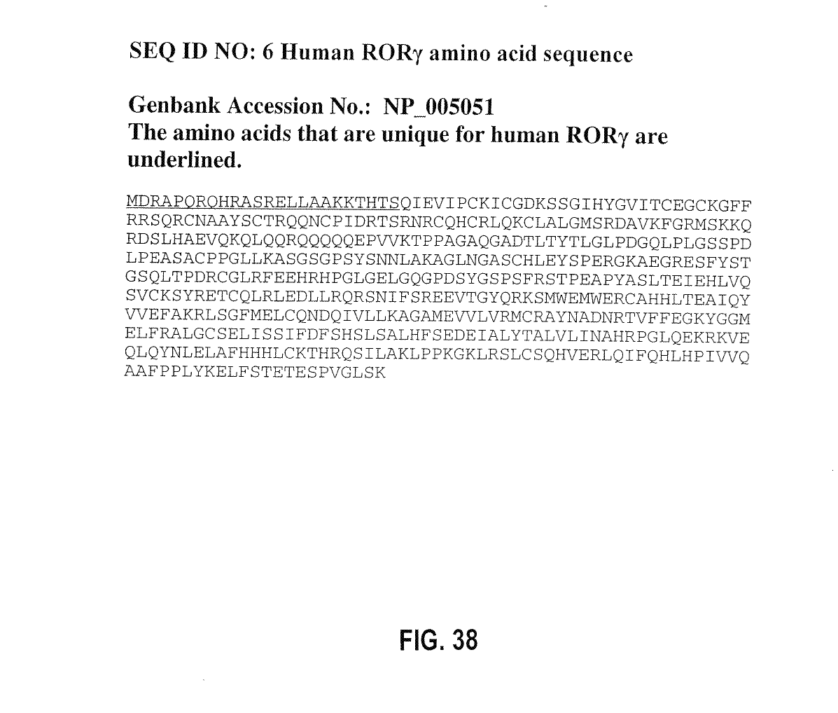

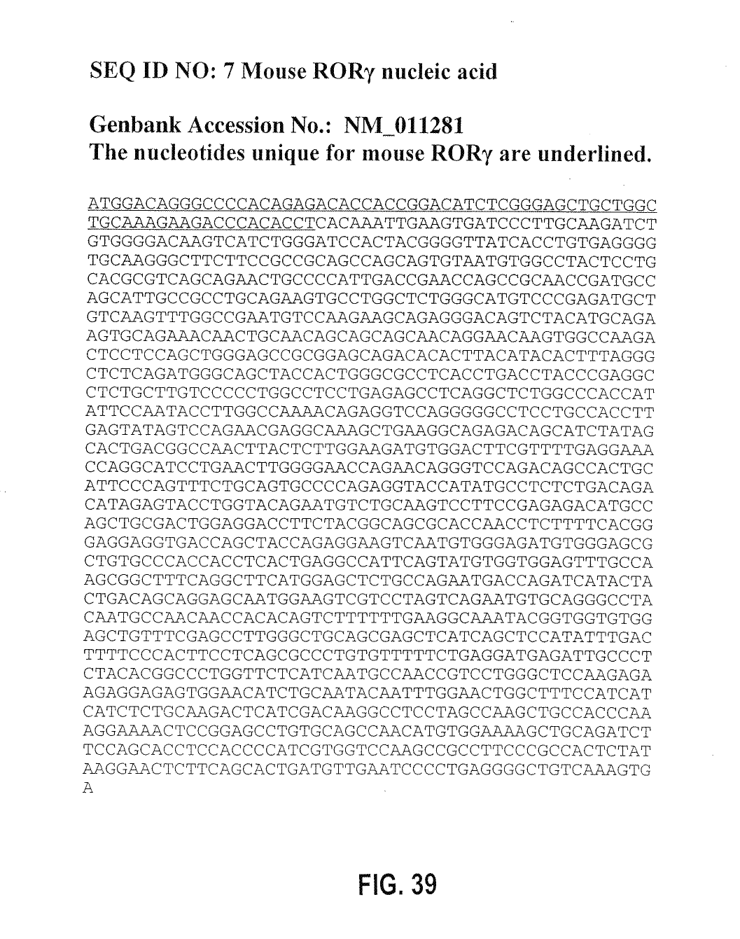

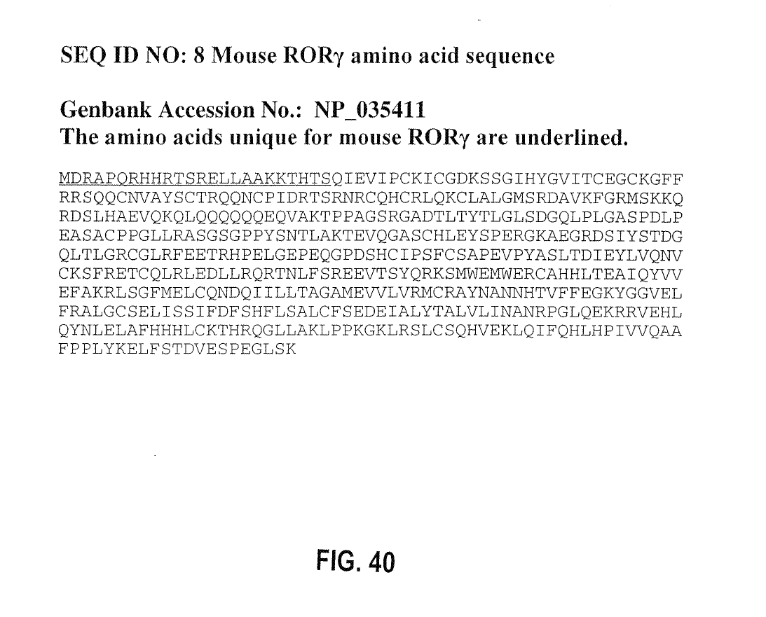

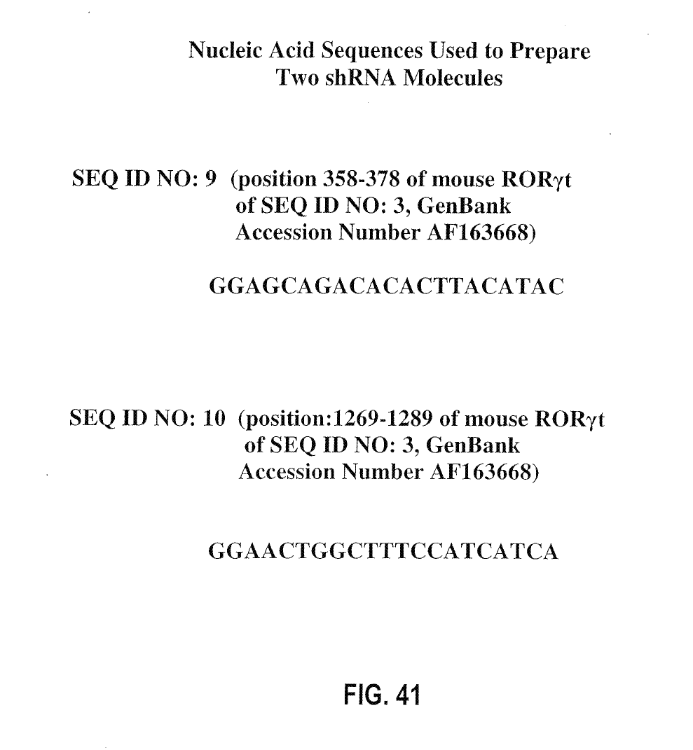

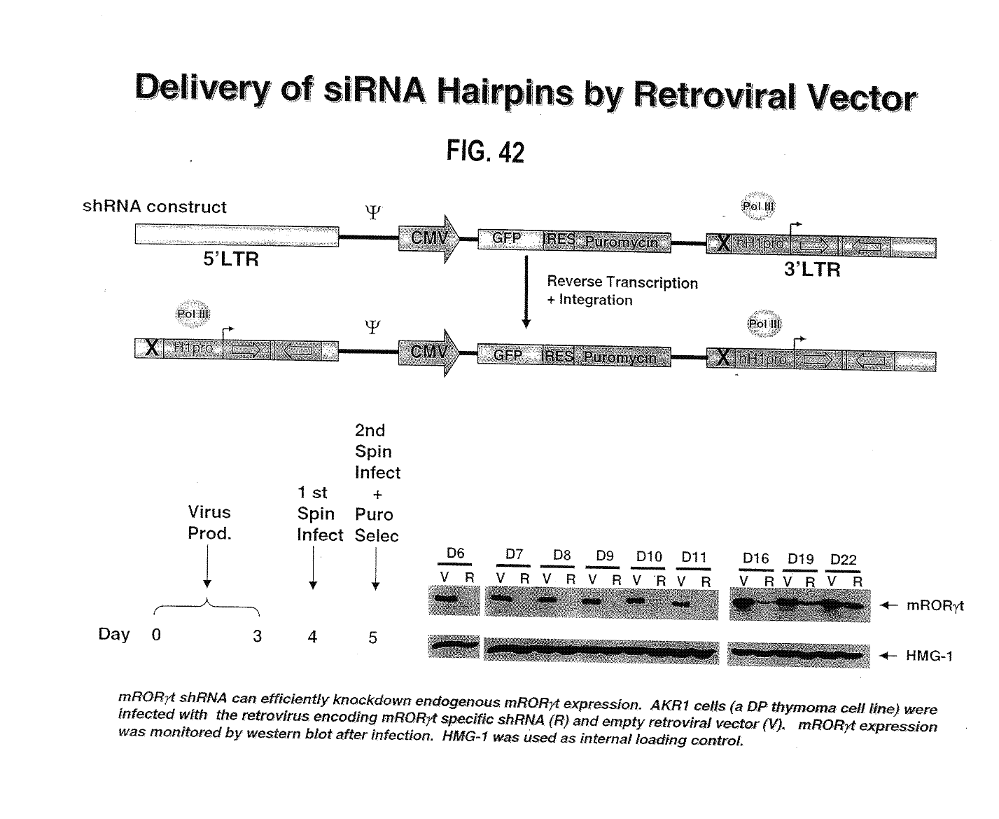

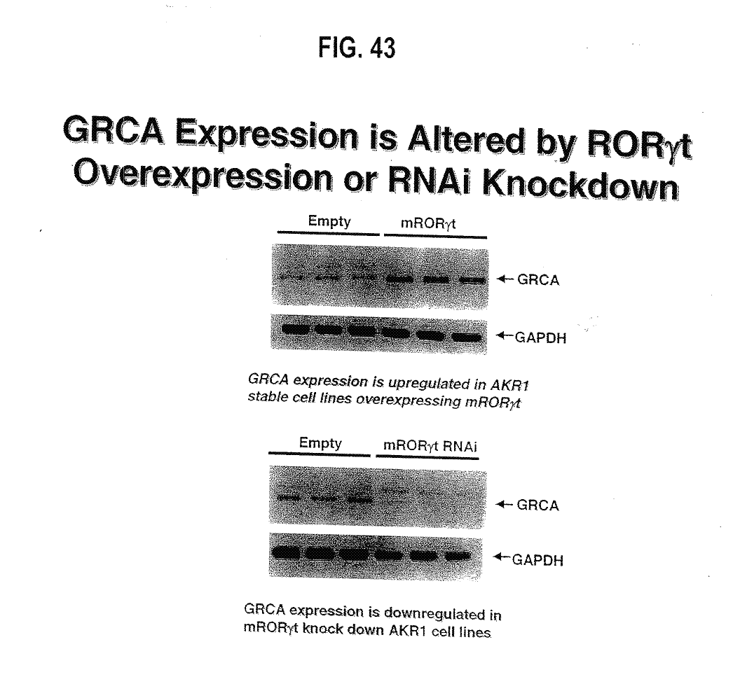

Adult intestinal RORγt⁺ cells share all developmental, phenotypic, and functional features with fetal RORγt⁺ LTi cells (Table S2). Both cell (G. Eberl et al., Nat Immunol 5, 64 (2004); R. E. Mebius, P. Rennert, I. L. Weissman, Immunity 7, 493 (1997)) types require RORγt and the inhibitor of bHLH transcription factors Id2 for their development (data not shown). Furthermore, in LTα-deficient mice, LTi cells develop but do not activate mesenchymal cells and fail to induce further LN and PP development (G. Eberl et al., Nat Immunol 5, 64 (2004)). Similarly, intestinal RORγt⁺ cells are present in LTα-deficient mice, but fail to cluster into mature CPs (FIG. 6). Together, these data suggest that intestinal RORγt⁺ cells are the adult equivalent of fetal LTi cells. In accordance with this hypothesis, the data presented herein show that intestinal RORgt⁺ cells are required for the development of CPs and ILFs in the adult intestine. The relationship between fetal LTi, the small CPs and the more elaborate ILFs will be important to elucidate. Although RORγt⁺ cells are continuously present in the intestinal lamina propria from the fetus to adulthood (FIG. 7), it is unclear if they represent LTi cells that persist post-natally. It has been reported that fetal or neonatal cells with the surface phenotype of LTi cells can develop in vitro into NK cells and antigen presenting cells (APCs) (R. E. Mebius et al., J Immunol 166, 6593 (2001); H. Yoshida et al., J Immunol 167, 2511 (2001)). This is not the case in vivo, since the progeny of RORγt⁺ cells do not include NK cells, macrophages or dendritic cells (FIGS. 3B and 5D). Because the progeny of extrathymic RORgt⁺ cells cannot be found in the intestine or in lymphoid organs, we propose that these cells serve as organizers of lymphoid tissues, both in fetal LN and PP development and in adult CP and ILF development. Furthermore, as noted in FIG. 8, we determined the presence of a subpopulation of T cells in the small and large intestine in the RORγtKI (knockin) mice. We tested these GFP+ T cells to determine whether they produced IL-17. As shown in FIG. 9, CD3 T cells were present that produced IL-17 in the small intestine, not the large intestine. Thus, RORgt+ cells in the small intestine may be proinflammatory and induce colitis under certain conditions. Thus, elimination of RORgt+ cells ThIL-17 cells in the intestine may be beneficial for intestinal inflammation. However, none of the T cells in the large intestine produces IL-17 (FIG. 10).

In germ-free mice, ILFs are small and harbor a majority of CP-like lin⁻c-kit ⁺ cells (H. Hamada et al., J Immunol 168, 57 (2002)). Moreover, the number of ILFs is increased in dextran sulfate-induced colitis in mice (T. W. Spahn et al., Am J Pathol 161, 2273 (2002)), as well as in Crohn's disease (E. Kaiserling, Lymphology 34, 22 (2001)) and ulcerative colitis in humans (M. M. Yeung et al., Gut 47, 215 (2000)). We therefore propose that CPs develop into ILFs in the adult intestine following inflammatory innate immune signals transmitted to the RORγt⁺ cells. RORγt⁺ may thus be an attractive therapeutic target for inflammatory bowel diseases, as well as other inflammatory or autoimmune diseases or conditions.

TABLE S1
The progeny of RORγt+ cells and CD4+ cells

| | | | | | | | | | | | Intestine | | | |
|---|---|---|---|---|---|---|---|---|---|---|---|---|---|---|
| | Thymus | | | | Spleen | | | | | | | Tgd | Tab | |
| ckit⁺ | DN | DP | SP4 | SP8 | B⁻ | T⁻ | B | T4 | T8 | Total | 8⁻ | 8ab | 8aa | IL-7R⁺ |
| RORγt-EGFP | − | + | +/−¹ | +/−¹ | − | − | − | − | − | − | − | − | − | + |
| RORγt-Cre | − | + | + | + | − | − | + | + | − | + | + | + | + | + |
| TG/R26R | | | | | | | | | | | | | | |
| CD4-Cre^{TG}/ | − | + | + | + | − | − | + | + | − | + | + | + | + | − |
| R26R | | | | | | | | | | | | | | |

¹Low levels of EGFP were also detected in CD4+ and CD8+ single positive (SP) thymocytes, even though Rorc(γt) mRNA and protein was not detected in these population. This may be due to the long half-life of EGFP (>24 hrs), present in SP thymocytes even after cessation of Rorc(γt) transcription.

TABLE S2
Phenotypic and developmental similarity of fetal RORγt + LTi cells and
adult intestinal RORγt + cells.

| | Fetal LTi cells | Intestinal RORγt + cells |
|---|---|---|
| Phenotype | | |
| RORγt | + | + |
| IL-7Ra | + | + |
| c-kit | +¹ | + |
| CD44 | + | + |
| CD45 | + | + |
| ICAM-1 | + | + |
| CD4 | +/−² | +/− |
| CD3 | − | − |
| TCRαβ | − | − |
| TCRγδ | − | − |
| B220 | − | − |
| CD11b | − | − |
| CD11c | − | − |
| NK1.1 | − | − |
| DX5 | − | − |
| Gr-1 | − | − |
| Gene dependence | | |
| RORγt | + | + |
| Id2 | + | + |
| LTα | −³ | − |
| RAG-2 | − | − |

¹c-kit is expressed by CD3 − IL-7Ra + cells in PP anlagen and in low amounts by CD3 − CD4 + cells in newborn mesenteric LNs.
²CD4 is expressed by 50% of LTi cells and by 30-40% of intestinal RORγt + cells.
³In LTa-deficient mice, LTi cells are present in LN and PP anlagen, but do not induce activation of mesenchyma; RORγt + cells are present in the adult intestine, but do not cluster into mature cryptopatches.

### Example 2

### In Vivo Assessment of Modulators of RORγt in Inflammatory Bowel Disease

### Materials and Methods

### Ulcerative Colitis Model

Ulcerative colitis is induced in Sprague Dawley rats (7-8 weeks old) by anal administration of a solution in which 90 mg of trinitrobenzenesulfonic acid (TNB) is dissolved in 1.5 ml. of 20% ethanol. Certain groups of rats are treated with various doses of the RORγt modulator and other groups are treated with a vehicle control. In these studies, the preferred route of administration of the RORγt modulator is by catheter to deliver the compound directly to the colon. Most preferably, a rubber catheter such as a Nelaton catheter No. 8 is used (Rush Company, West Germany). The compound is preferably introduced about 6 cm from the rectum in the rat. One of skill in the art will be familiar with the use of such catheters to deliver compounds to the desired site in rats of varying ages and weights and in other experimental animals. During the experiments rats are clinically evaluated daily, and presence or absence of diarrhea is monitored.

At one to two weeks after induction of colitis, the rats are sacrificed by decapitation and evaluated for severity of colonic lesions and general colonic pathology to evaluate the development of ulcerative colitis. The colon is rapidly removed, opened, rinsed in saline, blotted gently, weighed and fixed in 10% formalin. Standardized sections of ileum, jejunum, duodenum, stomach, liver, pancreas, kidneys and lungs are also fixed, and processed for histologic examination. Additional sections from grossly involved and uninvolved areas of colon, ileum and jejunum are frozen and subsequently homogenized for the determination of colonic myeloperoxidase activity by the method of Bradley et al. (Bradley, P. P., et al., J. Invest. Dermatol. 78:206-209 (1982)) using 0.0005% hydrogen peroxide as a substrate. This enzyme, located mainly in the azurophilic granules of polymorphonuclear leukocytes is used as a quantitative index of inflammation (Morris, G. P., et al., Gastroenterology 96:795-803 (1989); Bradley, P. P., et al., J. Invest. Dermatol. 78:206-209 (1982); Krawisz, J. E., et al., Gastroenterology 47:1344-1350 (1985)).

For morphologic studies at the light microscopy level 2-4 mm long tissue sections of tissue are fixed in 10% buffered (pH7) formalin, dehydrated and embedded in paraffin or in the J8-4 plastic embedding medium. Sections (1-5 um) from all organs are stained with hematoxylin and eosin (H&E) and, in addition, sections from stomach and duodenum are also stained with the periodic acid-Schiff (PAS) technique.

Morphometric analysis of colonic lesions is performed by stereomicroscopic planimetry (Szabo, S., et al., J. Pharm. Methods 13:59-66 (1985); Szabo, S., et al., Gastroenterology 88:228-236 (1985); Szabo, S., et al., Scand. J. Gastroenterol. 21 Suppl.:92-96 (1986)). In addition, “damage scores” 0-5 are calculated using a combination of gross and histologic assessment of the extent of TNB-induced colonic lesions (Morris, G. P., et al., Gastroenterology 96:795-803 (1989)). Thus, there are four quantitative endpoints in evaluating the experimental colonic lesions: planimetry (mm²) of involved colon, damaged score (grades 0-5) derived from gross and histologic evaluation, colon weight (Calkins, B. M., et al., Epidemiol. Rev. 8:60-85 (1986)) indicating edema, inflammatory infiltrate and tissue proliferation, as well as myeloperoxidase activity quantitatively reflecting the intensity of inflammation.

All the four endpoints have been found sensitive and quantitive indicators of the severity and extent of induced experimental gastric and colonic lesions (Szabo, S., et al., Gastroenterology 86:1271 (1984); Szabo, S., et al., Dig. Dis. Sci. 34:1323 (1989); Szabo, S., et al., J. Pharm. Methods 13:59-66 (1985); Morrison, B. C., et al., eds., Gastrointenstinal Pathology, 2d ed., London (1979); Szabo, S., et al., Scand. J. Gastroenterol. 21 Suppl.:92-96 (1986)).

For further characterization of chronic inflammation, standard immunoperoxidase and cytochemical methods are used to selectively obtain and count subpopulations of B and T-lymphocytes in the inflamed colon. The colons of rats which receive the vascular tracer monastral blue for the detection of early vascular injury, which is well established in the pathogenesis of chemically induced gastric lesions (Szabo, S., et al., Gastroenterology 88:228-236 (1985); Szabo, S., et al., Scand. J. Gastroenterol. 21 Suppl.:92-96 (1986)), are cleared in glycerol for 24 hr after planimetric assessment of mucosal ulcers. The area of blood vessels labeled with deposition of monastral blue between the damaged endothelium and vascular basement membrane, are measured by stereomicroscopic planimetry (Szabo, S., et al., Gastroenterology 88:228-236 (1985); Szabo, S., et al., Scand. J. Gastroenterol. 21 Suppl.:92-96 (1986)).

Tissue samples from colon and ileum from rats killed up to 2 days after IA or NEM are fixed in Karnovsky's fixative for electron microscopy, dehydrated in graded ethanol, embedded, cut and stained for examination by transmission electron microscopy as described (Trier, J. S., et al., Gastroenterology 92:13-22 (1987)).

In pharmacologic experiments, detailed dose- and time-response studies are performed with the RORγt modulator which will also be administered by various routes (e.g., i.c., per-os (p.o.)). The colonic lesions are quantitated by computerized planimetry coupled with stereomicroscropy (Szabo, S., et al., J. Pharm. Methods 13:59-66 (1985)), and by a combination of damage score derived from gross and histologic examination of intestines, colonic weight and myeloperoxidase activity, as described by Morris et al. with the TNB model of IBD (Morris, G. P., et al., Gastroenterology 96:795-803 (1989)).

For biochemical studies, the tissue (total thickness, mucosa and muscle separated in certain experiments) is either homogenized with a Tekmar homogenizer, or kept frozen for up to two weeks.

For statistical evaluation, the results are stored and analyzed by computer. The statistical significance of differences of the group values are calculated (for parametric data) by two-tailed Student's t-test or (with parametric statistics) by the Mann-Whitney test or the Fisher-Yates Exact Probability Test.

### Example 3

### In Vivo Assessment of Modulators of RORγt in the Lysolecithin Model for Multiple Sclerosis

### Lysolecithin Induced Demyelination

For these experiments, 12 week old SJL/J mice are anesthetized with sodium pentobarbitol and a dorsal laminectomy is performed in the upper thoracic region of the spinal cord. A 34 guage needle attached to a Hamilton syringe is used to inject 1 ml of a 1% solution of lysolecithin directly into the dorsolateral aspect of the cord. Animals are killed on day 21 post injection and the injected region of the spinal cord is removed and processed for morphological evaluation.

As a second model of demyelination, intraspinal injection of lysolecithin is used. Twelve week old SJL/J mice are anesthetized by intraperitoneal injection of sodium pentobarbitol (0.08 mg/g). Dorsal laminectomies are performed on the upper thoracic region of the spinal cord and lysolecithin (L-lysophosphatidylcholine) (Sigma, St. Louis, Mo.) is injected as described (Pavelko, K. D., van Engelen, B. G. & Rodriguez, M. (1998) J. Neurosci. 18, 2498-2505). Briefly, a 34 gauge needle attached to a Hamilton syringe mounted on a stereotactic micromanipulator is used to inject 1% solution of lysolecithin in sterile PBS (pH 7.4) with Evan's blue added as a marker. The needle is inserted into the dorsolateral part of the spinal cord, 1 ul of lysolecithin solution is injected, and then the needle is slowly withdrawn. The wound is sutured in two layers, and mice are allowed to recover. The day of lysolecithin injection is designated day 0.

Seven days after lysolecithin injection, mice are treated with the RORγt modulator as a bolus intraperitoneal injection or intravenously. Initially a dose response study will be done to establish the most effective dose for use in this animal model. Control mice are treated with bolus intraperitoneal or intravenous injection of vehicle control. Three weeks and five weeks after the lysolecithin injection, mice are sacrificed and one mm thick sections are prepared. The araldite block showing the largest lysolecithin induced demyelination lesion is used for quantitative analysis. The total area of the lesion is quantitated using a Zeiss interactive digital analysis system. The total number of remyelinated fibers are quantitated using a Nikon microscope/computer analysis system. The data is expressed as the number of remyelinated axons/mm² of lesion.

Lysolecithin treated mice are given various doses of the RORγt modulator on days 0, 3, 7, 10, 14, and 17 after lysolecithin injection. Animals are killed on day 21 after lysolecithin injection. PBS or vehicle controls serve as negative controls.

### EAE Model

Experimental allergic encephalomyelitis (EAE) is a T cell mediated autoimmune disease of the central nervous system (CNS). Disease can be induced in susceptible strains of mice by immunization with CNS myelin antigens or alternatively, disease can be passively transferred to susceptible mice using antigen stimulated CD4+ T cells [Pettinelli, J. Immunol 127, 1981, p. 1420]. EAE is widely recognized as an acceptable animal model for multiple sclerosis in primates [Alvord et al. (eds.) 1984. Experimental allergic encephalomyelitis—A useful model for multiple sclerosis. Alan R. Liss, New York]. The effects of administration of an RORγt modulator, preferably an antagonist, on induction of EAE following the adoptive transfer of lymphocytes from immunized mice restimulated in vitro with a synthetic peptide of myelin proteolipid protein (PLP) will be studied. The experimental protocol for this model and preliminary results that demonstrate the role of RORγt in development and progression of disease in this model are shown in Example 6.

### Example 4

### In Vivo Assessment of Modulators of RORγt in a Model of Arthritis Arthritis

Inhibitory Effect of a RORγt Antagonist on Edema of Arthritis

In order to observe the inhibitory effect on edema of a pharmaceutical composition of the present invention, preferably one comprising a RORγt antagonist, 6 albino rats weighing 200 gm are used per test group and edema is induced by injecting a mixture of 0.5 ml of Zymosan-A (20 mg/ml/kg) and 0.5 ml of Freund's adjuvant into the left paw of the animals and the animals are observed for the progress of edema for 70 days by taking a photograph before and after induction of edema and by measuring the paw size with a caliper. Certain groups will be given various doses of the RORγt modulator (antagonist) after injection of the Zymosan-A and Freund's adjuvant. Administration may be via the intravenous route, the oral route, the intraperitoneal route or the subcutaneous route of injection. The water extract and organic solvent fractions of the pharmaceutical composition of the present invention (vehicle control) are respectively constituted in a concentration of 0.6 mg/ml and then administered for 14 days to albino rats in an amount of 1 ml per kg of body weight once a day to determine the inhibitory effect on edema. Edema is measured daily using a precision gauge, and photographs taken.

Similar studies may be done in the collagen model of arthritis (Myers, L. K. (1997), Life Sci. 61(19): 1861-1878).

### Example 5

### Animals Models for Studying the Effects of Modulators of RORγt on Proliferative (Cancerous) Disorders

### Cancer Vaccine Model

Studies will be done to determine whether the RORγt modulator can effectuate increased immunity to tumor antigens. For example, studies will be done to measure the in vivo growth of tumors, for example the Hepa 1-6 tumor cells or SMCC-1 colon carcinoma cells and the mortality associated with injection of these tumors to mice, when administered alone or in combination with a RORγt modulator.

To establish that immunization with tumor cells, for example, CT-hepa 1-6 cells or SMCC-1 colon carcinoma cells, when administered with a RORγt modulator can either cure established hepatomas or colon carcinoma, or prevent animals from developing tumors due to induction of an immune response, the following studies are performed. Any established animal/tumor model may be used.

In a first study, forty mice are divided into groups and all are inoculated subcutaneously with live 2×10⁶ hepa 1-6 cells or SMCC-1 cells. Some groups are treated with the tumor cells plus vehicle control and some are given various doses of the RORγt modulator at the time of injection of the tumor cells, (the RORγt modulator may be given either orally, IP, IM, IV or SC). The mice are monitored weekly for development of tumors. Mortality due to a large tumor burden is also monitored.

In another study, gamma-irradiated hepa 1-6 tumor cells or SMCC-1 cells are used as the vaccine. Three groups of ten mice per group are inoculated subcutaneously with gamma-irradiated 1×10⁶ hepa 1-6 cells or SMCC-1 cells. One group is treated with a vehicle control (PBS) at the time of injection of the irradiated tumor cells, the other two groups are given the RORγt modulator at two different doses (low and high) at the time of injection of the irradiated tumor cells. After two weeks, mice are then injected subcutaneously with 1×10⁶ live hepa 1-6 cells. The mice are then monitored weekly for tumor growth and mortality.

To further investigate if the increase in survival or the decrease in growth of tumors is due to induced immunity which may be mediated by CTLs, mice are depleted of CD8+T cells by antibody treatment before or after immunization. Depletion of CD8+ T cells either before or after immunization should abrogate the ability of the cellular vaccine to elicit anti-tumor immunity in vivo.

In addition, the animals injected with the tumor cells alone or in conjunction with the RORγt modulator may be sacrificed, the spleens removed and measurement of tumor specific cytolytic T cell activity measured in a standard 51Cr release assay, known to those skilled in the art. Antibodies made to the tumor antigen may also be monitored by testing the serum from the animals in standard ELISA assays.

### Example 6

### RORγt Directs the Differentiation Program of Pro-Inflammatory IL-17⁺ T Helper Cells

### Experimental Procedures

### Mice

Mice with a GFP reporter cDNA knocked-in at the site for initiation of RORγt translation (Eberl, G., Marmon, S., Sunshine, M. J., Rennert, P. D., Choi, Y., and Littman, D. R. (2004). An essential function for the nuclear receptor RORgamma(t) in the generation of fetal lymphoid tissue inducer cells. Nat Immunol 5, 64-73), as well as Rorγ^{-/-} mice on the C57BL/6 background (Sun, Z., Unutmaz, D., Zou, Y. R., Sunshine, M. J., Pierani, A., Brenner-Morton, S., Mebius, R. E., and Littman, D. R. (2000). Requirement for RORgamma in thymocyte survival and lymphoid organ development. Science 288, 2369-2373) have been described and were kept in SPF conditions at the animal facility of the Skirball Institute. Myd88^{-/-} mice were provided by Dr. Ruslan Medzhitov (Yale University). Ile⁻ mice on the C57BL/6 background were purchased from the Jackson laboratory and were provided by Dr. Joel Ernst (NYU). C57BL/6, Balb/c, and Rag2^{-/-} mice were purchased from Taconic. All animal experiments were performed in accordance with approved protocols from the NYU Institutional Animal Care and Usage Committee.

### EAE Induction and Disease Scoring

For induction of EAE, mice were immunized subcutaneously on day 0 with 150 mg/mouse MOG 35-55 peptide (Molecular Biology Core Facilities, Dana-Farber Cancer Institute, Harvard University) emulsified in CFA (CFA supplemented with 400 μg/ml Mycobacterium tuberculosis) and injected intravenously on days 0 and 2 with 200 ng/mouse of pertussis toxin (Calbiochem). The basic scoring system used was 0—no disease, 1—limp tail, 2—weak/partially paralyzed hind legs, 3—completely paralyzed hind legs, 4—complete hind and partial front leg paralysis, 5—complete paralysis/death. Mice with disease levels 4 and 5 were considered moribund and were euthanized.

### Bone Marrow Reconstitutions

Total bone marrow mononuclear cells were isolated from wild-type and Rorγ^{-/-} mice by flushing the long bones. Red blood cells were lysed with ACK Lysing Buffer (BioWhittaker) and the remaining mononuclear cells were resuspended in PBS for injection. 5-10×10⁶ cells per mouse were injected intravenously into 3-5 week-old Rag2^{-/-} mice that were sublethally irradiated using 400 rads/mouse 4 hours before reconstitution. EAE was induced 11 weeks post bone marrow reconstitution.

### CD4⁺ T Cell Transfers

Single cell suspensions were prepared from spleens of wildtype and Rorg^{-/-} mice and CD4⁺ cells purified by using anti-CD4 magnetic microbeads (Miltenyi Biotec) and MACS® columns (purity was >95%, usually 97-98%). 10⁷ CD4⁺ cells per mouse were injected intravenously into un-irradiated Rag2^{-/-} mice. EAE was induced 24 hours after transfer.

### Isolation of Lamina Propria Lymphocytes (LPLs) and Intraepithelial Lymphocytes (IELs)

Mice were killed and intestines removed and placed in ice cold PBS. After removal of residual mesenteric fat tissue, Peyer's patches were carefully excised, and the intestine was opened longitudinally. The intestine was then thoroughly washed in ice cold PBS and cut into 1.5 cm pieces. The pieces were incubated twice in 5 ml of 5 mM EDTA in HBSS for 15-20 min at 37° C. with slow rotation (100 rpm). After each incubation, the epithelial cell layer, containing the IELs, was removed by intensive vortexing and passing through a 100 μm cell strainer and new EDTA solution was added. After the second EDTA incubation the pieces were washed in HBSS, cut in 1 mm² pieces using razor blades, and placed in 5 ml digestion solution containing 4% fetal calf serum, 0.5 mg/ml each of Collagenase D (Roche) and DNase I (Sigma) and 50 U/ml Dispase (Fisher). Digestion was performed by incubating the pieces at 37° C. for 20 min with slow rotation. After the initial 20 min, the solution was vortexed intensely and passed through a 40 μm cell strainer. The pieces were collected and placed into fresh digestion solution and the procedure was repeated a total of three times. Supernatants from all three digestions (or from the EDTA treatment for IEL isolation) from a single small intestine were combined, washed once in cold FACS buffer, resuspended in 10 ml of the 40% fraction of a 40:80 Percoll gradient, and overlaid on 5 ml of the 80% fraction in a 15 ml Falcon tube. Percoll gradient separation was performed by centrifugation for 20 min at 2500 rpm at room temperature. LPLs were collected at the interphase of the Percoll gradient, washed once and resuspended in FACS buffer or T cell medium. The cells were used immediately for experiments.

Isolation of Mononuclear Cells from Spinal Cords

Before spinal cord (SC) dissection, mice were perfused with 25 ml 2 mM EDTA in PBS to remove blood from internal organs. The spinal columns were dissected, cut open and intact SCs separated carefully from the vertebrae. The SCs were cut into several small pieces and placed in 2 ml digestion solution containing 10 mg/ml Collagenase D (Roche) in PBS. Digestion was performed for 45 min at 37° C. with short vortexing every 15 min. At the end of the digestion the solution was vortexed intensively and passed through a 40 pm cell strainer. The cells were washed once in PBS, placed in 6 ml of 38% Percoll solution, and pelleted for 20 min at 2000 rpm. Pellets were resuspended in FACS buffer or T cell medium and used for subsequent experiments.

### Isolation of Total and Naïve CD4⁺ T Cells

CD4⁺ T cells were purified from spleens using anti-CD4 magnetic microbeads (Miltenyi Biotec) and MACS® columns (purity was >95%). For naive CD4⁺ T cells, single-cell suspensions were first negatively depleted by staining with anti-B220-PE, anti-CD8α-PE, anti-CD11b-PE, anti-CD11c-PE, anti-CD49b-PE, all at 1:100 dilution, and anti-Ter119-PE at 1:66 dilution, for 20 min on ice, followed by incubation with anti-PE magnetic microbeads (Miltenyi Biotec) at 1:20 dilution for 20 min on ice. The depleted fraction was stained with anti-CD25-PE, anti-CD4-PECy7, anti-CD62L-FITC and anti-CD44-APC. Cell sorting was performed on a MoFlo cytometer (DAKO Cytomation) to obtain a pure population of naïve CD4⁺CD25⁻ CD44^{low}CD62L⁺ T cells (>99% purity).

### Surface and Intracellular Cytokine Staining

For intracellular cytokine staining, cells obtained from in vitro culture or from dissection of lamina propria or spinal cords were incubated for 4-5 h with 50 ng/ml PMA (Sigma), 750 ng/ml Ionomycin (Sigma) and 10 μg/mlBrefeldin A (Invitrogen) in a tissue culture incubator at 37° C. Surface staining was performed for 15-20 min with the corresponding cocktail of fluorescently labeled antibodies. After surface staining, the cells were resuspended in Fixation/Permeabilization solution (BD Cytofix/Cytoperm kit—BD Pharmingen) and intracellular cytokine staining was performed as per the manufacturer's protocol.

### Flow Cytometry and Antibodies

Flow cytometric analysis was performed on LSR II (BD Biosciences) or FACSCalibur (BD Biosciences) instruments and analyzed using FlowJo software (Tree Star Inc.). All antibodies were purchased from BD Pharmingen or eBiosciences. In cases where intracellular cytokine staining was performed, GFP fluorescence was detected with anti-GFP-Alexa 488 (polyclonal rabbit IgG fraction—Molecular Probes).

### Plasmids and Retrovirus Production

The RORγt cDNA was PCR amplified and cloned into pMIG (RORγt-IRES-GFP). T-bet-IRES-GFP was a kind gift from Dr. Steve Reiner (University of Pennsylvania). Phoenix cells were transfected with 9 μg of the indicated plasmids using Lipofectamin 2000 (Invitrogen). Viral supernatant was collected and supplemented with 8 μg/ml polybrene (Sigma).

### Cell Culture and Retroviral Transduction

The T cell culture medium used was RPMI Media 1640 (Invitrogen) supplemented with 10% Fetal Calf Serum (Hyclone), 2 mM L-glutamine, 100 U/mL penicillin, 100 μg/mL streptomycin, 5 mM 2-β-mercaptoethanol. 1.5×10⁶/well MACS purified CD4⁺ T cells or sorted naive CD4⁺ T cells were cultured in 24-well plates (or 0.7×10⁶ cells per well in 48-well-plates) containing plate-bound anti-CD3 (5 μg/mL) and soluble anti-CD28 (1 μg/mL); cultures were supplemented with 40 U/mL mouse IL-2 (Roche), 10 μg/mL anti-IL-4 (BD Pharmingen), 10 μg/mL anti-IFN-γ (BD Pharmingen) with or without 20 ng/ml IL-6 (eBioscience) and 5 ng/ml TGF-β (Preprotech). For flow cytometry cells were analyzed on days 3 and 5.

For viral transduction, sorted naïve CD4⁺ T cells were plated as above in the absence of TGF-β and IL-6 on day 0. On day 1 and 2, fresh retrovirus supernatant was added and the cells were spun at 2500 rpm for 1.5 hours at 30° C. After spin infection, the cells were cultured in the T cell culture medium and harvested on day 5 or 6 for intracellular cytokine staining and real time PCR (RT-PCR) analysis.

### RT-PCR

cDNA was synthesized with RNA prepared by TRIZOL using RNase H-reverse transcriptase (Invitrogen). cDNA was analyzed by real-time quantitative PCR in triplicates by using iQ CYBR Green Supermix (Bio-Rad) or QuantiTect Multiplex PCR mix (Qiagen) in the iCycler Sequence Detection System (Bio-Rad). The starting quantity (SQ) of the initial cDNA sample was calculated from primer-specific standard curves by using the iCycler Data Analysis Software. The expression level of each gene was normalized to actin expression level using Standard Curve Method.

The primer sets for real-time PCR were as follows:

IL17:
(SEQ ID NO: 15)
5′-CTCCAGAAGGCCCTCAGACTAC-3′,
(SEQ ID NO: 16)
5′-AGCTTTCCCTCCGCATTGACACAG-3′,
IL 17 probe:
(SEQ ID NO: 17)
5′-FAM-TCTGGGAAGCTCAGTGCCGCCACCAGC-TAMRA-3′;
IL 17F:
(SEQ ID NO: 18)
5′-GAGGATAACACTGTGAGAGTTGAC-3′,
(SEQ ID NO: 19)
5′-GAGTTCATGGTGCTGTCTTCC-3′,
IL 17F probe:
(SEQ ID NO: 20)
5′-FAM-AGTTCCCCATGGGATTACAACATCACTC-TAMRA-37;
and
RORyt:
(SEQ ID NO: 21)
5′-CCGCTGAGAGGGCTTCAC-3′,
(SEQ ID NO: 22)
5′-TGCAGGAGTAGGCCACATTACA-3′,
RORyt probe:
(SEQ ID NO: 23)
5′-FAM-AAGGGCTTCTTCCGCCGCAGCCAGCAG-TAMRA-3′;
GAPDH
(SEQ ID NO: 24)
5′-TGGTGAAGGTCGGTGTGAAC-3′,
(SEQ ID NO: 25)
5′-CCATGTAGTTGAGGTCAATGAAGG-3′.

The primers and probes for IL-23R and actin were described elsewhere (Mangan, P. R., Harrington, L. E., O'Quinn, D. B., Helms, W. S., Bullard, D. C., Elson, C. O., Hatton, R. D., Wahl, S. M., Schoeb, T. R., and Weaver, C. T. (2006). Transforming growth factor-beta induces development of the T(H)17 lineage. Nature 441, 231-234; Egawa, T., Eberl, G., Taniuchi, I., Benlagha, K., Geissmann, F., Hennighausen, L., Bendelac, A., and Littman, D. R. (2005). Genetic evidence supporting selection of the Valphal4i NKT cell lineage from double-positive thymocyte precursors. Immunity 22, 705-716).

### Results

### RORγt is Expressed in a Subset of Lamina Propria T Cells and is Required for Expression of IL-17

To examine the role of RORγt in T cell development and lymphoid organogenesis we previously generated mice with a GFP reporter cDNA knocked-in at the site for initiation of RORγt translation (Eberl, G., Marmon, S., Sunshine, M. J., Rennert, P. D., Choi, Y., and Littman, D. R. (2004). An essential function for the nuclear receptor RORgamma(t) in the generation of fetal lymphoid tissue inducer cells. Nat Immunol 5, 64-73). Using these animals we found that RORγt is expressed at the double positive stage of T cell development but is absent in more mature thymocytes and in mature T cells in spleen and peripheral LNs (Eberl, G., Marmon, S., Sunshine, M. J., Rennert, P. D., Choi, Y., and Littman, D. R. (2004). An essential function for the nuclear receptor RORgamma(t) in the generation of fetal lymphoid tissue inducer cells. Nat Immunol 5, 64-73). In the periphery, we had found that GFP (or RORγt) expression was limited to a population of RORγt-dependent lymphoid-tissue inducer cells (LTi) in the fetus and a population with a similar phenotype in adult intestinal cryptopatches and lymphoid follicles (Eberl, G., Marmon, S., Sunshine, M. J., Rennert, P. D., Choi, Y., and Littman, D. R. (2004). An essential function for the nuclear receptor RORgamma(t) in the generation of fetal lymphoid tissue inducer cells. Nat Immunol 5, 64-73; Sun, Z., Unutmaz, D., Zou, Y. R., Sunshine, M. J., Pierani, A., Brenner-Morton, S., Mebius, R. E., and Littman, D. R. (2000). Requirement for RORgamma in thymocyte survival and lymphoid organ development. Science 288, 2369-2373). Upon closer examination of lamina propria cells from Rorγt^{gfp/+} mice, we observed subpopulations of TCRαβ⁺ and TCRγδ⁺ cells that expressed a lower level of GFP than the LTi-like TCR-negative cryptopatch cells. Approximately 10% of TCRαβ⁺ T cells, most of which were CD4⁺, and 50% of TCRγδ⁺ T cells expressed GFP (FIG. 11A). In Rorγt^{gfp/gfp} mice, which lack expression of RORγt, the LTi-like cells, which are Lin⁻GFP⁺, were completely absent. In contrast, the GFP⁺TCRαβ⁺ T cells were still present, although reduced by 50-75%, and the GFP⁺TCRγδ⁺ T cells were no longer observed (FIG. 11B). RORγt (GFP) expression was not detected in the intestinal epithelial cell (IEL) compartment of Rorγt^{gfp/+} or Rorγt^{gfp/gfp} mice (FIG. 18).

In experiments aimed at investigating the mechanisms that regulate Th17 cell differentiation, we performed Affymetrix gene chip analysis of CD4⁺ T cells cultured under conditions favoring Th17 (grown in IL-23) vs Th1 (grown in IL-12) differentiation. In Th17 culture conditions, RORγt mRNA levels were found to be elevated (B.S.M. and D.J.C., manuscript in preparation). The finding of RORγt⁺ T cells in the intestinal lamina propria therefore raised the possibility that these cells were of the Th17 lineage. When lamina propria lymphocytes (LPL) from Ror ⁺ mice were isolated and stimulated for 5 h with anti-CD3/CD28, we found that a large proportion of the GFP⁺ cells, but not the GFP⁻ cells, indeed expressed IL-17 (FIG. 12A).

In Rorγt^{gfp/+} and in wild-type mice, approximately 10% of CD4⁺TCRαβ⁺ intestinal T cells expressed IL-17 (FIG. 12B and data not shown). However, in RORγt-null (Rorγt^{gfp/gfp})mice, IL-17⁺ cells in the lamina propria were reduced by at least 10-fold to less than 1% of the CD4⁺ T cells, suggesting that RORγt may be necessary for the generation of Th17 cells in-vivo (FIG. 12B). Both IL-17 and RORγt (GFP) were also expressed in CD4⁻ T cells in the lamina propria and were reduced in RORγt-deficient mice (FIG. 12B and FIG. 19).

In mice kept under specific pathogen free conditions, IL-17⁺ cells were present in the lamina propria of the small intestine (proximal and distal), cecum, colon, and rectum. However, the small intestinal lamina propria contained the largest proportion of IL-17⁺ T cells and the cecum contained the smallest (FIG. 20). Very few cells in the IEL compartment (0.1-0.2% of TCRαβ and TCRγδ cells) expressed IL-17 (data not shown). We did not observe RORγt/GFP⁺ or IL-17⁺ T cells outside of the intestine in other secondary lymphoid organs. These observations suggest that signals from the intestinal microflora may regulate the differentiation of the intestinal Th17 cells in the lamina propria. In an effort to investigate this possibility, we assessed the presence of IL-17⁺ cells in the lamina propria of MyD88-deficient mice. We discovered only a mild decrease in the percentage of these cells in the mutant animals, indicating that if intestinal Th17 cells differentiate in response to signals from the lumen, then other signaling pathways are likely involved (FIG. 21).

### RORγt is Required for the In Vitro Induction of IL-17 in T Helper Cells

It was recently reported that purified naïve CD4⁺ T cells are induced to differentiate in vitro into Th17 cells upon antigen receptor ligation in the presence of IL-6 and TGF-β (Veldhoen, M., Hocking, R. J., Atkins, C. J., Locksley, R. M., and Stockinger, B. (2006). TGFbeta in the context of an inflammatory cytokine milieu supports de novo differentiation of IL-17-producing T cells. Immunity 24, 179-189). To test if RORγt is required for this differentiation program, we purified CD4⁺CD25⁻CD62⁺CD44⁻ naïve splenic T cells from wild-type and RORγt-deficient mice and stimulated them in vitro with plate bound anti-CD3 and soluble anti-CD28 for 3 days under various polarizing conditions. We confirmed that a combination of IL-6 and TGF-β in the presence of neutralizing antibodies against IFNγ and IL-4 resulted in robust induction of IL-17 in wild-type cells, and that either cytokine alone or IL-23 had no effect (FIG. 13A and data not shown). In contrast, CD4⁺ T cells from Rorγ^{-/-} mice (which lack both RORγand RORγt) displayed a marked reduction in IL-17⁺ cells after in vitro polarization. There was typically at least a 50-fold decrease in the number of IL-17⁺ cells, and the level of IL-17 staining in these cells was significantly reduced (FIG. 13A). Consistent with this observation, incubation of anti-CD3/CD28-stimulated T cells with IL-6 plus TGF-β resulted in induction of RORγt mRNA, as well as transcripts for IL-17 and IL-17F (FIG. 13B). The mRNA for RORγt peaked at 16 hours, whereas those for IL-17 and IL-17F peaked later, at 48 hours, consistent with RORγt regulation of IL-17 transcription. IL-6 and TGFβ on their own induced low levels of RORγt expression, but were unable to induce IL-17 mRNA or IL-17-producing T cells (FIG. 13C and data not shown).

The defect in RORγt-deficient mice was confined to IL-17 producing T cells, as differentiation of IFNγ-producing Th1 cells upon incubation with IL-12 was normal or even enhanced with cells from the mutant mice as compared to those from wild-type animals (FIG. 22).

### Forced Expression of RORγt Induces IL-17 in Naïve CD4⁺ T Cells

As RORγt appeared to be essential for Th17 cell differentiation in response to cytokines, we wished to determine whether its expression is sufficient to induce the Th17 lineage program in naïve T cells. We used retroviral transduction to deliver RORγt to magnetic bead (MACS)-purified CD4⁺ T cells or FACS-sorted CD4⁺CD25⁻CD62L⁺CD44⁻ T cells stimulated with anti-CD3/CD28. In the absence of exogenous polarizing cytokines, over-expression of RORγt resulted in the induction of IL-17 in a large fraction of the transduced (GFP⁺) T cells from C57BL/6 or Balb/c mice (FIG. 14A). In contrast, no IL-17 was observed in cells transduced with T-bet-IRES-GFP and IRES-GFP control vectors. Analysis by quantitative RT-PCR indicated that the forced expression of RORγt resulted in induced transcription of both IL-17 and IL-17F (FIG. 14B).

Transduction of the T-bet-encoding retrovirus resulted, as expected, in the induction of IFNγ, but not IL-17. Conversely, there was no IFNγ induction upon over-expression of RORγt. Instead, the number of IFNγ-producing cells was reduced in cells expressing RORγt (FIG. 14A). Even when the cells were cultured under Th1 polarizing conditions, RORγt expression still induced IL-17 expression (data not shown).

To eliminate any possibility that cytokines produced by contaminating antigen presenting cells contribute to the induction of IL-17 by RORγt, we repeated the experiments using highly purified naïve CD4⁺ T cells. Transduction of RORγt resulted in IL-17 expression in more than half of the cells in the absence of exogenous cytokines (FIG. 14C). Together, these results indicate that RORγt is sufficient to induce expression of IL-17 and IL-17F in antigen-stimulated naïve CD4⁺ T cells, and are consistent with a role for RORγt downstream of its induction by IL-6 and TGF-β.

### IL-6 is Required for RORγt-Expression in the Lamina Propria

IL-6-deficient mice have been shown to have normal numbers of T cells, but are defective in the induction of autoimmune diseases and display increased susceptibility to a variety of pathogens (Eugster, H. P., Frei, K., Kopf, M., Lassmann, H., and Fontana, A. (1998). IL-6-deficient mice resist myelin oligodendrocyte glycoprotein-induced autoimmune encephalomyelitis. Eur J Immunol 28, 2178-2187; Kopf, M., Baumann, H., Freer, G., Freudenberg, M., Lamers, M., Kishimoto, T., Zinkernagel, R., Bluethmann, H., and Kohler, G. (1994). Impaired immune and acute-phase responses in interleukin-6-deficient mice. Nature 368, 339-342). To determine if IL-6 is required in vivo for the differentiation of Th17 cells in the lamina propria, we characterized intestinal T cells from IL-6 mutant animals. Although lamina propria T cells were present in normal numbers in IL-6-deficient mice, IL-17⁺ cells were reduced by about 10-fold, similar to what was observed with RORγt-deficient mice (FIG. 15A).

Consistent with the flow cytometry data, quantitative RT-PCR performed with RNA from sorted lamina propria TCRβ⁺CD4⁺ cells demonstrated a reduction in IL-17 expression in the absence of IL-6. Moreover, in contrast to lamina propria CD4⁺ T cells from WT mice, those from IL-6-deficient mice did not express RORγt, IL-17F, and the IL-23-specific chain of the IL-23R (FIG. 15B). These data suggest that IL-6 upregulates RORγt and IL-23R in vivo in the lamina propria, thus promoting the generation of Th17 cells in the intestine.

### RORγt is Required for Th17-Mediated Autoimmune Inflammatory Disease

The experiments described above demonstrate that RORγt is a transcription factor required for differentiation of IL-17 producing Th17 cells in vitro and in the intestinal lamina propria. Since Th17 cells have been shown recently to be the major pathogenic population in several models of autoimmune inflammation, including EAE (Langrish, C. L., Chen, Y., Blumenschein, W. M., Mattson, J., Basham, B., Sedgwick, J. D., McClanahan, T., Kastelein, R. A., and Cua, D. J. (2005). IL-23 drives a pathogenic T cell population that induces autoimmune inflammation. J Exp Med 201, 233-240; Park, H., Li, Z., Yang, X. O., Chang, S. H., Nurieva, R., Wang, Y. H., Wang, Y., Hood, L., Zhu, Z., Tian, Q., and Dong, C. (2005). A distinct lineage of CD4 T cells regulates tissue inflammation by producing interleukin 17. Nat Immunol 6, 1133-1141), we investigated the role of RORγt in this model. EAE was induced in wild-type and RORγ-deficient mice by injecting MOG 35-55 peptide in complete Freund's adjuvant and pertussis toxin (PTX) at day 0 and PTX at day 2. Wild-type C57BL/6 mice developed disease on day 10-11 and by day 14 reached peak disease manifestation, with 80% reaching a score of 4. RORγ-deficient mice on the C57BL/6 background did not show signs of disease until day 22, at which point some mice developed mild disease (maximum score of 2) that quickly subsided (FIG. 16A). Thus, in the absence of endogenous RORγt mice are less susceptible to EAE.

Although RORγ-deficient mice contain largely normal splenic architecture, they lack all peripheral lymph nodes. It is therefore possible that the lack of lymph nodes alone may have contributed to the delayed and reduced autoimmunity observed in Rorγ^{-/-} animals. To address this caveat, we employed models in which EAE was induced after reconstitution of RAG2-deficient mice (Rag2^{-/-}), which contain all secondary lymphoid organs, including lymph nodes, with either CD4⁺ splenocytes or bone marrow from wild-type or Rorγ^{-/-} mice. Transfer of 1×10⁷ CD4⁺ splenocytes from wild-type mice, followed by induction of EAE 24 hours later, led to development of disease in all animals by day 15 post-induction. In contrast, transfer of CD4⁺ splenocytes from Rorγ^{-/-} mice resulted in only mild disease or, more commonly, in no disease (FIG. 16B).

In the bone marrow transfer model, we confirmed that the reconstitution efficiency was similar after 11 weeks in mice that received wild-type or RORγt-deficient cells (FIG. 23 and data not shown), and EAE was then induced. RAG-deficient mice reconstituted with wild-type bone marrow cells developed disease as early as day 10 after immunization (FIG. 16C). By day 14, 90% of the mice had severe disease (score of 4) and most animals were moribund and had to be sacrificed by day 20. In contrast, less than half of the RAG-deficient animals reconstituted with Rorγ^{-/-} bone marrow cells developed some disease, and this was relatively mild (FIG. 16C). Together, these results show that RORγt is required for fulminant EAE.

Despite the markedly reduced disease manifestations in mice that received bone marrow cells from Rorγ^{-/-} animals, infiltrating T cells were present in the spinal cords at levels similar to those in mice reconstituted with wild-type cells (FIG. 23). The total number of mononuclear infiltrating cells was slightly lower in the RORγ-deficient cell transfers, but did not reach statistical significance (3.4±1.3×10⁶ cells for WT, versus 2.0±1.1×10⁶ cells for RORγ-deficient mice on day 21). Mice that received wild-type bone marrow before induction of EAE contained both IFNγ and IL-17 producing T cells in the spinal cord infiltrate (FIGS. 16D and 16E). Remarkably, more than half of the IL-17-producing cells also expressed IFNγ. In contrast, although IFNγ-producing T cells were present in the RORγ-deficient T cell infiltrate, few of the infiltrating T cells produced IL-17 (FIGS. 16D and 16E), irrespective of the presence or absence of disease (FIG. 16D). In addition, MOG-specific IL-17⁺ cells could not be found in draining lymph nodes from mice reconstituted with bone marrow from mutant mice after 4 days of culture with MOG peptide, although similar numbers of total MOG-specific T cells were found in these cultures and those from control mice, suggesting a similar frequency of MOG-specific T cells in both groups (FIG. 24). Similar results were obtained in the analysis of mice in which EAE was induced after transfer of CD4⁺ splenocytes. Although infiltrating CD4⁺IFNγ⁺ T cells were present in both groups, only mice that received wild-type CD4⁺ T cells contained IL-17⁺ cells in the spinal cord and developed disease (FIG. 25).

During EAE, lymphoid and myeloid cells in the inflammatory infiltrate elicit increased expression of a number of proinflammatory cytokines and chemokines locally in the CNS. To further assess the etiology of the residual disease in the RORγ-deficient chimeras, we investigated the local CNS inflammatory responses by real time PCR. Induction of EAE in wild-type bone marrow chimeras led to increased expression of the pro-inflammatory cytokine genes previously reported, including IL-17, IL-17F, and IL-6, as well as the chemokines CCL6, CCL9, CCL11, CCL20, and CCL24, and the receptor CCR1 (data not shown). Consistent with the reduced number of Th17 cells, RORγ-deficient mice displayed significantly decreased levels of the Th17 cytokines and chemokines in the spinal cord during disease. However, IFNγ and IFNγ-regulated chemokines (including MIG) were unchanged in the RORγ-deficient chimeras, consistent with a primary role of pathogenic Th17, rather than Th1, cells in the disease process.

### Discussion

The experiments presented here have shown that the orphan nuclear receptor RORγt is the transcription factor that directs the differentiation of IL-17-producing inflammatory T cells. Recent studies have demonstrated that IL-17 is expressed at elevated levels in a variety of allergic and autoimmune diseases in humans (Barczyk, A., Pierzchala, W., and Sozanska, E. (2003). Interleukin-17 in sputum correlates with airway hyperresponsiveness to methacholine. Respir Med 97, 726-733; Fujino, S., Andoh, A., Bamba, S., Ogawa, A., Hata, K., Araki, Y., Bamba, T., and Fujiyama, Y. (2003). Increased expression of interleukin 17 in inflammatory bowel disease. Gut 52, 65-70; Infante-Duarte, C., Horton, H. F., Byrne, M. C., and Kamradt, T. (2000). Microbial lipopeptides induce the production of IL-17 in Th cells J Immunol 165, 6107-6115; Lock, C., Hermans, G., Pedotti, R., Brendolan, A., Schadt, E., Gan⁻en, H., Langer-Gould, A., Strober, S., Cannella, B., Allard, J., et al. (2002). Gene-microarray analysis of multiple sclerosis lesions yields new targets validated in autoimmune encephalomyelitis. Nat Med 8, 500-508; Witowski, J., Ksiazek, K., and Jon⁻es, A. (2004). Interleukin-17: a mediator of inflammatory responses. Cell Mol Life Sci 61, 567-579), and that Th17 cells represent a distinct lineage of T helper cells whose function is required in numerous animal models of autoimmune disease (Dong, C. (2006). Diversification of T-helper-cell lineages: finding the family root of IL-17-producing cells. Nat Rev Immunol 6, 329-333; McKenzie, B. S., Kastelein, R. A., and Cua, D. J. (2006). Understanding the IL-23-IL-17 immune pathway. Trends Immunol 27, 17-23). Within tissues, Th17 effector cells stimulate production of a variety of inflammatory chemokines, cytokines, metalloproteases, and other pro-inflammatory mediators and promote recruitment of granulocytes (Kolls, J. K., and Linden, A. (2004). Interleukin-17 family members and inflammation. Immunity 21, 467-476; Stamp, L. K., James, M. J., and Cleland, L. G. (2004). Interleukin-17: the missing link between T-cell accumulation and effector cell actions in rheumatoid arthritis? Immunol Cell Biol 82, 1-9). We found that RORγt is required for the constitutive expression of IL-17 in intestinal lamina propria T cells and for the in vitro differentiation of Th17 cells from naïve CD4⁺ T cells. Following ligation of the antigen receptor, the cytokines IL-6 and TGF-β together induce the transcription of RORγt, which, in turn, participates in and may be sufficient for the induction of IL-17 expression. We have additionally shown a central role for RORγt in the in vivo differentiation of pathogenic Th17 cells within the central nervous system in a model for inflammatory autoimmune disease, EAE. Together, these studies suggest that manipulation of RORγt activity may be an attractive therapeutic strategy in a variety of diseases with inflammatory etiology.

### Regulation of the Th17 Differentiation Pathway by RORγt

RORγ and RORγt are both encoded within the Rorc locus, and differ only in their amino terminal sequences due to utilization of different promoters (Eberl, G., and Littman, D. R. (2003). The role of the nuclear hormone receptor RORgammat in the development of lymph nodes and Peyer's patches. Immunol Rev 195, 81-90). Both are members of the retinoic acid related orphan nuclear hormone receptor family that also includes RORα and RORβ (Jetten, A. M. (2004). Recent advances in the mechanisms of action and physiological functions of the retinoid-related orphan receptors (RORs). Cuff Drug Targets Inflamm Allergy 3, 395-412). Whereas RORγ is expressed broadly, and as yet has no defined function, RORγt is expressed exclusively in cells of the immune system.

We previously showed that RORγt, but not RORγ, is expressed in fetal LTi cells, intestinal LTi-like cells, and in immature thymocytes (Eberl, G., and Littman, D. R. (2004). Thymic origin of intestinal alphabeta T cells revealed by fate mapping of RORgammat+ cells. Science 305, 248-251; Sun, Z., Unutmaz, D., Zou, Y. R., Sunshine, M. J., Pierani, A., Brenner-Morton, S., Mebius, R. E., and Littman, D. R. (2000). Requirement for RORgamma in thymocyte survival and lymphoid organ development. Science 288, 2369-2373). In this study, we found that sub-populations of intestinal lamina propria T cells also express low levels of RORγt, and many of these cells constitutively express IL-17. In vitro, IL-6 and TGF-β induced transcription of RORγt in purified CD4⁺ T cells prior to the onset of IL-17 and IL-17F expression. The expression of IL-17 in the intestinal T cells and in cytokine-stimulated CD4⁺ T cells was dependent on the presence of RORγt. In addition, lamina propria T cells from IL-6-deficient mice did not express RORγt or IL-17. Thus it is likely that, after T cells migrate into the lamina propria, RORγt is induced locally by the combination of IL-6 and TGF-β, which are abundant at this site, resulting in differentiation of Th17 cells (FIG. 17).

It was recently shown that TGF-β induces expression of Foxp3 and promotes the differentiation of regulatory T cells (Tregs) (Chen, W., Jin, W., Hardegen, N., Lei, K. J., Li, L., Marinos, N., McGrady, G., and Wahl, S. M. (2003). Conversion of peripheral CD4+CD25− naive T cells to CD4+CD25+ regulatory T cells by TGF-beta induction of transcription factor Foxp3. J Exp Med 198, 1875-1886; Fantini, M. C., Becker, C., Monteleone, G., Pallone, F., Galle, P. R., and Neurath, M. F. (2004). Cutting edge: TGF-beta induces a regulatory phenotype in CD4+CD25− T cells through Foxp3 induction and down-regulation of Smad7 J Immunol 172, 5149-5153). This program was blocked by IL-6, which together with TGF-β induced IL-17 expression instead (Bettelli, E., Carrier, Y., Gao, W., Korn, T., Strom, T. B., Oukka, M., Weiner, H. L., and Kuchroo, V. K. (2006). Reciprocal developmental pathways for the generation of pathogenic effector TH17 and regulatory T cells. Nature 441, 235-238). The relative balance of IL-6 and TGF-β may therefore control the local differentiation of Tregs and Th17 cells at steady state in the intestine and in inflammatory conditions in diverse tissues (FIG. 17). Paradoxically, TGF-β and IL-6 can individually induce expression of some RORγt, but neither alone can induce IL-17. This may in part be explained by the ability of Foxp3 to inhibit RORγt-induced IL-17 expression (L.Z. & D.R.L., unpublished). It will be important to further dissect the transcriptional networks that govern differential expression of Foxp3 and RORγt in response to the cytokines. IFNγ and IL-4 have also been reported to interfere with Th17 differentiation in vitro, raising the possibility that there is reciprocal inhibition between the different T helper cell differentiation pathways.

IL-23 is also involved in Th17 cell differentiation, but naïve T cells are IL-23 receptor negative and relatively refractory to IL-23 stimulation (Langrish, C. L., Chen, Y., Blumenschein, W. M., Mattson, J., Basham, B., Sedgwick, J. D., McClanahan, T., Kastelein, R. A., and Cua, D. J. (2005). IL-23 drives a pathogenic T cell population that induces autoimmune inflammation. J Exp Med 201, 233-240; Oppmann, B., Lesley, R., Blom, B., Timans, J. C., Xu, Y., Hunte, B., Vega, F., Yu, N., Wang, J., Singh, K., et al. (2000). Novel p19 protein engages IL-12p40 to form a cytokine, IL-23, with biological activities similar as well as distinct from IL-12 Immunity 13, 715-725). Only following in vivo priming does in vitro stimulation of T cells with antigen and antigen presenting cells plus IL-23 result in effective expansion of Th17 cells (Langrish, C. L., Chen, Y., Blumenschein, W. M., Mattson, J., Basham, B., Sedgwick, J. D., McClanahan, T., Kastelein, R. A., and Cua, D. J. (2005). IL-23 drives a pathogenic T cell population that induces autoimmune inflammation. J Exp Med 201, 233-240; Murphy, C. A., Langrish, C. L., Chen, Y., Blumenschein, W., McClanahan, T., Kastelein, R. A., Sedgwick, J. D., and Cua, D. J. (2003). Divergent pro- and antiinflammatory roles for IL-23 and IL-12 in joint autoimmune inflammation. J Exp Med 198, 1951-1957; Veldhoen, M., Hocking, R. J., Atkins, C. J., Locksley, R. M., and Stockinger, B. (2006). TGFbeta in the context of an inflammatory cytokine milieu supports de novo differentiation of IL-17-producing T cells. Immunity 24, 179-189). Our results suggest that IL-23R is upregulated on RORγt⁺ Th17 cells in an IL-6-dependent manner (FIG. 15; L.Z. and B.S.M., unpublished results). IL-23 may therefore function subsequent to IL-6/TGF-β-induced commitment to the Th17 lineage to promote cell survival and expansion, and, potentially, continued expression of IL-17 and other cytokines that characterize the Th17 phenotype (FIG. 17). It is not yet known if RORγt is required for sustained IL-17 expression in response to IL-23R-mediated signals in effector/memory T cells.

### Role of RORγt in Immune System Homeostasis

In healthy adult mice, RORγt has been detected only in immature thymocytes and in cells residing in the intestinal lamina propria. In the intestine, RORγt is required for the differentiation of Th17 cells and of LTi-like cells that form cryptopatches and appear to be critical for differentiation of isolated lymphoid follicles (ILFs) (Eberl, G., and Littman, D. R. (2004). Thymic origin of intestinal alphabeta T cells revealed by fate mapping of RORgammat+ cells. Science 305, 248-251). It is intriguing that two types of RORγt-dependent cells with very different mechanisms of action are both positioned at a mucosal surface that is in constant interaction with an enormous amount of microbial flora. The cryptopatch cells and the Th17 cells may be regulated coordinately, perhaps by a ligand for RORγt, to maintain homeostasis of the microflora and integrity of the epithelial barrier. Cryptopatches are located at the base of intestinal crypts and are thus positioned to receive signals from the intestinal lumen and transmit them to other cells in the lamina propria and to crypt epithelial cells. Development of mature ILFs requires signals from the gut flora (Lorenz, R. G., Chaplin, D. D., McDonald, K. G., McDonough, J. S., and Newberry, R. D. (2003). Isolated lymphoid follicle formation is inducible and dependent upon lymphotoxin-sufficient B lymphocytes, lymphotoxin beta receptor, and TNF receptor I function. J Immunol 170, 5475-5482; Pabst, O., Herbrand, H., Worbs, T., Friedrichsen, M., Yan, S., Hoffmann, M. W., Korner, H., Bernhardt, G., Pabst, R., and Forster, R. (2005). Cryptopatches and isolated lymphoid follicles: dynamic lymphoid tissues dispensable for the generation of intraepithelial lymphocytes. Eur J Immunol 35, 98-107; Taylor, R. T., and Williams, I. R. (2005). Lymphoid organogenesis in the intestine Immunol Res 33, 167-181) that may induce recruitment of B lymphocytes to cryptopatches (Ivanov, I. I., Diehl, G. E., and Littman, D. R. (2006). Lymphoid Tissue Inducer Cells in Intestinal Immunity CTMI 308, 59-82). Signals from the gut flora may also be responsible for the unique presence of Th17 cells in the intestinal mucosa.

Th17 cells are likely to be required to control infections at mucosal surfaces. IL-23-deficient mice are unable to control orally administered Citrobacter rodentium, which stimulates expansion of lamina propria Th17 cells (Mangan, P. R., Harrington, L. E., O'Quinn, D. B., Helms, W. S., Bullard, D. C., Elson, C. O., Hatton, R. D., Wahl, S. M., Schoeb, T. R., and Weaver, C. T. (2006). Transforming growth factor-beta induces development of the T(H)17 lineage. Nature 441, 231-234). IL-17 and IL-23 have also been shown to be important in protecting mice from lung infection with Klebsiella pneumoniae (Happel, K. I., Dubin, P. J., Zheng, M., Ghilardi, N., Lockhart, C., Quinton, L. J., Odden, A. R., Shellito, J. E., Bagby, G. J., Nelson, S., and Kolls, J. K. (2005). Divergent roles of IL-23 and IL-12 in host defense against Klebsiella pneumoniae. J Exp Med, jem.20050193; Ye, P., Rodriguez, F. H., Kanaly, S., Stocking, K. L., Schurr, J., Schwarzenberger, P., Oliver, P., Huang, W., Zhang, P., Zhang, J., et al. (2001). Requirement of interleukin 17 receptor signaling for lung CXC chemokine and granulocyte colony-stimulating factor expression, neutrophil recruitment, and host defense. J Exp Med 194, 519-527). By inducing chemokine and G-CSF expression from surrounding cells, RORγt-dependent Th17 cells promote expansion and recruitment of neutrophils that can control these microorganisms if they breach the epithelial barrier. In vitro studies using intestinal epithelial cell cultures showed that IL-17 could enhance tight junction formation that is crucial for the protective function of the gut mucosa (Kinugasa, T., Sakaguchi, T., Gu, X., and Reinecker, H. C. (2000). Claudins regulate the intestinal barrier in response to immune mediators. Gastroenterology 118, 1001-1011). In an acute model of colitis induced by dextran sulfate sodium, which strips the intestinal mucosal barrier, neutralization of IL-17 exacerbated colonic inflammation (Ogawa, A., Andoh, A., Araki, Y., Bamba, T., and Fujiyama, Y. (2004). Neutralization of interleukin-17 aggravates dextran sulfate sodium-induced colitis in mice. Clin Immunol 110, 55-62). These results suggest that RORγt-mediated induction of IL-17 expression can also have a protective or regenerative function when epithelial damage occurs.

It is unknown if RORγt directly induces IL-17 transcription in Th17 cells. ROR response elements (ROREs) consist of the consensus core motif AGGTCA preceded by a 5-bp A/T-rich sequence (Jetten, A.M., Kurebayashi, S., and Ueda, E. (2001). The ROR nuclear orphan receptor subfamily: critical regulators of multiple biological processes. Prog Nucleic Acid Res Mol Biol 69, 205-247). We performed a search for potential ROREs within the il17 locus and identified an evolutionarily conserved RORγt binding site within the 200 bp core promoter region, suggesting that transcription of IL-17 may be directly regulated by RORγt (data not shown). Whether RORγt activity is regulated by a ligand is not yet known. Exposure to a ligand in the intestine could potentially regulate the transcriptional activity of RORγt and may thus govern formation of follicles, differentiation of effector T cells, and even the dynamic equilibrium of the epithelial barrier.

### Central Role of RORγt in Inflammatory Diseases

During the past year, it has become increasingly clear that T helper cells that secrete IL-17, rather than IFNγ-producing Th1 cells, are the major inflammatory cells in a number of disease models, and that function of these cells is dependent on IL-23 (Chen, Y., Langrish, C. L., McKenzie, B., Joyce-Shaikh, B., Stumhofer, J. S., McClanahan, T., Blumenschein, W., Churakovsa, T., Low, J., Presta, L., et al. (2006). Anti-IL-23 therapy inhibits multiple inflammatory pathways and ameliorates autoimmune encephalomyelitis. J Clin Invest 116, 1317-1326; Cua, D. J., Sherlock, J., Chen, Y., Murphy, C. A., Joyce, B., Seymour, B., Lucian, L., To, W., Kwan, S., Churakova, T., et al. (2003). Interleukin-23 rather than interleukin-12 is the critical cytokine for autoimmune inflammation of the brain. Nature 421, 744-748; Langowski, J. L., Zhang, X., Wu, L., Mattson, J. D., Chen, T., Smith, K., Basham, B., McClanahan, T., Kastelein, R. A., and Oft, M. (2006). IL-23 promotes tumour incidence and growth. Nature May 10, e-pub; Mangan, P. R., Harrington, L. E., O'Quinn, D. B., Helms, W. S., Bullard, D. C., Elson, C. O., Hatton, R. D., Wahl, S. M., Schoeb, T. R., and Weaver, C. T. (2006). Transforming growth factor-beta induces development of the T(H)17 lineage. Nature 441, 231-234; Yen, D., Cheung, J., Scheerens, H., Poulet, F., McClanahan, T., McKenzie, B., Kleinschek, M. A., Owyang, A., Mattson, J., Blumenschein, W., et al. (2006). IL-23 is essential for T cell-mediated colitis and promotes inflammation via IL-17 and IL-6. J Clin Invest 116, 1310-1316). In EAE, Th17 cells are far more potent than IFNγ-producing Th1 cells, with as few as 1×10⁵ IL-17-producing T cells sufficient to transfer disease to naïve recipients (Langrish, C. L., Chen, Y., Blumenschein, W. M., Mattson, J., Basham, B., Sedgwick, J. D., McClanahan, T., Kastelein, R. A., and Cua, D. J. (2005). IL-23 drives a pathogenic T cell population that induces autoimmune inflammation. J Exp Med 201, 233-240). We found that RORγt is required for pathogenic Th17 responses in EAE. In the CNS of animals with RORγt-deficient T cells, the cytokines characteristic of Th17 cells were significantly reduced, but Th1 cytokines were normally expressed. The absence of Th17 cells led to a decrease in pro-inflammatory chemokines, consistent with the role of the IL-23-IL-17 pathway in recruiting inflammatory cells to sites of inflammation (McKenzie, B. S., Kastelein, R. A., and Cua, D. J. (2006). Understanding the IL-23-IL-17 immune pathway. Trends Immunol 27, 17-23; Stark, M. A., Huo, Y., Burcin, T. L., Morris, M. A., Olson, T. S., and Ley, K. (2005). Phagocytosis of apoptotic neutrophils regulates granulopoiesis via IL-23 and IL-17. Immunity 22, 285-294). RORγt-dependent mechanisms that normally regulate host defense by promoting neutrophil mobilization and tissue repair are therefore also critical in initiating tissue specific inflammatory responses.

Our results indicate that RORγt has a role in Th17 differentiation that resembles the roles of T-bet and GATA-3 in the differentiation of Th1 and Th2 cells, respectively. Unlike these other transcription factors, RORγt is a nuclear receptor with a ligand-binding pocket, and it is hence likely to be an excellent target for pharmacologic intervention in inflammatory diseases that result in autoimmunity and cancer progression.

### Example 7

### Site Directed Mutagenesis in RORγt: Effect on RORγt Function in Th17 Cells

Supporting evidence that a ligand binds to the “ligand binding domain” (LBD) of RORγt, and that interference or enhancement of such binding can modulate RORγt activity is provided by the following studies.

As described herein, RORγt is both necessary and sufficient for Th17 cell development. Unlike its family members (RORα and RORβ), no ligand for RORγt has yet been identified. However, based on structural and sequence analysis, RORγt is predicted to have an open ligand binding pocket (LBP).

A schematic drawing of the various domains within the RORγt molecule is shown in FIG. 26. DBD is the DNA Binding Domain; LK is the linker region; LBD is the Ligand Binding Domain and AF2 is the activation domain.

An experiment was done to determine the role or effect of each of the various domains in RORγt function as related to IL17 producing cells. The bottom panel shows the effect of replacement of the LBD and AF2 domains with a VP16 viral vector. As shown in the bottom panel, wild type RORγt (right side) was effective at enhancing the number of IL17 producing cells, as compared to the empty vector control (left side, MIG). A vector containing RORγt-VP16 was prepared. This construct replaced the ligand binding domain/AF2 domain with VP-16 to determine its effect on IL17 producing cells. The results demonstrated that the LBD and AF2 domain can be substituted with the VP16 activation domain without significant alteration in IL-17 expression/production. This result is consistent with LBD/AF2 recruitment of co-activator molecules, a function that can be replaced with VP16.

Using site directed mutagenesis, various other mutations were introduced into the various domains to help elucidate the role of each domain on the effect of RORγt on IL17 producing cells. For example, the RORγt-N1 mutant contained no DBD. The RORγt-N2 mutant contained no DBD and no LK region. The RORγt-C1 mutant contained no LBD or AF2 domain. The RORγt-C2 mutant contained no AF2 domain. The RORγt-VP16 mutant contained a VP-16 viral vector in place of the AF2 domain. The RORγt-304F/V contained either a valine in place of the alanine at position 304, or a phenylalanine in place of the alanine at position 304 of the RORγt gene (a position predicted to line the ligand binding pocket in RORγt).

The amino acids around the LBP are highly conserved between RORγt and RORβ (FIG. 27, left panel, the circled amino acids are conserved between RORγt and RORβ). A304 in RORγt (A269 in RORβ) is an amino acid lining the surface of the LBP. We reasoned that exchanging A304 with a bulky side-chain amino acid (i.e. Phe) may disrupt the binding pocket of RORγt, thereby preventing its potential ligand from binding and consequently interfering with RORγt's function. Using site-directed mutagenesis, we introduced two mutations to the amino acid 304 (A304F and A304V). Interestingly, the A304F mutant completely abolished RORγt 's function in Th17 cell production. Not surprisingly, the A304V mutant functions nearly as well as wildtype in both IL-17 production and CD8 down-regulation, since its side chain is not as bulky as Phe. (FIG. 27, right panel)

In FIG. 28, naïve CD4+ T cells were transduced with one of the following genetic constructs as described schematically in FIG. 26, and outlined below, and the number of IL17 producing cells was monitored by fluorescence activated cell sorting:

A: MIG: empty retrovirus vector control

B: wild type (wt) RORγt

C: RORγt A304V (change in A=alanine to V=valine)

D: RORγt A304F (change in A=alanine to F=phenylalanine)

E: RORγt N1

F: RORγt N2

G: RORγt C1

H: RORγt C2

As shown in the figure, the MIG control (A) showed no increase in IL17 producing cells, whereas the RORγt wt control (B) showed a significant increase in IL17 producing cells within the transduced GFP+ population. Transduction of the cells with the RORγt A304V mutant construct (C) showed no significant difference from the wt-RORγt containing cells with respect to the number of IL17 producing cells. Transduction of cells with the RORγt A304F construct (D), however, abolished the increase in IL17 producing cells observed with wt RORγt, suggesting that this site is crucial for the effect of RORγt on inducing IL17 producing cells. The cells transduced with all of the other constructs (E, F, G and H) showed a significant impairment in the induction of IL17 producing cells, as compared to WT RORγt, suggesting that all of the domains shown in FIG. 26 are important for retaining the total functionality of RORγt as related to IL17 cell production.

In FIG. 29, naïve CD4+ T cells were transduced with one of the following genetic constructs as described schematically in FIG. 26, and outlined below, and the number of interferon gamma (IFN-γ) producing cells was monitored by fluorescence activated cell sorting:

A: MIG: empty retrovirus vector control

B: wild type (wt) RORγt

C: RORγt A304V (change in A=alanine to V=valine at position 304 of RORγt in SEQ ID NO: 3)

D: RORγt A304F (change in A=alanine to F=phenylalanine at position 304 of RORγt in SEQ ID NO: 3)

E: RORγt N1

F: RORγt N2

G: RORγt C1

H: RORγt C2

As shown in the figure, the MIG empty vector control (A) showed the presence of IFN-γ producing cells, whereas the RORγt wt control (B) showed a significant decrease in IFN-γ producing cells. Transduction of the cells with the RORγt A304V mutant construct (C) showed no significant difference from the wt-RORγt containing cells with respect to the number of IFN-γ producing cells. Transduction of cells with the RORγt A304F construct (D), however, showed no difference in the number of IFN-γ producing cells compared to empty vector, suggesting that this site, which is crucial for the effect of RORγt on inducing IL17 producing cells, also has an effect on inhibition of expression of IFNγ. The cells transduced with all of the other constructs (E, F, G and H) also showed no reduction in the induction of IFNγ, suggesting that there is a relationship between the domains shown in FIG. 26, which are important for retaining the total functionality of RORγt as related to IL17 cell production, and the ability of RORγt to induce IFNγ.

FIG. 30 shows an immunoblot to study the expression levels of the wild type and mutant forms of RORγt. Cells were transduced with the MIG vector control, the wt RORγt, and with all of the mutants described in FIG. 26. Cells were lysed and run on electrophoretic gels, then transferred to nitrocellulose filters and blotted with a hamster monoclonal antibody specific for RORγt. alphaHMG-1 was used as a protein loading control in all lanes. As shown, all of the RORγt constructs are recognized by the hamster antibody, except for the N2 mutant, which lacks the epitope recognized by the mAb.

### Example 8

### Induction of Colitis by Transfer of CD45RBhi CD4+CD25− Cells into RAG KO Mice

A study was done to determine whether colitis could be induced by the transfer of CD4+ T cells from either RORγt-wild type animals or RORγt knockout animals into RAG2-deficient mice using the colitis model described by Powrie et al (Uhlig, H H et al. Immunity (2006), August; 25(2):309-318; Mottet, c. et al. J. Immunology (2003) Apr 15; 170(8):3939-3943; Uhlig, H H and Powrie, F. Springer Semin. Immunopathol. (2005) June; 27(2):167-180) This model has been shown to result in colitis if regulatory T cells are absent from the CD4+ naïve T cell population. After cell transfer , the animals were weighed daily to monitor body weight loss. As shown in the figure, T cells from RORγt KO animals showed no significant weight loss, thus suggesting the absence of an active disease process. However, animals receiving T cells from RORγt wild type animals showed significant weight loss over a time period of greater than 100 days, suggesting that the health status of these animals was impaired. The animals were sacrificed and their intestines and colons were removed and cells were isolated and analyzed for the presence of Th17 producing cells. Animals receiving CD4+ T cells from wild type RORγt animals showed a significantly higher number of Th17 producing cells in the small intestine and colon as compared to animals receiving CD4+ cells from KO animals, suggesting a role for RORγt in the production of IL17 producing cells in vivo.

### Example 9

### Synthesis of a shRNA Molecule that Inhibits Expression of wt RORγt

Two shRNA molecules were generated using the DNA sequence of SEQ ID NO: 3 (mouse RORγt). In particular, the nucleotides from position 358-378 of SEQ ID NO: 3 and from position 1269-1289 of SEQ ID NO: 3 (mouse RORγt) were used to generate shRNA molecules. These sequences are as follows: GGAGCAGACACACTTACATAC (SEQ ID NO: 9) and GGAACTGGCTTTCCATCATCA (SEQ ID NO: 10). When tested for the ability to inhibit expression of RORγt, as well as expression of a target molecule for RORgt, significant inhibition of expression was observed. (See FIGS. 42 and 43).

Table S3 below shows the description of relevant sequences described herein.

TABLE S3
Description of Sequence Listing

| | Nucleic Acid/Protein | GenBank Accession |
|---|---|---|
| SEQ ID NO: | And Description | No. (If appropriate) |
| 1 | human RORγt DNA | NM_001001523 |
| 2 | human RORγt protein | NP_001001523 |
| 3 | mouse RORγt DNA | AF_163668 |
| 4 | mouse RORγt protein | AAD 46913 |
| 5 | human RORγ DNA | NM_005060 |
| 6 | human RORγ protein | NP_005051 |
| 7 | mouse RORγ DNA | NM_011281 |
| 8 | mouse RORγ protein | NP_035411 |
| 9 | mouse RORγt DNA | Synthetic sequence |
| 10 | mouse RORγt DNA | Synthetic sequence |
| 11 | human RORγ DNA | U16997.1 |
| 12 | human RORγ protein | U16997.1 |
| 13 | mouse RORγ DNA | AF019657 |
| 14 | mouse RORγ protein | AF019657 |
| 15 | DNA | IL-17 primer |
| 16 | DNA | IL-17 primer |
| 17 | DNA | IL-17 probe |
| 18 | DNA | IL-17 F primer |
| 19 | DNA | IL-17 F primer |
| 20 | DNA | IL-17 F probe |
| 21 | DNA | RORγt primer |
| 22 | DNA | RORγt primer |
| 23 | DNA | RORγt probe |
| 24 | DNA | GAPDH primer |
| 25 | DNA | GAPDH primer |

1. A method for inhibiting the formation of immune cell aggregates, said aggregates comprising isolated lymphoid follicles, and colonic patches in the gut of a mammal, comprising administering an inhibitor or antagonist of RORγt, wherein the inhibitor or antagonist is a short double stranded RNA that binds to RORγt mRNA and inhibits expression of RORγt.

2. The method of claim 1, wherein cells of said immune cell aggregates that are inhibited are selected from the group consisting of cryptopatch (CP) cells and Th-IL17 cells.

3. (canceled)

4. (canceled)

5. The method of claim 2, wherein said method further results in a reduction in the number of αβT cells, wherein said αβT cells are selected from the group consisting of CD4⁻8⁻ T cells, CD4+ T cells, CD8αβ+ T cells, CD8αα+ T cells and Th-IL17 cells.

6. The method of claim 5, wherein said reduction in αβT cells occurs in the intestine.

7. A method of treating inflammatory and/or autoimmune diseases, comprising administering an inhibitor or antagonist of RORγt, wherein the inhibitor or antagonist is a short double stranded RNA that binds to RORγt mRNA and inhibits expression of RORγt.

8. The method of claim 7, wherein the treating results in a decrease in ectopic lymphoid follicle formation and/or a decrease in Th-IL17 cells.

9. The method of claim 7, wherein said diseases are selected from the group consisting of arthritis, diabetes, multiple sclerosis, uveitis, rheumatoid arthritis, psoriasis, asthma, bronchitis, allergic rhinitis, chronic obstructive pulmonary disease, atherosclerosis, H. pylori infections and ulcers resulting from such infection, and inflammatory bowel diseases.

10. The method of claim 9, wherein said inflammatory bowel diseases are selected from the group consisting of Crohn's disease, ulcerative colitis, sprue and food allergies.

11-25. (canceled)

26. A method of inhibiting the induction, expression and/or release of a pro-inflammatory cytokine or a pro-inflammatory cytokine receptor and/or a pro-inflammatory chemokine or a pro-inflammatory chemokine receptor, comprising administering an inhibitor or antagonist of RORγt, wherein the inhibitor or antagonist is a short double stranded RNA that binds to RORγt mRNA and inhibits expression of RORγt.

27. (canceled)

28. The method of claim 26, wherein the pro-inflammatory cytokine is selected from the group consisting of IL-17, IL-17F, IL-6, IL-21, IL-22, TNFsf8 and TNF-alpha.

29-31. (canceled)

32. The method of claim 26, wherein the administering is in vitro or in vivo.

33. The method of claim 32, wherein the in vivo administering results in a reduction in the severity of an inflammatory or autoimmune disease or condition, or an amelioration of one or more symptoms or sequelae associated with an inflammatory or autoimmune disease or condition.

34. The method of claim 33, wherein the inflammatory or autoimmune disease or condition is selected from the group consisting of arthritis, diabetes, multiple sclerosis, inflammation associated with an acute or chronic spinal cord injury, or inflammation associated with brain trauma, uveitis, rheumatoid arthritis, psoriasis, asthma, bronchitis, allergic rhinitis, chronic obstructive pulmonary disease, arteriosclerosis, H. pylori infections and ulcers resulting from such infection and an inflammatory bowel disease.

35. The method of claim 34, wherein the inflammatory bowel disease is selected from the group consisting of Crohn's disease, ulcerative colitis, sprue and a food allergy.

36-78. (canceled)

79. The method of claim 1, wherein the short double stranded RNA is a small interfering RNA (siRNA) or short hairpin RNA (shRNA).

80. The method of claim 1, wherein the short double stranded RNA is transcribed from a nucleic acid vector.

81. The method of claim 79, wherein the shRNA comprises the nucleic acid sequence of SEQ ID NO: 9 or SEQ ID NO: 10.
